(19) United States
(12) Patent Application Publication
(10) Pub. No.: US 2025/0270599 A1
(43) Pub. Date: Aug. 28, 2025

(54) METHOD OF MAKING GLYCOMONOLIPIDS

(71) Applicant: The Procter & Gamble Company, Cincinnati, OH (US)

(72) Inventors: Hilda Andamiche NAMANJA-MAGLIANO, Loveland, OH (US); Jose Carlos GARCIA-GARCIA, Cincinnati, OH (US); Tomislav TICAK, Mason, OH (US); Betzabe Rachel GALVEZ, Hamilton, OH (US); Thomas Shaw MOODY, Northern Ireland (GB); Derek John QUINN, Northern Ireland (GB)

(21) Appl. No.: 19/062,632

(22) Filed: Feb. 25, 2025

Related U.S. Application Data
(60) Provisional application No. 63/707,252, filed on Oct. 15, 2024.
(60) Provisional application No. 63/558,670, filed on Feb. 28, 2024.
(60) Provisional application No. 63/557,812, filed on Feb. 26, 2024.

(51) Int. Cl. C12P 7/6409; C07H 15/04; C12N 9/10; C12N 9/24

(52) U.S. Cl. CPC C12P 7/6409; C07H 15/04; C12N 9/1051; C12N 9/24

(57) ABSTRACT

A method of making glycomonolipids, comprising: (a) making glycomonolipids via a single catalytic step by contacting a hydroxy fatty acid with a glycosyl donor in the presence of an enzyme selected from i) a glycosyltransferase; and ii) glycosidases; (b) optionally isolating the made glycomonolipids; wherein the glycomonolipids have the following Formula 1:

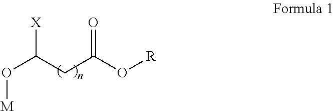

wherein n is from 0 to 15; wherein X is selected from an alkyl, aryl, heteroalkyl, heteroaryl, unsaturated alkenyl, and unsaturated heteroalkenyl, and combinations and stereoisomers thereof; and wherein X has a carbon chain length from 4 to 22; wherein M is selected from one or two of glucose, sulfoquinovose, fructose, galactose, ribose, maltose, xylose, rhamnose, sophorose, mannose, arabinose, fucose, and combinations and stereoisomers thereof; and wherein R is selected from —H, an alkyl selected from a straight-chained, branched or cyclic alkyl; a heteroalkyl; aryl; heteroaryl; hetero arylalkyl; arylalkyl; tauryl; and all possible stereoisomers and combinations thereof.

### CROSS REFERENCE TO RELATED APPLICATIONS

This application claims the benefit, under 35 U.S.C. § 119 (e), to U.S. Provisional Application Nos. 63/707,252 filed Oct. 15, 2024, 63/558670 filed Feb. 28, 2024, and 63/557812, filed Feb. 26, 2024, the entire disclosure of which is fully incorporated by reference herein.

### REFERENCE TO A SEQUENCE LISTING

The present application contains a Sequence Listing in computer-readable form. The computer-readable form is incorporated herein by reference.

### FIELD OF THE INVENTION

The present invention relates to cells and methods for producing glycomonolipids and more particularly relates to cells and methods for producing rhamnomonolipids.

### BACKGROUND OF THE INVENTION

In personal care products, surfactants may be used for cleansing, foaming, thickening, emulsifying, solubilizing, and antimicrobial effects, among other uses. But many surfactants that are commonly used are made from starting materials such as petrochemicals, while consumers are seeking more natural and milder materials in their products. Biosurfactants, which are surfactants of microbial origin, can provide improved efficacy without performance trade-offs.

### SUMMARY OF THE INVENTION

A method of making glycomonolipids, comprising: (a) making glycomonolipids via a single catalytic step by contacting a hydroxy fatty acid with a glycosyl donor in the presence of an enzyme selected from i) a glycosyltransferase; and ii) glycosidases; (b) optionally isolating the made glycomonolipids; wherein the glycomonolipids have the following Formula 1:

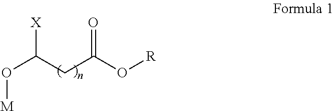

wherein n is from 0 to 15; wherein X is selected from an alkyl, aryl, heteroalkyl, heteroaryl, unsaturated alkenyl, and unsaturated heteroalkenyl, and combinations and stereoisomers thereof; and wherein X has a carbon chain length from 4 to 22; wherein M is selected from one or two of glucose, sulfoquinovose, fructose, galactose, ribose, maltose, xylose, rhamnose, sophorose, mannose, arabinose, fucose, and combinations and stereoisomers thereof; and wherein R is selected from —H, an alkyl selected from a straight-chained, branched or cyclic alkyl; a heteroalkyl; aryl; heteroaryl; hetero arylalkyl; arylalkyl; tauryl; and all possible stereoisomers and combinations thereof.

### BRIEF DESCRIPTION OF THE FIGURES

For a more complete understanding of the disclosure, reference should be made to the following detailed description and drawing Figure.

FIG. 1 is a rooted phylogenetic representation comparing 12 sequence IDs to Seq. ID No. 17 (root sequence) utilizing MUltiple Sequence Comparison by Log-Expectation (MUSCLE) alignment.

FIG. 2 shows exemplary rhamnolipid structures.

FIG. 3 is a chromatogram of formation of Rha-C10 by SEQ ID NO: 30 after 72 hours of reaction of rhamnose and 3-OH-decanoic acid in 75% glycerol at 15° C. Rha-C10 (RhamnolipidC10) formed was traced at 2.820 minutes retention time.

### DETAILED DESCRIPTION OF THE INVENTION

There is interest in non-traditional surfactants, such as glycolipid biosurfactants, as many palm-and petroleum-based surfactants suffer from sustainability, environmental, and socioeconomic challenges. Glycolipids consist of a diverse group of naturally occurring surfactant molecules with a range of structures (made up of a sugar polar group and a lipid group). The two main commercial classes of glycolipids are rhamnolipids (produced via bacterial plus fermentation) and sophorolipids (produced via yeast fermentation of mixed oil and sugar feed). In addition to being seen as environmentally friendly chemicals and enabling green credentialling, these materials have many other potential benefits such as mildness, moisturization, and cleaning effectiveness.

Many current commercial glycolipids have poor performance and high costs compared to current surfactants. Knowing the structures of certain high-performing rhamnolipids, the present inventors sought to produce optimized glycolipids. The inventors hypothesized that changing the molecular structure via simplification of the surfactant headgroup and elongation to a single chain length (making them more structurally similar to typical surfactants) would increase surfactancy. Production strategies involve a short-term, semi-synthetic fermentation approach, in which commercial rhamnolipids were leveraged as the feedstock and hydrolyzed to produce simplified monolipid glycolipid congener structures, (as described in U.S. Patent Nos. 63/557,812 and 63/558,670, Attorney Docket Nos. 16682P and 16682P2, which are incorporated by reference herein) or by hydrolyzing rhamnodilipids through the use of a hydrolase in a cell-free system or genetically engineered through recombinant DNA technology for heterologous expression in the host cell, (Attorney Docket No. 16700P, U.S. 63/641,630), also incorporated by reference herein.

The present invention involves methods of making biosurfactants via a one-step enzymatic glycosylation of a hydroxyl fatty acid. The biosurfactants may be made through use of a glycosyltransferase or glycosidase. In some embodiments, the glycosyltransferase or glycosidase may be engineered through recombinant DNA technology, such as by heterologous expression in a host cell. All of the inventive methods result in producing glycomonolipids [Formula 1], which may then offer commercial and consumer benefits.

The present invention provides methods and cells for making glycomonolipids. In certain embodiments, the method for producing glycomonolipids in a cell includes expressing in the cell one or more recombinant polypeptides that catalyze the glycosylation of a hydroxyl fatty acid, in some embodiments a glycosyltransferase or glycosidase, and culturing the cell under conditions suitable for producing the polypeptide, such that glycomonolipids, in some embodiments rhamnomonolipids, are produced.

In certain embodiments, a method for producing glycomonolipids in a cell is provided, the method including expressing in the cell a polypeptide that has glycotransferase or glycosidase activity; and culturing the cell under conditions suitable for producing the polypeptide, such that rhamnomonolipids are produced.

Also provided is a method for producing rhamnomonolipids in a cell, the rhamnomonolipids having a chain length from about 4 to 22 carbons. The method includes modifying the cell to increase carbon flow and culturing the cell under conditions suitable for carbon flow to be increased, such that-monolipids having a chain length from about 4 to about 22 carbons are produced.

Further provided is a Pseudomonas cell that produces rhamnomonolipids having a chain length from about 4 to 22 carbons.

The present invention provides a method of making glycomonolipids by glycosylation reaction of a hydroxy fatty acid with a glycosyl donor that is catalyzed by a glycosyltransferase or glycosidase; wherein the glycomonolipids have the following formula 1:

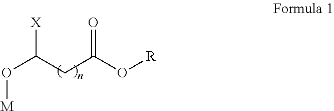

wherein n has a carbon chain length from 0 to 15 and X is selected from an alkyl, aryl, heteroalkyl, heteroaryl, unsaturated alkenyl, and unsaturated heteroalkenyl, and combinations and stereoisomers thereof; and has a carbon chain length from 4 to 22; wherein M is selected from one or two of glucose, sulfoquinovose, fructose, galactose, ribose, maltose, xylose, rhamnose, sophorose, mannose, arabinose, fucose, and combinations and stereoisomers thereof; and wherein R is selected from —H, an alkyl selected from a straight-chained, branched or cyclic alkyl; a heteroalkyl; aryl; heteroaryl; hetero arylalkyl; arylalkyl; tauryl, and all possible stereoisomers and combinations thereof. For any given embodiment, X, M, and R may be any combination thereof.

In some embodiments, the hydroxy fatty acid is a linear chain hydroxy fatty acid.

In some embodiments, in Formula 1, X may be selected from an alkyl and unsaturated alkenyl and have a carbon chain length from 4 to 22. In some embodiments, R may be selected from —H and an alkyl and have a straight-chain carbon length from 1 to 4.

In some embodiments, the glycotransferase comprises a polypeptide sequence having at least 60% identity, in some cases 75% identity, in some cases 90% identity, in some cases 95% identity, or in some cases 100% identity to any one of SEQ ID NOs: 1-12 or fragments thereof.

In some embodiments, the glycotransferase has amino acid substitutions compared to SEQ ID NO: 1. In some embodiments, SEQ ID NO: 1 may have the following amino acid substitutions: V25A, V31I, N49K, I82V, A83G, M163L, Q182R, A187P, T189V, L235F, L238P, G263S, T268A, S288P, S292A, I377V, E388D, S417Y for SEQ ID NO: 63, or V8I, V25A, V31I, N49K, I82V, E100Q, H119R, V124I, L161M, M163V, A167T, S179R, Q182R, A187P, T189A, H216R, G234S, L235F, P237E, L238P, E243D, G263S, T268A, S288P, S292T, S294A, S295T, H313Q, I337V, I377V, E388D, D398E, S401C, L406R, L411I, A412V, R413A, T414E, A415P, R416G, S417Y, A420R for SEQ ID NO: 64. In these embodiments, “V25A” represents where the valine (V, or Val) at position 25 in SEQ ID NO: 1 is substituted by an alanine (A, or Ala), in this cases, as found at position 25 in SEQ ID NO: 63.

In some embodiments, the glycosidase comprises a polypeptide sequence having at least 60% identity, in some cases 75% identity, in some cases 90% identity, in some cases 95% identity, and in some cases 100% identity to any one of SEQ ID NOs: 30-62 and 80, or fragments thereof. Preferably polypeptides sequence having at least 60% identity, in some cases 75% identity, in some cases 90% identity, in some cases 95% identity, and in some cases 100% identity to SEQ ID NOs: 30 or 80.

In some embodiments, the enzymes may be encapsulated and/or immobilized.

In some embodiments, the hydroxy fatty acid glycosylating enzyme, glycosyltransferase or glycosidase, may be made by being heterologously expressed in a host cell. The host cell may be a eukaryotic and/or a prokaryotic organism. The host cell may be, but is not limited to, at least one of Escherichia coli, Bacillus subtilis, Streptomyces coelicolor, Pseudomonas aeruginosa, Pichia pastoris, Saccharomyces cerevisiae, Kluyveromyces lactis, Aspergillus niger, Trichoderma reesei, Corynebacterium spp., Pseudomonas putida, Burkholderia thailandensis, Parabulkholderia spp., Acinetobacter spp., Alcanivorax spp., Antarctobacter spp., Bacillus spp., Burkholderia spp., Candida apicola, Cellulomonas cellulans, Cupriavidus necator, Enterobacterspp., Halomonas spp., Lactobacilli spp., Marinobacter spp., Myxococcus spp., Nocardioides spp., Pseudoalteromonas spp., Pseudomonas spp., Pseudoxanthomonas spp., Renibacterium salmoninarum, Rhodoccus spp., Rhodotorula bogoriensis, Tetragenococcus koreensis, Methylobacterium spp., and Methylorubrum spp.

In some embodiments, the glycomonolipids that are being made by the glycosyltransferase or glycosidase may be rhamnomonolipids.

In some embodiments, the glycosyl donor may comprise at least one rhamnose. The rhamnose may be, but is not limited to, a nucleoside monophosphate (NMP) nucleoside diphosphate (NDP) or a nucleoside triphosphate (NTP).

In some embodiments, the rhamnomonolipid produced may be selected from Rha-C8, Rha-C10, Rha-C12, Rha-C14, RhaRha-C8, RhaRha-C10, RhaRha-C12 and RhaRha-C14.

In some embodiments, the rhamnomonolipid produced may be selected from Rha-C10, Rha-C12 and Rha-C14. The rhamnomonolipid may be produced by reacting cell-free glycosyltransferase or glycosidase with a rhamnosyl donor and 3-hydroxyldecanoic acid or 3-hydoxylauric acid or 3-hydroxymyristic acid at a pH from 6 to 10; at a temperature from 25° C. to 42° C.; and under agitation in a buffered solution.

In some embodiments, the present invention provides a recombinant cell comprising the following:
(i) a glycosyltransferase gene comprising the SEQ ID NOs: 18-29 or (ii) glycosidase gene.

In some embodiments of the recombinant cell, the recombinant cell is Pseudomonas putida, Escherichia coli, Bacillus subtilis, Streptomyces coelicolor, Pseudomonas aeruginosa, Pichia pastoris, Saccharomyces cerevisiae, Kluyveromyces lactis, Aspergillus niger, Trichoderma reesei, Corynebacterium spp., Burkholderia thailandensis, Parabulkholderia spp., Acinetobacter spp., Alcanivorax spp., Antarctobacter spp., Bacillus spp., Burkholderia spp., Candida apicola, Cellulomonas cellulans, Cupriavidus necator, Enterobacter spp., Halomonas spp., Lactobacilli spp., Marinobacter spp., Myxococcus spp., Nocardioides spp., Pseudoalteromonas spp., Pseudomonas spp., Pseudoxanthomonas spp., Renibacterium salmoninarum, Rhodoccus spp., Rhodotorula bogoriensis Tetragenococcus koreensis, Methylobacterium spp., and Methylorubrum spp.

In some embodiments of the recombinant cell, the glycosyltransferase may comprise a polypeptide sequence having at least 60%, in some embodiments at least 70%, or 75%, 80%, or 90%, 95%, or 100% identity to any one of SEQ ID NOs: 1-12. In other embodiments of the recombinant cell, the glycosidase may comprise a polypeptide sequence having at least 60%, in some embodiments at least 70%, or 75%, or 80%, or 90%, or 95%, or 100% identity to any one of SEQ ID NOs: 30-62 and 80.

In other embodiments, the present invention provides a method of making rhamnomonolipids in a recombinant cell, said method comprising the following steps:
a) culturing the recombinant cell to generate rhamnomonolipids; and
b) optionally isolating the resulting rhamnomonolipids;
wherein said recombinant cell comprises (i) a glycosyltransferase comprising the SEQ ID NOs: 1-12 or (ii) glycosidase comprising the SEQ ID NOs: 30-62, or 80. In some embodiments of the method of making rhamnomonolipids in a recombinant cell, the glycosyltransferase may comprise a polypeptide sequence having at least 60% identity to SEQ ID NOs: 1-12 or fragments thereof, in some embodiments at least 70% identity, in some embodiments at least 80% identity, in some embodiments at least 90% identity, in some embodiments at least 95% identity, and in some embodiments 100% identity. In some embodiments of the method of making rhamnomonolipids in a recombinant cell, the glucosidase may comprise a polypeptide sequence having at least 60% identity to SEQ ID NOs: 30-62, or 80 in some embodiments at least 70% identity, in some embodiments at least 80% identity, in some embodiments at least 90% identity, in some embodiments at least 95% identity, and in embodiments 100% identity.

In some embodiments, the present invention provides a method of producing glycomonolipids, wherein glycomonolipids are generated through growth of an engineered cell by either:
A.) constitutive growth from a carbon source; (such as feedstocks or sole carbon sources) to yield glycomonolipids;
B.) grown cells from a carbon source; (such as feedstocks or sole carbon) are then lysed to generate a composite of proteins to react starting materials to lead to glycomonolipids in vitro or ex vivo;
comprising the following steps:
a) Contacting a fatty acid with a glycosyl donor in the presence of a glycosyltransferase or glycosidase
b) optionally isolating the glycomonolipids;

wherein the glycomonolipids have the following formula 1:

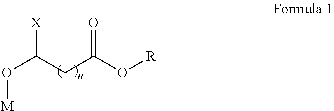

wherein n=0-15; X is selected from an alkyl, aryl, heteroalkyl, heteroaryl, unsaturated alkenyl, and unsaturated heteroalkenyl; and has a carbon chain length from 4 to 22;
wherein M is selected from one or two of glucose, sulfoquinovose, fructose, galactose, ribose, maltose, xylose, rhamnose, sophorose, mannose, arabinose, fucose, and combinations and stereoisomers thereof; and wherein R is selected from —H, an alkyl selected from a straight-chained, branched or cyclic alkyl; a heteroalkyl; aryl; heteroaryl; hetero arylalkyl; arylalkyl; tauryl, and all possible stereoisomers thereof.

Reference within the specification to “embodiment(s)” or the like means that a particular material, feature, structure and/or characteristic described in connection with the embodiment is included in at least one embodiment, optionally a number of embodiments, but it does not mean that all embodiments incorporate the material, feature, structure, and/or characteristic described. Furthermore, materials, features, structures and/or characteristics may be combined in any suitable manner across different embodiments, and materials, features, structures and/or characteristics may be omitted or substituted from what is described. Thus, embodiments and aspects described herein may comprise or be combinable with elements or components of other embodiments and/or aspects despite not being expressly exemplified in combination, unless otherwise stated or an incompatibility is stated.

All ingredient percentages described herein are by weight of the cosmetic composition, unless specifically stated otherwise, and may be designated as “wt %.” All ratios are weight ratios, unless specifically stated otherwise. All ranges are inclusive and combinable. The number of significant digits conveys neither a limitation on the indicated amounts nor on the accuracy of the measurements. All numerical amounts are understood to be modified by the word “about” unless otherwise specifically indicated. Unless otherwise indicated, all measurements are understood to be made at approximately 25° C. and at ambient conditions, where “ambient conditions” means conditions under about 1 atmosphere of pressure and at about 50% relative humidity. All numeric ranges are inclusive of narrower ranges, and delineated upper and lower range limits are interchangeable to create further ranges not explicitly delineated.

The compositions of the present invention can comprise, consist essentially of, or consist of, the essential components as well as optional ingredients described herein. As used herein, “consisting essentially of” means that the composition or component may include additional ingredients, but only if the additional ingredients do not materially alter the basic and novel characteristics of the claimed compositions or methods. As used in the description and the appended claims, the singular forms “a,” “an,” and “the” are intended to include the plural forms as well, unless the context clearly indicates otherwise.

### Definitions

When used in the context of a chemical group: “hydrogen” means —H; “hydroxy” means —OH; “oxo” means ═O; “carbonyl” means —C(═O)—; “carboxy” and “carboxylate” mean —C(═O)OH (also written as —COOH or —CO2H) or a deprotonated form thereof; “amino” means —NH2; “hydroxyamino” means —NHOH; “nitro” means —NO2; “imino” means ═NH; “amine oxide” means N⁺O⁻ where N has three covalent bonds to atoms other than O; “hydroxamic” or “hydroxamate” means-C (O) NHOH or a deprotonated form thereof.

The term “cation” refers to an atom, molecule, or a chemical group with a net positive charge including single and multiple charged species. Cations can be individual atoms such as metals, non-limiting examples include Na⁺ or Ca⁺², individual molecules, non-limiting examples include (CH₃)₄N⁺, or a chemical group, non-limiting examples include —N(CH₃)₃⁺. The term “amine cation” refers to a particular molecular cation, of the form NR₄⁺ where the four substituting R moieties can be independently selected from H and alkyl, non-limiting examples include NH₄⁺ (ammonium), CH₃NH₃⁺ (methylammonium), CH₃CH₂NH₃⁺ (ethylammonium), (CH₃)₂NH₂⁺ (dimethylammonium), (CH₃)₃NH⁺ (trimethyl ammonium), and (CH₃)₄N⁺ (tetramethylammonium). In some embodiments, a cation may be selected from Na+, K+, Li+, Cs+, +NH₃R2; +NH₂R2R3; +NHR2R3R4, +NR2R3R4R5 wherein R2, R3, R4, and R5 are each independently selected from an alkyl, branched alkyl, and cyclic alkyl.

The term “anion” refers to an atom, molecule, or chemical group with a net negative charge including single and multiply charged species. Anions can be individual atoms, for example but not limited to halides F⁻, Cl⁻, Br⁻, individual molecules, non-limiting examples include CO₃⁻², H₂PO₄⁻, HPO₄⁻², PO₄⁻³, HSO₄⁻, SO₄⁻², or a chemical group, non-limiting examples include sulfate, phosphate, sulfonate, phosphonate, phosphinate, sulfonate, mercapto, carboxylate, amine oxide, hydroxamate and hydroxyl amino. Deprotonated forms of previously defined chemical groups are considered anionic groups if the removal of the proton results in a net negative charge. In solutions, chemical groups can lose a proton and become anionic as a function of pH according to the Henderson-Hasselbach equation (pH=pKa+log₁₀([A⁻]/[HA]; where [HA] is the molar concentration of an undissociated acid and [A⁻ ] is the molar concentration of this acid's conjugate base). When the pH of the solution equals the pKa value of functional group, 50% of the functional group will be anionic, while the remaining 50% will have a proton. Typically, a functional group in solution can be considered anionic if the pH is at or above the pKa of the functional group.

The term “salt” or “salts” refers to the charge neutral combination of one or more anions and cations. For example, when R is denoted as a salt for the carboxylate group, —COOR, it is understood that the carboxylate (—COO—) is an anion with a negative charge −1, and that the R is a cation with a positive charge of +1 to form a charge neutral entity with one anion of charge −1, or R is a cation with a positive charge of +2 to form a charge neutral entity with two anions both of −1 charge.

The term “saturated” as used herein means the chemical compound or group so modified has no carbon-carbon double and no carbon-carbon triple bonds, except as noted below. In the case of substituted versions of saturated chemical groups, one or more carbon oxygen double bond or a carbon nitrogen double bond may be present. When such a bond is present, then carbon-carbon double bonds that may occur as part of keto-enol tautomerism or imine/enamine tautomerism are not precluded.

The term “aliphatic” when used without the “substituted” modifier signifies that the chemical compound/group so modified is an acyclic or cyclic, but non-aromatic hydrocarbon chemical compound or group. In aliphatic chemical compounds/groups, the carbon atoms can be joined together in straight chains, branched chains, or non-aromatic rings (alicyclic). Aliphatic chemical compounds/groups can be saturated, that is joined by single bonds (alkanes/alkyl), or unsaturated, with one or more double bonds (alkenes/alkenyl), or with one or more triple bonds (alkynes/alkynyl).

The term “alkyl” when used without the “substituted” modifier refers to a monovalent saturated aliphatic group with a carbon atom as the point of attachment, a linear or branched, cyclo, cyclic, or acyclic structure, and no atoms other than carbon and hydrogen. Thus, as used herein cycloalkyl is a subset of alkyl, with the carbon atom that forms the point of attachment also being a member of one or more non-aromatic ring structures wherein the cycloalkyl group consists of no atoms other than carbon and hydrogen. As used herein, the term does not preclude the presence of one or more alkyl groups (carbon number limitation permitting) attached to the ring or ring system. The groups —CH₃ (Me), —CH₂CH₃ (Et), —CH₂CH₂CH₃ (n-Pr or propyl), —CH(CH₃)₂ (i-Pr, ′Pr, or isopropyl), —CH(CH₂)₂ (cyclopropyl), —CH₂CH₂CH₂CH₃ (n-Bu), —CH(CH₃)CH₂CH₃ (sec-butyl), —CH₂CH(CH₃)₂ (isobutyl), —C(CH₃)₃ (tertbutyl, t-butyl, t-Bu, or tBu), —CH₂C(CH₃)₃ (neo-pentyl), cyclobutyl, cyclopentyl, cyclohexyl, and cyclohexylmethyl are non-limiting examples of alkyl groups. The term “alkanediyl” when used without the “substituted” modifier refers to a divalent saturated aliphatic group, with one or two saturated carbon atom(s) as the point(s) of attachment, a linear or branched, cyclo, cyclic or acyclic structure, no carbon-carbon double or triple bonds, and no atoms other than carbon and hydrogen. The groups, —CH₂-(methylene), —CH₂CH₂—, —CH₂C(CH₃)₂CH₂—, and —CH₂CH₂CH₂— are non-limiting examples of alkanediyl groups. The term “alkylidene” when used without the “substituted” modifier refers to the divalent group ═CRR′ in which R and R′ are independently hydrogen, alkyl, or R and R′ are taken together to represent an alkanediyl having at least two carbon atoms. Non-limiting examples of alkylidene groups include: ═CH₂, ═CH(CH₂CH₃), and ═C(CH₃)₂. An “alkane” refers to the compound H-R, wherein R is alkyl as this term is defined above.

When any of these terms is used with the “substituted” modifier one or more hydrogen atom has been independently replaced by —OH, —F, —Cl, —Br, —I, —NH₂, —NO₂, —CO₂H, —CO₂CH₃, —CN, —SH, —OCH₃, —OCH₂CH₃, —C(O)CH₃, —NHCH₃, —NHCH₂CH₃, —N(CH₃)₂, —C(O)NH₂, —OC(O)CH₃, —S(O)₂NH₂, —P(O)(OH)₂, —P(O)(OH)OP(O)(OH)₂, —OP(O)(OH)₂, —OP(O)(OH)OP(O)(OH)₂, —S(O)₂(OH), or —OS(O)₂(OH). The following groups are non-limiting examples of substituted alkyl groups: —CH₂OH, —CH₂Cl, —CF₃, —CH₂CN, —CH₂C(O)OH, —CH₂C(O)OCH₃, —CH₂C(O)NH₂, —CH₂C(O)CH₃, —CH₂OCH₃, —CH₂OC(O)CH₃, —CH₂NH₂, —CH₂N(CH₃)₂, —CH₂CH₂Cl, —CH₂P(O)(OH)₂, —CH₂P(O)(OH)OP(O)(OH)₂, —CH₂S(O)₂(OH), and —CH₂OS(O)₂(OH). The term “haloalkyl” is a subset of substituted alkyl, in which one or more hydrogen atoms has been substituted with a halo group and no other atoms aside from carbon, hydrogen and halogen are present. The group, —CH₂Cl is a non-limiting example of a haloalkyl. The term “fluoroalkyl” is a subset of substituted alkyl, in which one or more hydrogen has been substituted with a fluoro group and no other atoms aside from carbon, hydrogen and fluorine are present. The groups, —CH₂F, —CF₃, and —CH₂CF₃ are non-limiting examples of fluoroalkyl groups.

The term “alkenyl” when used without the “substituted” modifier refers to a monovalent unsaturated aliphatic group with a carbon atom as the point of attachment, a linear or branched, cyclo, cyclic or acyclic structure, at least one nonaromatic carbon-carbon double bond, no carbon-carbon triple bonds, and no atoms other than carbon and hydrogen. Non-limiting examples of alkenyl groups include: —CH═CH₂ (vinyl), —C(CH₃)═CH₂ (methyl-vinyl), —CH═CHCH₃, —CH═CHCH₂CH₃, —CH₂CH═CH₂ (allyl), —CH₂CH═CHCH₃, and —CH═CHCH═CH₂. The term “alkenediyl” when used without the “substituted” modifier refers to a divalent unsaturated aliphatic group, with two carbon atoms as points of attachment, a linear or branched, cyclo, cyclic or acyclic structure, at least one nonaromatic carbon-carbon double bond, no carbon-carbon triple bonds, and no atoms other than carbon and hydrogen. The groups, >C═CH₂ (vinylidine), —CH═CH—, —CH═C(CH₃)CH₂—, and —CH═CHCH₂—, are non-limiting examples of alkenediyl groups. It is noted that while the alkenediyl group is aliphatic, once connected at both ends, this group is not precluded from forming part of an aromatic structure. The terms “alkene” or “olefin” are synonymous and refer to a compound having the formula H—R, wherein R is alkenyl as this term is defined above.

The term “aryl” when used without the “substituted” modifier refers to a functional group derived from a simple aromatic ring compound where the point of attachment is a carbon atom on the aromatic ring. An aromatic ring is a hydrocarbon that has a cyclic structure and a delocalized electron system. An aryl group is formed by removing one hydrogen atom from the ring. The name of the aryl group is based on the name of the aromatic ring with the-yl suffix, such as phenyl, naphthyl, indolyl, etc. Aryl groups may be substituted with alkyl and/or heteroalkyl chains and may have one or more heteroatoms within the aryl ring.

The term “arylalkyl” refers to an aryl group attached to an alkanediyl group where the point of attachment is on the alkanediyl group.

Tauryl/taurate is defined as aminoethyl sulphonyl.

Hetero is defined as an atom other than carbon, including but not exclusive to, nitrogen (N), oxygen (O), or sulfur (S). The heteroatom may be attached in a linear or branched alkyl chain or also may be attached to a non-aromatic or aromatic ring, either as part of the ring, or adjacent to it as a substituent. There may be more than one heteroatom in the alkyl chain or ring.

Unsaturated is defined as a hydrocarbon chain possessing, but not limited, at least one carbon-carbon double bond (C═C). The unsaturated chain may possess one double bond (alkenyl), two double bonds (dienyl), multiple double bonds (polyenyl), and/or a carbon-carbon triple bond (acetylenic). The unsaturated bonds may be adjacent (conjugated) relative to each other or separated by additional carbon atoms in the chain.

A “monomer molecule” is defined by the International Union of Pure and Applied Chemistry (IUPAC) as “A molecule which can undergo polymerization thereby contributing constitutional units to the essential structure of a macromolecule.” A polymer is a macromolecule.

“Isolation” as used in reference to removal of enzyme from solution either before or after reaction with glycolipids.

“Immobilization” as used in reference to fixation of glycosyltransferase or glycosidase to a solid support so that it is removed from solution or to stabilize the enzyme. Some examples of solid supports among several others known in the art include silica, chitosan and bacterial spores.

“Treat” or “treating” as used in reference to a composition, means to add or apply a material to the composition.

“About” modifies a particular value by referring to a range of plus or minus 20% or less of the stated value (e.g., plus or minus 15% or less, 10% or less, or even 5% or less).

“Apply” or “application,” as used in reference to a composition, means to apply or spread the composition onto a human keratinous surface such as the skin or hair.

“Charge density” (“CD”) means the ratio of positive charges on a polymer to the molecular weight of the polymer.

“Personal care composition” means a product, which in the ordinary course of usage is applied to or contacted with a body surface to provide a beneficial effect. Body surface includes skin, for example dermal or mucosal; body surface also includes structures associated with the body surface for example hair, teeth, or nails. Examples of personal care compositions include a product applied to a human body for improving appearance, cleansing, and odor control or general aesthetics. Non-limiting examples of personal care compositions include oral care compositions, such as, dentifrice, mouth rinse, mousse, foam, mouth spray, lozenge, chewable tablet, chewing gum, tooth whitening strips, floss and floss coatings, breath freshening dissolvable strips, denture care product, denture adhesive product; after shave gels and creams, pre-shave preparations, shaving gels, creams, or foams, moisturizers and lotions; cough and cold compositions, gels, gel caps, and throat sprays; leave-on skin lotions and creams, shampoos, body washes, body rubs, such as Vicks VapoRub; hair conditioners, hair dyeing and bleaching compositions, mousses, shower gels, bar soaps, antiperspirants, deodorants, depilatories, lipsticks, foundations, mascara, sunless tanners and sunscreen lotions; feminine care compositions, such as lotions and lotion compositions directed towards absorbent articles; baby care compositions directed towards absorbent or disposable articles; and oral cleaning compositions for animals, such as dogs and cats. Further non-limiting examples include hand soaps, hand sanitizers, body washes, shower gels, shampoos, body lotions, feminine care products, foot care products, deodorants, pet care products and combinations thereof. Further non-limiting examples include a wipe product suitable for personal care use and household cleaning; a toilet tissue; a towel for hand drying, household drying and household cleaning; a facial tissue; a skin care composition; a first aid or surgical antiseptic; a diaper; a feminine napkin; and combinations thereof.

The term “detergent composition” refers to a composition or formulation designed for cleaning soiled surfaces. Such compositions include but are not limited to, dishwashing compositions, laundry detergent compositions, fabric softening compositions, fabric enhancing compositions, fabric freshening compositions, laundry pre-wash, laundry pretreat, laundry additives, spray products, dry cleaning agent or composition, laundry rinse additive, wash additive, post-rinse fabric treatment, ironing aid, hard surface cleaning compositions, unit dose formulation, delayed delivery formulation, detergent contained on or in a porous substrate or nonwoven sheet, and other suitable forms that may be apparent to one skilled in the art in view of the teachings herein. Such compositions may be used as a pre-cleaning treatment, a post-cleaning treatment, or may be added during the rinse or wash cycle of the cleaning process. The detergent compositions may have a form selected from liquid, powder, single-phase or multi-phase unit dose or pouch form, tablet, gel, paste, bar, or flake. Preferably the composition is for manual washing. The detergent composition of the present invention may be a dishwashing detergent. The composition may be in the form of a liquid. Further non-limiting examples include hard surface cleaners, deodorizers, fabric care compositions, fabric cleaning compositions, manual dish detergents, automatic dish detergents, floor waxes, kitchen cleaners, bathroom cleaners and combinations thereof.

“Cleansing composition” refers to a personal care composition or product intended for use in cleaning a bodily surface such as skin or hair. Some non-limiting examples of cleansing compositions are shampoos, conditioners, conditioning shampoos, shower gels, liquid hand cleansers, facial cleansers, and the like.

“Cosmetic agent” means any substance, as well any component thereof, intended to be rubbed, poured, sprinkled, sprayed, introduced into, or otherwise applied to a mammalian body or any part thereof to provide a cosmetic effect. Cosmetic agents may include substances that are Generally Recognized as Safe (GRAS) by the US Food and Drug Administration and food additives.

“Gel network phase” or “dispersed gel network phase” refers to a lamellar or vesicular solid crystalline phase that includes at least one fatty alcohol, at least one gel network surfactant, and a liquid carrier. The lamellar or vesicular phase can be formed of alternating layers with one layer including the fatty alcohol and the gel network surfactant and the other layer formed of the liquid carrier.

“Solid crystalline” refers to the crystalline structure of the lamellar or vesicular phase at ambient temperatures caused by the phase being below its melt transition temperature. For example, the melt transition temperature of the lamellar or vesicular phase may be about 30° C. or more (i.e., slightly above about room temperature). The melt transition temperature can be measured through differential scanning calorimetry, which is conventional measurement method known to those skilled in the art.

“Suitable for application to human hair” means that the personal care composition or components thereof, are acceptable for use in contact with human hair and the scalp and skin without undue toxicity, incompatibility, instability, allergic response, and the like.

“Substantially free of” means a composition or ingredient comprises less than 3% of a subject material, by weight of the composition or ingredient (e.g., less than 2%, less than 1% or even less than 0.5%). “Free of” means a composition or ingredient contains 0% of a subject material.

“Sulfated surfactants” means surfactants that contain a sulfate moiety. Some non-limiting examples of sulfated surfactants are sodium lauryl sulfate, sodium laureth sulfate, ammonium lauryl sulfate, and ammonium laureth sulfate. “Sulfate-free surfactant” refers to a surfactant that has no sulfate moieties.

The above definitions supersede any conflicting definition in any reference that is incorporated by reference herein. The fact that certain terms are defined, however, should not be considered as indicative that any term that is undefined is indefinite. Rather, all terms used are believed to describe the invention in terms such that one of ordinary skill can appreciate the scope and practice the present invention.

This is not an exhaustive list of sugars, and there are many other sugars with different structures: Glucose is a monosaccharide and is the primary source of energy in living organisms. Its chemical formula is C6H12O6. Fructose is another monosaccharide and is commonly found in fruits and honey. Its chemical formula is also C6H12O6. Galactose is a monosaccharide that is found in milk and dairy products. Its chemical formula is C6H12O6. Sucrose, also known as table sugar, is a disaccharide composed of glucose and fructose. Its chemical formula is C12H22O11. Lactose is a disaccharide found in milk and dairy products. It consists of glucose and galactose. Its chemical formula is C12H22O11. Maltose is a disaccharide formed by the combination of two glucose molecules. It is commonly found in germinating grains. Its chemical formula is C12H22O11. Ribose is a monosaccharide that is an essential component of RNA (ribonucleic acid) and plays a role in energy metabolism. Its chemical formula is C5H10O5. Deoxyribose is a monosaccharide that is a component of DNA (deoxyribonucleic acid). It is similar to ribose but lacks one oxygen atom. Its chemical formula is C5H10O4.

### Rhamnolipid Surfactant

Rhamnose (Rha, Rham), formula illustrated below, is a naturally occurring deoxy sugar. It can be classified as either a methyl-pentose or a 6-deoxy-hexose.

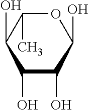

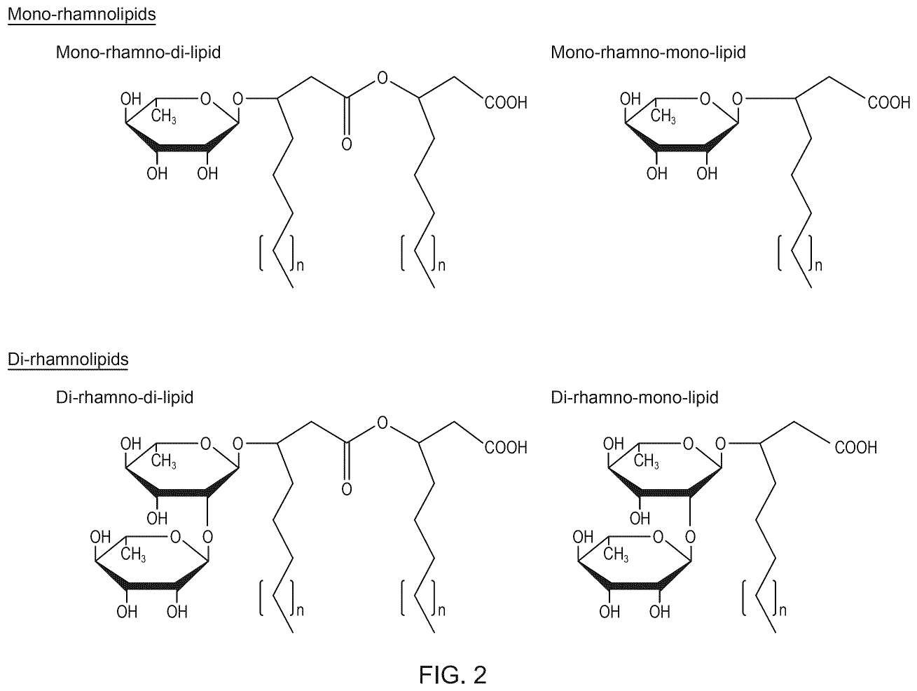

Rhamnolipids are a class of glycolipid that may be used as bacterial surfactants. They have a glycosyl head group, in this case a rhamnose moiety, and an acid fatty tail. There are two main classes of rhamnolipids: monorhamnolipids and di-rhamnolipids, which consist of one or two rhamnose groups respectively. And then rhamnomonolipids and rhamnodilipids may each have either one or two lipids, for example, the combinations as shown in FIG. 2.

The rhamnolipid surfactants herein may be produced by microorganisms (e.g., Pseudomonas aeruginosa, Pseudomonas putida, Pseudomonas chlororaphis). The rhamnolipid surfactant(s) may provide a cleaning benefit due to their amphiphilic nature, which allows the surfactants to break up, and form micelles around, oil and other contaminants. The “entrapped” contaminant can then be rinsed off more easily with water. A description of various types of rhamnolipids is disclosed in EP2410039. Certain methods of making, extracting, and blending naturally produced rhamnolipids are known in the art. The present invention includes making of mono-rhamno-mono-lipid biosurfactants.

### Glycosylation of Hydroxy Fatty Acids

In an embodiment of the present invention, 3-hydroxy fatty acids are glycosylated by an enzyme resulting in rhamnomonolipids (Rha-CX). Wherein Rha refers to the single rhamnose group, C is defined as the monolipid and X the carbon chain length. For example, Rha-C10 is a rhamnomonolipid containing ten carbons in its chain.

The glycosylating enzyme may be selected from, but not limited to, the group consisting of glycosyltransferases or glycosidases.

Glycosyltransferases (EC 2.4.1): In the context of the current application, a glycosyltransferase is an enzyme that can catalyze the transfer of a sugar moiety to an acceptor molecule. Glycotransferases include, but are not limited to, the group consisting of rhamnosyltransferases and glucosyltransferases.

The present invention includes rhamnosyltransferases (RhlB and RhlC) from bacteria species. RhlB adds one rhamnose sugar to a lipid and RhlC adds a second rhamnose.

Glycosidases (EC 3.2.1): these catalyze the hydrolysis of glycosidic bond. These enzymes can also perform reverse reaction where a glycosidic bond is reformed.

The present invention also includes variants of enzymes. Variants of enzymes, as used herein, include a sequence resulting when a wild-type protein of the respective protein is modified by, or at, one or more amino acids (for example 1, 2, 5 or 10 amino acids). The invention also includes variants in the form of truncated forms derived from a wild-type enzyme, such as a protein with a truncated N-terminus or a truncated C-terminus. Some enzymes may include an N-terminal signal peptide that is likely removed upon secretion by the cell. The present invention includes variants without the N-terminal signal peptide.

Bioinformatic tools, such as SignalP ver 4.1 (Petersen T N., Brunak S., von Heijne G. and Nielsen H. (2011), Nature Methods, 8:785-786), can be used to predict the existence and length of such signal peptides. The invention also includes variants derived by adding an extra amino acid sequence to a wild-type protein, such as for example, an N-terminal tag, a C-terminal tag or an insertion in the middle of the protein sequence. Non-limiting examples of tags are maltose binding protein (MBP) tag, glutathione S-transferase (GST) tag, thioredoxin (Trx) tag, 6× Histidine-tag, small peptide flag (DYKDDDDK) tag and any other tags known by those skilled in art. Tags can be used to improve solubility and expression levels during fermentation or as a handle for enzyme purification.

It is important that variants of enzymes retain and preferably improve the ability of the wild-type protein to catalyze the conversion of the unsaturated fatty acids. Some performance drop in a given property of variants may of course be tolerated, but the variants should retain and preferably improve suitable properties for the relevant application for which they are intended. Screening of variants of one of the wild-types can be used to identify whether they retain and preferably improve appropriate properties.

The variants may have “conservative” substitutions. Suitable examples of conservative substitution includes one conservative substitution in the enzyme, such as a conservative substitution in SEQ ID NO: 1.

An enzyme can be produced to contain one or more conservative substitutions by manipulating the nucleotide sequence that encodes that enzyme using, for example, standard procedures such as site-directed mutagenesis or PCR.

Examples of amino acids which may be substituted for an original amino acid in an enzyme and which are regarded as conservative substitutions include: Ser for Ala; Lys for Arg; Gln or His for Asn; Glu for Asp; Asn for Gln; Asp for Glu; Pro for Gly; Asn or Gln for His; Leu or Val for Ile; Ile or Val for Leu; Arg or Gln for Lys; Leu or Ile for Met; Met, Leu or Tyr for Phe; Thr for Ser; Ser for Thr; Tyr for Trp; Trp or Phe for Tyr; and Ile or Leu for Val. Conserved vs. non-conserved may also be functionally similar vs. dissimilar.

The variants may also have “non-conservative” substitutions, where the resulting amino acid has different properties from the original. Examples of amino acids which may be substituted for an original amino acid in an enzyme and which are regarded as non-conservative substitutions includes: Tyr for Ala; Asp for Gly and Lys for Pro.

A variant includes a “modified enzyme” or a “mutant enzyme” which encompasses proteins having at least one substitution, insertion, and/or deletion of an amino acid. Suitable examples of a modified enzyme include modifications such as modifications to SEQ ID NO: 1. An enzyme of the invention may therefore include 1, 2, 3, 4, 5, 6, 7, 8, 9 or 10 or more amino acid modifications (selected from substitutions, insertions, deletions and combinations thereof) of SEQ ID NO: 1, such as those modifications found in SEQ ID NOs: 63-70.

Compared to SEQ ID NO: 1, modifications found in SEQ ID NO: 63 are substitutions of V25A, V31I, N49K, I82V, A83G, M163L, Q182R, A187P, T189V, L235F, L238P, G263S, T268A, S288P, S292A, I377V, E388D, S417Y; modifications found in SEQ ID NO: 64 are substitutions of V8I, V25A, V31I, N49K, I82V, E100Q, H119R, V124I, L161M, M163V, A167T, S179R, Q182R, A187P, T189A, H216R, G234S, L235F, P237E, L238P, E243D, G263S, T268A, S288P, S292T, S294A, S295T, H313Q, I337V, I377V, E388D, D398E, S401C, L406R, L411I, A412V, R413A, T414E, A415P, R416G, S417Y, and A420R; modifications found in SEQ ID NO: 65 are substitutions of V8I, V25A, V31I, A146T, M163V, Q182R, A187P, E209D, L235F, L238V, E243D, G263S, S288P, S292T, A315S, L359V, T372A, I377V, E388D, and D398E; modifications found in SEQ ID NO: 66 are substitutions of V8I, V25A, V31I, E61A, E100Q, V124I, A136P, A146T, V156L, A167T, Q182R, A187P, T189V, L235F, P237Q, L238P, G263S, T268A, S288P, S292T, A315S, M330I, I377V, D398E, S401C, and R416K, modifications found in SEQ ID NO: 67 are substitutions of V8I, V25A, V31I, N49K, E61A, I82V, A146T, M163F, Q182R, A187P, L235F, P237Q, L238T, E243D, G263S, T268A, S288P, S292T, A315S, L359V, T372A, I377V, E388D, I394M, S401C, R416T, and A419V, modifications found in SEQ ID NO: 68 are substitutions of V8I, V25A, V31I, N49K, D60Q, E61A, S84G, E100D, A126G, F133I, A136P, S140P, T141I, V156L, K157R, M163V, R166H, A167T, F168V, V172L, A174K, Q182R, A187P, T189V, E209D, H216Q, L235F, P237E, L238P, N262D, G263A, T268A, V271A, S288P, S292A, V293L, E296D, S314C, A315S, M330F, I377V, T392K, I394M, S401C, and A419V; modifications found in SEQ ID NO: 69 are substitutions of V8I, V25A, V31I, N49K, R56A, D60E, E61A, I82V, Q95A, V124I, A138S, T141V, A146T, M163L, S179R, Q182R, A187P, R202G, H216S, L235F, P237Q, L238T, E243D, N247S, T268A, L276A, S286R, S288P, S292T, S294A, S295A, E296A, R298Q, I337V, L359V, V361L, F362S, T372A, I377V, Q381P, S386N, E388A, T392A, S401C, I408VLS, A415K, R416T, and A419V, and modifications found in SEQ ID NO: 70 are substitutions of V8I, V25A, E61A, A136P, S140P, V156L, K157R, Q182R, T189V, E209D, L235F, L238P, T268A, V271A, S288P, S292A, V293L, S314C, I377V, and T392K.

Compared to SEQ ID NO: 1, preferred modifications are substitutions of V25A, V31I, N49K, I82V, Q182R, A187P, L235F, L238P, G263S, T268A, S288P, I377V, E388D, and S417Y, which are found in SEQ ID NO: 63 and in SEQ ID NO: 64. More preferred substitutions are V25A, V31I, Q182R, A187P, L235F, G263S, S288P, I377V, and E388D, which are found in SEQ ID NO: 63, SEQ ID NO: 64, and in SEQ ID NO: 65. Most preferred substitutions are V25A, V31I, Q182R, A187P, L235F, S288P, and I377V, which are found in SEQ ID NO: 63, SEQ ID NO: 64, SEQ ID NO: 65, SEQ ID NO: 66, and in SEQ ID NO: 68.

Enzymes can be modified by a variety of chemical techniques to produce derivatives having essentially the same or preferably improved activity as the unmodified enzymes, and optionally having other desirable properties. For example, carboxylic acid groups of the protein, whether carboxyl-terminal or side chain, may be provided in the form of a salt of a pharmaceutically-acceptable cation or esterified, for example to form a C1-C6 alkyl ester, or converted to an amide, for example of formula CONR1R2 wherein R1 and R2 are each independently H or C1-C6 alkyl, or combined to form a heterocyclic ring, such as a 5- or 6-membered ring. Amino groups of the enzyme, whether amino-terminal or side chain, may be in the form of a pharmaceutically-acceptable acid addition salt, such as the HCl, HBr, acetic, benzoic, toluene sulfonic, maleic, tartaric and other organic salts, or may be modified to C1-C20 alkyl or dialkyl amino or further converted to an amide. Hydroxyl groups of the protein side chains may be converted to alkoxy or ester groups, for example C1-C20 alkoxy or C1-C20 alkyl ester, using well-recognized techniques. Phenyl and phenolic rings of the protein side chains may be substituted with one or more halogen atoms, such as F, CI, Br or I, or with C1-C20 alkyl, C1-C20 alkoxy, carboxylic acids and esters thereof, or amides of such carboxylic acids. Methylene groups of the protein side chains can be extended to homologous C2-C4 alkylenes. Thiols can be protected with any one of a number of well-recognized protecting groups, such as acetamide groups. Those skilled in the art will also recognize methods for introducing cyclic structures into the proteins of this disclosure to select and provide conformational constraints to the structure that result in enhanced stability.

Amino acid identity percentages were calculated from alignments generated via MUltiple Sequence Comparison by Log-Expectation (MUSCLE) version 5.1 (Edgar, RC (2021), MUSCLE v5 enables improved estimates of phylogenetic tree confidence by ensemble bootstrapping, bioRxiv 2021 Jun. 20) with the Super5 algorithm.

For enzyme sequence comparison the following settings can be used: Alignment algorithm: Needleman and Wunsch, J. Mol. Biol. 1970, 48:443-453. As a comparison matrix for amino acid similarity the Blosum62 matrix is used (Henikoff S. and Henikoff J. G., P.N.A.S. USA 1992, 89:10915-10919). The following gap scoring parameters are used: Gap penalty: 12, gap length penalty: 2, no penalty for end gaps.

In some embodiments of the present invention, the method of making glycomonolipids by a single catalytic step by contacting a hydroxy fatty acid with a glycosyl donor in the presence of an enzyme may be done with an enzyme that may be a glycosyltransferase that comprises at least one amino acid mutation selected from M1A, H2R, V3I, I4L, I4V, I5L, I5V, T6I, A7P, V8I, F17L, V18L, V18L, V18I, I20L, T23A, T23S, T23S, T23A, T23E, V25A, A26S, V31I, T32S, F33V, F33M, C34A, A35T, A35V, S36Y, S36N, S36F, A37P, A37EW, A37EF, A37PA, A37E, A37G, A37P, A38V, A38G, A38Y, A38R, F39L, F39A, A40K, A40R, A40P, A40E, A40S, P41E, P41S, P41A, L42S, L42A, I43A, I43V, I43L, E44D, E44G, R45L, R45G, R45E, C46S, C46A, C46P, C46R, G47A, F48M, F48L, N49S, N49A, N49E, N49T, N49G, N49G, N49K, N49E, N49R, N49S, F50V, F50L, L51I, L51V, L51V, L51V, P52A, L53V, L53R, L53I, G54T, T55E, T55S, T55P, R56A, R56A, R56A, R56P, E57N, E57D, E58G, E58Q, Y59F, Y59I, D60E, D60N, D60H, D60A, D60Q, D60Q, E61A, E61A, V62L, V62I, V62A, V62P, M63I, M63V, M63A, M63L, M63S, M63P, A64T, A64V, A64S, A64Y, D65S, D65N, A67R, H70N, H70D, P71S, R72K, R72L, R72E, T73K, T73A, T73G, T73D, S74A, L75F, P76R, P76G, T77V, L78M, K80R, K80R, T81A, T81W, T81V, I82V, I82V, A83G, S84G, S84P, S84T, S84G, T85S, T85A, L86I, E88D, E88P, E88A, Q89L, Q89H, Y90F, R91D, R91E, L92V, L92M, E94Q, E94A, Q95A, A96L, A96L, A96V, A96E, A96T, A96H, C97A, C97V, C97T, D98N, D99R, D99E, D99G, D99E, E100D, E100Q, V102I, M103L, L104I, L104V, L104I, L104V, L104V, G105A, S106H, S106N, S106R, L107S, L107Q, L107V, S111A, L115M, L115V, K118R, K118R, H119R, H119Y, I121A, I121V, V124A, V124I, T125S, A126G, A126V, A126G, Q127E, Q127H, V128A, V128L, V128I, V128A, S129T, S129Q, S129A, F131W, F131Q, F131H, G132N, G132Q, G132D, F133L, F133V, F133I, F133I, F133I, F133L, W134L, A136P, A136S, A137Q, A137K, A137F, A137L, A137R, A137H, A137M, A138S, A138D, S140A, S140P, S140P, S140P, T141V, T141I, T141V, H142L, H142S, P143-, P144A, P144K, P144K, P144L, G145P, G145R, A146T, A146F, A146T, A146L, A146S, A146L, N147T, N147P, N147S, L148I, L148V, A150E, A150T, A150G, F151S, F151E, F151D, F151T, F151W, F151W, V152L, V152L, P153T, P153S, Y154R, Y154D, Y154R, Y154V, Y154R, Y154L, P155E, V156I, V156P, V156D, V156Q, V156G, V156L, V156I, V156A, V156E, V156L, K157R, K157E, K157R, R158A, R158S, R158N, R158K, R158E, R158M, R158Q, L159E, L159D, L159G, L159R, L159A, L159K, L159T, L160F, L160I, L160A, L160V, L160Y, L161M, L161F, L161M, L161W, L161M, G162L, G162D, G162R, G162R, G162W, M163E, M163I, M163G, M163L, M163L, M163D, M163F, M163V, M163I, M163F, M163V, M163K, I164T, I164E, I164G, I164C, I164A, I164V, E165V, E165Y, E165D, E165A, E165I, R166K, R166L, R166E, R166V, R166Q, R166H, R166D, R166S, A167G, A167T, A167P, A167L, A167T, A167G, A167S, A167M, A167N, A167S, A167D, A167E, F168I, F168S, F168V, F168C, F168A, F168E, F168V, L169V, R171D, R171E, R171N, R171K, V172T, V172I, V172I, V172A, V172A, V172L, M173C, M173C, A174T, A174K, A174K, A174G, E176D, E176A, S179R, S179T, S179G, S179R, F180L, F180V, F180L, Q182S, Q182A, Q182R, Q182R, Q182G, Q182A, L186M, A187P, A187P, T189A, T189V, T189V, T189A, K190S, K190R, K190R, K190T, K190S, I192F, I193M, I193L, 1193F, S194G, S194G, Q195E, Q195R, I197L, I197V, S198H, S198A, S198H, D201Q, R202G, R202K, R202G, E209D, E209D, V214P, V214T, V214A, H216T, H216R, H216P, H216Q, H216S, H216S, A220S, A220S, Q221H, Q221N, Q221R, T222A, T222I, V223S, V223A, F230Y, S233A, S233A, S233G, S233T, G234S, G234D, L235D, L235E, L235G, L235F, Q236H, Q236E, Q236L, Q236H, Q236R, Q236E, Q236L, P237T, P237T, P237Q, P237E, P237A, P237Q, P237Q, L238V, L238P, L238T, L238M, L238I, L238R, L238V, A240T, A240P, A240D, E243D, D244A, D244E, D244E, N247A, N247A, N247S, N247S, N247Q, N247R, E248G, E248A, E248A, A249G, A249E, A249T, A249G, A249S, S250P, S250P, S250P, V253I, T259A, T259S, M260N, V261R, V261I, V261L, V261I, N262D, N262D, N262H, N262D, N262D, N262D, G263A, G263A, G263S, G263P, G263S, G263E, G263E, E264G, E264P, E264Q, E264T, E264D, E264A, Q265A, Q265T, Q265R, Q265R, Q265S, Y266F, T268R, T268A, T268S, A269T, V271A, V271I, V271L, V271A, V271A, Q272E, Q272E, L274C, K275R, K275R, L276S, L276R, L276K, L276A, L276R, I277T, I277L, I277A, I277L, I277M, N278G, N278G, V2821, F283L, T285A, S286G, S286R, S286K, S286R, S286R, Q287N, Q287D, Q287E, Q287G, S288P, S288T, S288A, S288E, S288G, V289D, V289A, V289T, V289P, D290E, D290L, D290V, P291D, P291A, P291H, P291Q, S292V, S292P, S292T, S292A, S292E, V293P, V293L, V293L, V293D, V293P, V293A, S294P, S294A, S294L, S295R, S295T, S295A, S295L, E296S, E296A, E296S, E296D, E296G, E296P, G297S, G297S, G297D, G297S, G297P, G297D, G297P, R298Q, R298Q, R298N, R298D, R298E, I299V, V301L, R303S, R303P, V305A, S308T, R309K, R309L, R309A, R309S, I310L, H313Q, H313R, S314T, S314A, S314A, S314A, S314C, S314C, A315S, A315T, L317I, V323I, V323I, T326A, T326V, L328Q, M330I, M330L, M330I, M330L, M330F, M330L, V334I, I337L, I337V, I337L, A338V, A338V, F341L, F346L, M352L, M352V, M352I, E353Q, C357V, C357V, L359I, L359V, V361L, V361I, V361L, V361I, V361L, F362S, F362D, F362L, F362R, F362R, F362L, F362K, F362L, F362S, F362P, F362D, P363G, P363A, P363R, P363S, P364S, P364R, P364K, A365V, S366T, S366Q, A367G, A367G, A367T, E368R, E368Q, S369R, S369P, S369A, L370I, A371S, A371T, A371T, T372A, T372R, D375Q, D375Q, D375R, D375E, K376R, K376T, K376R, K376R, I377L, I377V, N379D, N379K, N379K, N379G, N379D, S380D, S380D, S380D, S380E, Q381P, E382A, E382Q, E382A, V383I, V383I, R384S, R384A, R384T, A385E, A385E, S386N, S386A, S386N, S386K, S386R, S386A, E388T, E388A, E388A, E388D, E388Q, E388R, R389Q, R389K, R389E, Y390M, Y390T, Y390M, Y390L, Y390I, R391A, T392Q, T392A, T392K, T392K, T392P, T392S, T392K, T392K, L393R, L393R, L393Q, I394V, I394M, I394L, I394F, I394M, P395A, S396T, G397S, G397A, D398P, D398E, D398E, D398E, S399A, S399T, S399R, A400G, S401L, S401C, S401C, S401A, E402Q, A403S, A403R, A403T, A403K, L406S, L406E, L406D, L406N, L406R, Q407F, Q407A, Q407F, I408V, A410T, A410S, L411V, L411M, L411F, L411I, A412V, A412S, R413S, R413P, R413T, R413A, T414R, T414P, T414L, T414S, T414E, A415P, A415K, A415R, A415S, R416A, R416I, R416T, R416K, R416G, S417R, S417Y, S417A, S417K, R418Q, R418T, A419T, A419T, A419V and A420R in the glycosyltransferase amino acid sequence of SEQ ID NO: 1.

In some embodiments of the present invention, the method of making glycomonolipids by a single catalytic step by contacting a hydroxy fatty acid with a glycosyl donor in the presence of an enzyme may be done with an enzyme that may be a glycosyltransferase that comprises a deletion in at least one amino acid selected from E44, R45, C46, G47, F39, N49, F48, N49, F50, L51, P52, L53, G54, T55, R56, E57, E58, Y59, D60, E61, V62, P143, I164, D290, A415, R416, S417, R418, A419 and A420 in the glycosyltransferase amino acid sequence of SEQ ID NO: 1.

In some embodiments of the present invention, the method of making glycomonolipids by a single catalytic step by contacting a hydroxy fatty acid with a glycosyl donor in the presence of an enzyme may be done with an enzyme may be a glycosyltransferase that comprises an insertion to at least one amino acid selected from T392AV, I408VLS, S401CPA, E248AG, N247AG, R418APAG, E248GG, A420RVAA, R418VAAA, A37PT, P71GGTF, P71CGPGEGWW, P71GGRF, in the glycosyltransferase amino acid sequence of SEQ ID NO: 1. In these embodiments, “T392AV” represents insertion of alanine (A) and valine (V) subsequently following amino acid (T, or Thr) at position 392 in SEQ ID NO: 1.

In some embodiments of the present invention, the method of making glycomonolipids by a single catalytic step by contacting a hydroxy fatty acid with a glycosyl donor in the presence of an enzyme may be done with an enzyme may be a glycosidase comprising amino acids selected from E206, E387, H150, N205, R79, Q18, E432, W425, Y321, Y322, F222, W361, F441, V209, F359, Not D or E in 341, wherein said positions are numbered with reference to SEQ ID NO: 83.

In some embodiments of the present invention, the method of making glycomonolipids by a single catalytic step by contacting a hydroxy fatty acid with a glycosyl donor in the presence of an enzyme may be done with an enzyme may be a glycosidase comprising amino acids selected from E572, E841, D567, H862, N849, R858, D579, W864, W576, W679, W684, wherein said positions are numbered with reference to SEQ ID NO: 84.

In some embodiments, the present invention may be a glycosyltransferase variant that comprises a mutation in at least one amino acid selected from M1A, H2R, V3I, I4L, I4V, I5L, I5V, T6I, A7P, V8I, F17L, V18L, V18L, V18I, I20L, T23A, T23S, T23S, T23A, T23E, V25A, A26S, V31I, T32S, F33V, F33M, C34A, A35T, A35V, S36Y, S36N, S36F, A37P, A37EW, A37EF, A37PA, A37E, A37G, A37P, A38V, A38G, A38Y, A38R, F39L, F39A, A40K, A40R, A40P, A40E, A40S, P41E, P41S, P41A, L42S, L42A, I43A, I43V, I43L, E44D, E44G, R45L, R45G, R45E, C46S, C46A, C46P, C46R, G47A, F48M, F48L, N49S, N49A, N49E, N49T, N49G, N49G, N49K, N49E, N49R, N49S, F50V, F50L, L51I, L51V, L51V, L51V, P52A, L53V, L53R, L53I, G54T, T55E, T55S, T55P, R56A, R56A, R56A, R56P, E57N, E57D, E58G, E58Q, Y59F, Y59I, D60E, D60N, D60H, D60A, D60Q, D60Q, E61A, E61A, V62L, V62I, V62A, V62P, M63I, M63V, M63A, M63L, M63S, M63P, A64T, A64V, A64S, A64Y, D65S, D65N, A67R, H70N, H70D, P71S, R72K, R72L, R72E, T73K, T73A, T73G, T73D, S74A, L75F, P76R, P76G, T77V, L78M, K80R, K80R, T81A, T81W, T81V, I82V, I82V, A83G, S84G, S84P, S84T, S84G, T85S, T85A, L86I, E88D, E88P, E88A, Q89L, Q89H, Y90F, R91D, R91E, L92V, L92M, E94Q, E94A, Q95A, A96L, A96L, A96V, A96E, A96T, A96H, C97A, C97V, C97T, D98N, D99R, D99E, D99G, D99E, E100D, E100Q, V102I, M103L, L104I, L104V, L104I, L104V, L104V, G105A, S106H, S106N, S106R, L107S, L107Q, L107V, S111A, L115M, L115V, K118R, K118R, H119R, H119Y, I121A, I121V, V124A, V124I, T125S, A126G, A126V, A126G, Q127E, Q127H, V128A, V128L, V128I, V128A, S129T, S129Q, S129A, F131W, F131Q, F131H, G132N, G132Q, G132D, F133L, F133V, F133I, F133I, F133I, F133L, W134L, A136P, A136S, A137Q, A137K, A137F, A137L, A137R, A137H, A137M, A138S, A138D, S140A, S140P, S140P, S140P, T141V, T141I, T141V, H142L, H142S, P143-, P144A, P144K, P144K, P144L, G145P, G145R, A146T, A146F, A146T, A146L, A146S, A146L, N147T, N147P, N147S, L148I, L148V, A150E, A150T, A150G, F151S, F151E, F151D, F151T, F151W, F151W, V152L, V152L, P153T, P153S, Y154R, Y154D, Y154R, Y154V, Y154R, Y154L, P155E, V156I, V156P, V156D, V156Q, V156G, V156L, V156I, V156A, V156E, V156L, K157R, K157E, K157R, R158A, R158S, R158N, R158K, R158E, R158M, R158Q, L159E, L159D, L159G, L159R, L159A, L159K, L159T, L160F, L160I, L160A, L160V, L160Y, L161M, L161F, L161M, L161W, L161M, G162L, G162D, G162R, G162R, G162W, M163E, M163I, M163G, M163L, M163L, M163D, M163F, M163V, M163I, M163F, M163V, M163K, I164T, I164E, I164G, I164C, I164A, I164V, E165V, E165Y, E165D, E165A, E165I, R166K, R166L, R166E, R166V, R166Q, R166H, R166D, R166S, A167G, A167T, A167P, A167L, A167T, A167G, A167S, A167M, A167N, A167S, A167D, A167E, F168I, F168S, F168V, F168C, F168A, F168E, F168V, L169V, R171D, R171E, R171N, R171K, V172T, V172I, V172I, V172A, V172A, V172L, M173C, M173C, A174T, A174K, A174K, A174G, E176D, E176A, S179R, S179T, S179G, S179R, F180L, F180V, F180L, Q182S, Q182A, Q182R, Q182R, Q182G, Q182A, L186M, A187P, A187P, T189A, T189V, T189V, T189A, K190S, K190R, K190R, K190T, K190S, I192F, I193M, I193L, I193F, S194G, S194G, Q195E, Q195R, I197L, I197V, S198H, S198A, S198H, D201Q, R202G, R202K, R202G, E209D, E209D, V214P, V214T, V214A, H216T, H216R, H216P, H216Q, H216S, H216S, A220S, A220S, Q221H, Q221N, Q221R, T222A, T222I, V223S, V223A, F230Y, S233A, S233A, S233G, S233T, G234S, G234D, L235D, L235E, L235G, L235F, Q236H, Q236E, Q236L, Q236H, Q236R, Q236E, Q236L, P237T, P237T, P237Q, P237E, P237A, P237Q, P237Q, L238V, L238P, L238T, L238M, L238I, L238R, L238V, A240T, A240P, A240D, E243D, D244A, D244E, D244E, N247A, N247A, N247S, N247S, N247Q, N247R, E248G, E248A, E248A, A249G, A249E, A249T, A249G, A249S, S250P, S250P, S250P, V253I, T259A, T259S, M260N, V261R, V261I, V261L, V261I, N262D, N262D, N262H, N262D, N262D, N262D, G263A, G263A, G263S, G263P, G263S, G263E, G263E, E264G, E264P, E264Q, E264T, E264D, E264A, Q265A, Q265T, Q265R, Q265R, Q265S, Y266F, T268R, T268A, T268S, A269T, V271A, V271I, V271L, V271A, V271A, Q272E, Q272E, L274C, K275R, K275R, L276S, L276R, L276K, L276A, L276R, I277T, I277L, I277A, I277L, 1277M, N278G, N278G, V282I, F283L, T285A, S286G, S286R, S286K, S286R, S286R, Q287N, Q287D, Q287E, Q287G, S288P, S288T, S288A, S288E, S288G, V289D, V289A, V289T, V289P, D290E, D290L, D290V, P291D, P291A, P291H, P291Q, S292V, S292P, S292T, S292A, S292E, V293P, V293L, V293L, V293D, V293P, V293A, S294P, S294A, S294L, S295R, S295T, S295A, S295L, E296S, E296A, E296S, E296D, E296G, E296P, G297S, G297S, G297D, G297S, G297P, G297D, G297P, R298Q, R298Q, R298N, R298D, R298E, I299V, V301L, R303S, R303P, V305A, S308T, R309K, R309L, R309A, R309S, I310L, H313Q, H313R, S314T, S314A, S314A, S314A, S314C, S314C, A315S, A315T, L317I, V323I, V323I, T326A, T326V, L328Q, M330I, M330L, M330I, M330L, M330F, M330L, V334I, I337L, I337V, I337L, A338V, A338V, F341L, F346L, M352L, M352V, M352I, E353Q, C357V, C357V, L359I, L359V, V361L, V361I, V361L, V361I, V361L, F362S, F362D, F362L, F362R, F362R, F362L, F362K, F362L, F362S, F362P, F362D, P363G, P363A, P363R, P363S, P364S, P364R, P364K, A365V, S366T, S366Q, A367G, A367G, A367T, E368R, E368Q, S369R, S369P, S369A, L370I, A371S, A371T, A371T, T372A, T372R, D375Q, D375Q, D375R, D375E, K376R, K376T, K376R, K376R, I377L, I377V, N379D, N379K, N379K, N379G, N379D, S380D, S380D, S380D, S380E, Q381P, E382A, E382Q, E382A, V383I, V383I, R384S, R384A, R384T, A385E, A385E, S386N, S386A, S386N, S386K, S386R, S386A, E388T, E388A, E388A, E388D, E388Q, E388R, R389Q, R389K, R389E, Y390M, Y390T, Y390M, Y390L, Y390I, R391A, T392Q, T392A, T392K, T392K, T392P, T392S, T392K, T392K, L393R, L393R, L393Q, I394V, I394M, I394L, I394F, I394M, P395A, S396T, G397S, G397A, D398P, D398E, D398E, D398E, S399A, S399T, S399R, A400G, S401L, S401C, S401C, S401A, E402Q, A403S, A403R, A403T, A403K, L406S, L406E, L406D, L406N, L406R, Q407F, Q407A, Q407F, I408V, A410T, A410S, L411V, L411M, L411F, L411I, A412V, A412S, R413S, R413P, R413T, R413A, T414R, T414P, T414L, T414S, T414E, A415P, A415K, A415R, A415S, R416A, R416I, R416T, R416K, R416G, S417R, S417Y, S417A, S417K, R418Q, R418T, A419T, A419T, A419V and A420R in the glycosyltransferase amino acid sequence of SEQ ID NO: 1.

In some embodiments, the present invention may be a glycosyltransferase variant that comprises a deletion in at least one amino acid selected from E44, R45, C46, G47, F39, N49, F48, N49, F50, L51, P52, L53, G54, T55, R56, E57, E58, Y59, D60, E61, V62, P143, I164, D290, A415, R416, S417, R418, A419 and A420 in the glycosyltransferase amino acid sequence of SEQ ID NO: 1.

In some embodiments, the present invention may be a glycosyltransferase variant that comprises an insertion to at least one amino acid selected from T392AV, I408VLS, S401CPA, E248AG, N247AG, R418APAG, E248GG, A420RVAA, R418VAAA, A37PT, P71GGTF, P71CGPGEGWW, and P71GGRF in the glycosyltransferase amino acid sequence of SEQ ID NO: 1.

As used herein, “amplify,” “amplified,” or “amplification” refers to any process or protocol for copying a polynucleotide sequence into a larger number of polynucleotide molecules, e.g., by reverse transcription, polymerase chain reaction, and ligase chain reaction.

As used herein, an “antisense sequence” refers to a sequence that specifically hybridizes with a second polynucleotide sequence.

As used herein, “cDNA” refers to a DNA that is complementary or identical to an mRNA, in either single stranded or double stranded form. As used herein, the carbons in fatty acids are numbered with the first carbon as part of the carboxylic acid group, and the second carbon adjacent to the first. The numbers continue so that the highest number carbon is farthest from the carboxylic acid group.

As used herein, “complementary” refers to a polynucleotide that can base pair with a second polynucleotide. For example, a polynucleotide having the sequence 5′-GTCCGA-3′ is complementary to a polynucleotide with the sequence 5′-TCGGAC-3′.

As used herein, a “conservative substitution” refers to the substitution in a polypeptide of an amino acid with a functionally similar amino acid.

As used herein, “encoding” refers to the inherent property of nucleotides to serve as templates for synthesis of other polymers and macromolecules. Unless otherwise specified, a “nucleotide sequence encoding an amino acid sequence” includes all nucleotide sequences that are degenerate versions of each other and that encode the same amino acid sequence.

As used herein, “endogenous” refers to polynucleotides, polypeptides, or other compounds that are expressed naturally or originate within an organism or cell. That is, endogenous polynucleotides, polypeptides, or other compounds are not exogenous.

As used herein, “expression vector” refers to a vector comprising a recombinant polynucleotide comprising expression control sequences operatively linked to a nucleotide sequence to be expressed. For example, suitable expression vectors can be an autonomously replicating or integrated into the chromosome.

As used herein, “exogenous” refers to any polynucleotide or polypeptide that is not naturally expressed in the particular cell or organism where expression is desired. Exogenous polynucleotides, polypeptides, or other compounds are not endogenous.

As used herein, “hybridization” includes any process by which a strand of a nucleic acid joins with a complementary nucleic acid strand through base-pairing. Thus, the term refers to the ability of the complement of the target sequence to bind to a test sequence, or vice-versa.

As used herein, “hybridization conditions” are typically classified by degree of “stringency” of the conditions under which hybridization is measured. The degree of stringency can be based, for example, on the melting temperature (Tm) of the nucleic acid binding complex or probe. For example, “maximum stringency” typically occurs at about Tm-5° C. (5° C. below the Tm of the probe); “high stringency” at about 5-10° C. below the Tm; “intermediate stringency” at about 10-20° below the Tm of the probe; and “low stringency” at about 20-25° C. below the Tm. Alternatively, or in addition, hybridization conditions can be based upon the salt or ionic strength conditions of hybridization and/or one or more stringency washes. For example, 6×SSC (saline sodium citrate)=very low stringency; 3×SSC=low to medium stringency; 1×SSC=medium stringency; and 0.5×SSC=high stringency. Functionally, maximum stringency conditions may be used to identify nucleic acid sequences having strict identity or near-strict identity with the hybridization probe; while high stringency conditions are used to identify nucleic acid sequences having about 80% or more sequence identity with the probe.

As used herein, “identical” or percent “identity,” in the context of two or more polynucleotide or polypeptide sequences, refers to two or more sequences that are the same or have a specified percentage of nucleotides or amino acid residues that are the same, when compared and aligned for maximum correspondence, as measured using sequence comparison algorithms or by visual inspection.

As used herein, “long-chain fatty acids” refers to fatty acids with aliphatic tails longer than 14 carbons.

As used herein, “medium-chain fatty acids” refers to fatty acids with aliphatic tails between 6 and 14 carbons. In certain embodiments, the medium-chain fatty acids can have from 11 to 13 carbons.

As used herein, “naturally-occurring” or “wildtype” refers to an object that can be found in nature. For example, a polypeptide or polynucleotide sequence that is present in an organism (including viruses) that can be isolated from a source in nature, and which has not been intentionally modified by man in the laboratory is naturally-occurring or wildtype.

As used herein, “operably linked”, when describing the relationship between two DNA regions or two polypeptide regions, simply means that they are functionally related to each other. For example, a promoter is operably linked to a coding sequence if it controls the transcription of the sequence; a ribosome binding site is operably linked to a coding sequence if it is positioned to permit translation; and a sequence is operably linked to a peptide if it functions as a signal sequence, such as by participating in the secretion of the mature form of the protein.

As used herein, “polynucleotide” refers to a polymer composed of nucleotides. The polynucleotide may be in the form of a separate fragment or as a component of a larger nucleotide sequence construct, which has been derived from a nucleotide sequence isolated at least once in a quantity or concentration enabling identification, manipulation, and recovery of the sequence and its component nucleotide sequences by standard molecular biology methods, for example, using a cloning vector. When a nucleotide sequence is represented by a DNA sequence (i.e., A, T, G, C), this also includes an RNA sequence (i.e., A, U, G, C) in which “U” replaces “T”

As used herein, “polypeptide” refers to a polymer composed of amino acid residues which may or may not contain modifications such as phosphates and formyl groups.

As used herein, “recombinant expression vector” refers to a DNA construct used to express a polynucleotide that encodes a desired polypeptide and which can include, for example, a transcriptional subunit comprising an assembly of genetic elements having a regulatory role in gene expression, for example, promoters and enhancers, a structural or coding sequence which is transcribed into mRNA and translated into protein, and appropriate transcription and translation initiation and termination sequences. Recombinant expression vectors can be constructed in any suitable manner. The nature of the vector is not critical, and any vector may be used, including plasmid, virus, bacteriophage, and transposon. Possible vectors for use in the present invention include, but are not limited to, chromosomal, nonchromosomal and synthetic DNA sequences, e.g., bacterial plasmids; phage DNA; yeast plasmids; vectors derived from combinations of plasmids and phage DNA, DNA from viruses such as vaccinia, adenovirus, fowl pox, baculovirus, SV40, and pseudorabies.

As used herein, “primer” refers to a polynucleotide that is capable of specifically hybridizing to a designated polynucleotide template and providing a point of initiation for synthesis of a complementary polynucleotide when the polynucleotide primer is placed under conditions in which synthesis is induced.

As used herein, “recombinant polynucleotide” refers to a polynucleotide having sequences that are not naturally joined together. A recombinant polynucleotide may be included in a suitable vector, and the vector can be used to transform a suitable host cell. A host cell that comprises the recombinant polynucleotide is referred to as a “recombinant host cell.” The gene is then expressed in the recombinant host cell to produce, e.g., a “recombinant polypeptide.”

As used herein, “specific hybridization” refers to the binding, duplexing, or hybridizing of a polynucleotide preferentially to a particular nucleotide sequence under stringent conditions.

As used herein, “stringent conditions” refers to conditions under which a probe will hybridize preferentially to its target subsequence, and to a lesser extent to, or not at all to, other sequences.

As used herein, “short-chain fatty acids” refers to fatty acids having aliphatic tails with fewer than 6 carbons.

As used herein, “substantially homologous” or “substantially identical” in the context of two nucleic acids or polypeptides, generally refers to two or more sequences or subsequences that have at least 40%, 60%, 80%, 90%, 95%, 96%, 97%, 98% or 99% nucleotide or amino acid residue identity, when compared and aligned for maximum correspondence, as measured using sequence comparison algorithms or by visual inspection. The substantial identity can exist over any suitable region of the sequences, such as, for example, a region that is at least about 50 residues in length, a region that is at least about 100 residues, or a region that is at least about 150 residues. In certain embodiments, the sequences are substantially identical over the entire length of either or both comparison biopolymers.

As used herein, “NMP” is defined as a nucleotide base monophosphate and “NDP” is defined as a nucleotide base diphosphate, where N is any of the DNA or RNA nucleotide bases i.e. A (Adenine) or T (Thymine) or C (Cytosine) or G (Guanine) or U (Uracil).

The compositions resulting from the inventive method may comprise rhamnolipids in which at least about 25% by weight of all the rhamnolipids are-monolipids. In some embodiments, the weight % of all rhamnolipids in a composition that is-monolipids may be at least about 15%, at least about 20%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or at least about 100%. In some embodiments, the weight % of all rhamnolipids in a composition that are-mono-lipids may be from about 25%, about 30%, about 35%, about 40%, about 45%, about 50%, about 55%, about 60%, about 65%, about 70%, about 75%, about 80%, about 85%, about 90%, about 95%, to about 40%, about 45%, about 50%, about 55%, about 60%, about 65%, about 70%, about 75%, about 80%, about 85%, about 90%, about 95%, about 96%, about 97%, about 98%, about 99%, or about 100%, with any combination of these percentages herein.

In some embodiments, a composition resulting from the inventive method may comprise rhamnolipids in which monorhamnomonolipids comprise at least about 20%, about 30%, about 40%, about 50%, about 60%, about 70%, about 80%, about 90%, about 95%, about 99%, or about 100% by weight of all the rhamnolipids, or in some embodiments from about 20% to about 100%, or about 25% to about 95%, or any combination therebetween, by weight of all rhamnolipids.

### EXAMPLES

### Example A

### Preparation of Escherichia coli Expression System Containing Rhamnosyltransferase Genes

The rhamnosyltransferases (SEQ ID NOs: 1-12) glycosylate lipids into corresponding rhamnolipids. Rhamnosyltransferase from Pseudomonas mediterranea (SEQ ID NO: 1) was further engineered in silico to design mutants (SEQ ID NOs: 63-70) with improved activity of the glycosylation of 3-hydroxy fatty acids into corresponding rhamnomonolipids, Codon optimized genes (SEQ ID NOs: 18-29 and mutant sequences, SEQ ID NOs: 71-78) encoding for uncultured bacterium clones of rhamnosyltransferase are designed and synthesized. After gene synthesis, the complete synthetic gene sequences are independently subcloned into a pCOLADuet-1 or pET30a vector (GenScript, Piscataway, NJ, U.S.A.) for heterologous expression. Escherichia coli BL21 Star (DE3) cells (Invitrogen, Thermo Fisher Scientific, Waltham, MA, USA) are transformed with the recombinant plasmid.

### Example B

### Preparation of FLAG Tagged Rhamnosyltransferases

A codon optimized gene encoding for rhmanosyltransferases SEQ ID NOs: 18 and 21 were further designed and synthesized to include FLAG tag amino acid sequence (DYKDDDDK) to SEQ ID NOs: 1 and 4. The resulting FLAG tagged proteins (SEQ ID NOs: 13 and 16) are expressed in Luria broth (Thermo Fisher Scientific, Waltham, MA, USA) medium containing kanamycin and induced with 1 mM IPTG at 30° C. for 12 hours at 225 rpm. Cells are harvested by centrifugation (3000 rpm for 30 minutes), lysed and protein in the supernatant is purified using immobilized FLAG-tag specific monoclonal antibody for FLAG tagged proteins and eluted with FLAG-peptide (GenScript, Piscataway, NJ, U.S.A). The proteins were stored at −80° C. in buffer containing 50 mM Tris-HCl, pH 8.0, 500 mM NaCl, and 10% glycerol.

### Inventive Example C

### Preparation of Rhamnomonolipids by Rhamnosyltranferase in a Recombinant Host Cell

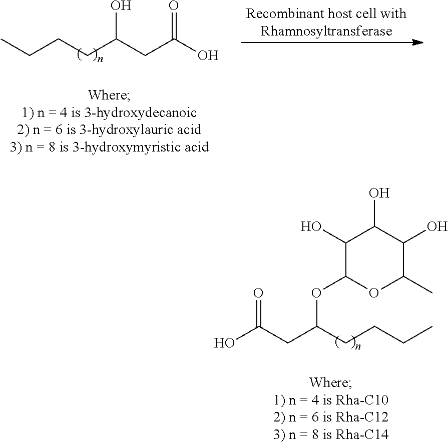

a single colony of the transformed Escherichia coli BL21 Star (DE3) (Invitrogen, Thermo Fisher Scientific, Waltham, MA, USA) cells for SEQ ID NOs: 18-29 from Example A is inoculated into Luria broth (Thermo Fisher Scientific, Waltham, MA, USA) medium containing kanamycin (Teknova, Hollister, California, USA). Magic medium (Thermo Fisher Scientific, Waltham, MA, USA) is inoculated with either 3-hydroxydecanoic acid (Sigma-Aldrich) or 3-hydoxylauric acid (Sigma-Aldrich, St. Louis, MO, USA) or 3-hydroxymyristic acid (TCI America, Portland, OR, USA) at final concentration of 1 mM. The mixture is then inoculated with culture grown from the Luria broth at a 1:100 dilution, and incubated at 37° C. for 24 h at 225 rpm. Supernatant is separated from the cell pellet by centrifugation and rhamnomonolipid produced is analyzed by Test Method 1 and results shown in Table 1.

TABLE 1
Quantification of rhamnomonolipids produced
from one step transformation by rhamnosyltransferases
in Escherichia coli

| Rhamnomonolipid | Rhamnosyl- | Total Quantified Rhamnolipid |
|---|---|---|
| formed | transferase | (Area Under Curve) |
| Rha-C10 | SEQ ID NO: 1 | 8204382 |
| | SEQ ID NO: 2 | 873518 |
| | SEQ ID NO: 4 | 3803186 |
| | SEQ ID NO: 7 | 185418 |
| | SEQ ID NO: 17 | 0 |
| Rha-C12 | SEQ ID NO: 1 | 37410942 |
| | SEQ ID NO: 2 | 803439 |
| | SEQ ID NO: 4 | 28436386 |
| | SEQ ID NO: 6 | 513267 |
| | SEQ ID NO: 17 | 0 |
| Rha-C14 | SEQ ID NO: 1 | 5431332 |
| | SEQ ID NO: 4 | 12943919 |
| | SEQ ID NO: 6 | 20883 |
| | SEQ ID NO: 17 | 0 |

As shown in table 1, rhamnomonolipids were produced when the rhamnosyltransferase genes were expressed for SEQ ID NOs: 1, 2, 4, 6 or7 in comparison to the reference out-sequence of canonical rhamnosyltransferase from Pseudomonas fluorescens (SEQ ID NO: 17).

### Example D

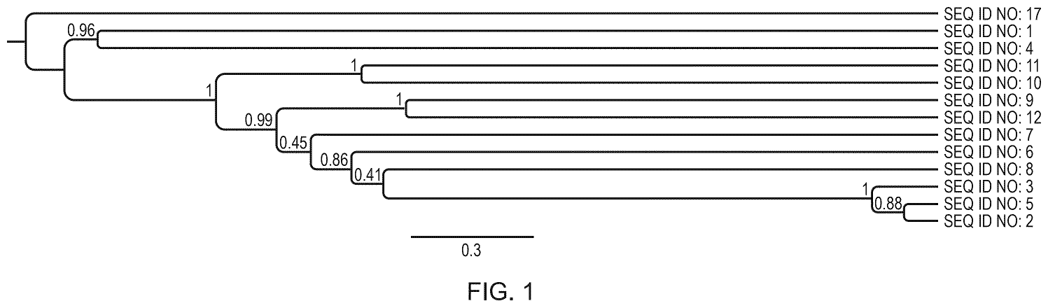

As shown in FIG. 1, the sequences were aligned via MUltiple Sequence Comparison by Log-Expectation (MUSCLE) version 5.1 (Edgar, RC (2021), MUSCLE v5 enables improved estimates of phylogenetic tree confidence by ensemble bootstrapping, bioRxiv 2021 Jun. 20) with the Super5 algorithm and their phylogenetic inferred via FastTree version 2.1.11 with a optimization for Gamma20 likelihood over 20 category sites, for 12 sequences in comparison to our reference out-sequences of canonical RhlB (WP_003092007.1 from Pseudomonas fluorescens—SEQ ID NO: 17). Numbers at the branching points, bootstrap values, indicate the likelihood of the branching (scale 0-1.0, where 1.0 designates 100% likelihood) suggesting closer likeliness towards that branch or relationship between those two enzyme members of that associated branch. The scale bar at the bottom is indicative of the numeric differences of amino acids per site. Table 2 shows the rhamnosyltransferase species and corresponding sequence ID.

TABLE 2

| Rhamnosyltransferase Species Identifier | Sequence ID |
|---|---|
| Pseudomonas mediterranea | SEQ ID NO: 1 |
| Lonsdalea britannica | SEQ ID NO: 2 |
| Lonsdalea iberica | SEQ ID NO: 3 |
| Pseudomonas corrugata | SEQ ID NO: 4 |
| Lonsdalea quercina | SEQ ID NO: 5 |
| * Phycisphaeraceae sp. | SEQ ID NO: 6 |
| * Nevskia sp. | SEQ ID NO: 7 |
| Streptococcus dysgalactiae subspp. Equisimilis | SEQ ID NO: 8 |
| * Granulicella sp. | SEQ ID NO: 9 |
| Paraburkholderia asphalathi | SEQ ID NO: 10 |
| Robbsia andropogonis | SEQ ID NO: 11 |
| Granulicella arctica | SEQ ID NO: 12 |
| Pseudomonas fluorescens | SEQ ID NO: 17 |

* = Indicates closest BLASTp hits on NCBI

### Example E

### Improved Activity of Mutant Rrhamnosyltransferases (SEQ ID NOs: 63-70) to Produce Rhamnomonolipids, Compared to Wwildtype.

Mutant rhamnosyltransferases were tested in a recombinant cell. Briefly, a single colony of the transformed Escherichia coli BL21 Star (DE3) cells (Invitrogen) for SEQ ID NOs: 71-78 from Example A, or competent Pseudomonas putida as described in Test Method 3 transformed with SEQ ID NO: 81 or SEQ ID NO: 82, were inoculated into Luria broth (Thermo Fisher Scientific, Waltham, MA, USA) medium containing 50 μg/mL kanamycin (Teknova, Hollister, CA, USA). Magic medium (Invitrogen) (for expression in Escherichia coli) or Luria broth medium (for expression in Pseudomonas putida) were mixed with either 3-hydroxydecanoic acid (Sigma Aldrich, St. Louis, MO, USA) or 3-hydoxylauric acid (Sigma Aldrich, St. Louis, MO, USA) or 3-hydroxymyristic acid (TCI America. Portland, OR, USA) at final concentration of 1 mM. These mixtures were then inoculated with the culture grown from the Luria broth above, containing SEQ ID NOs: 71-78 (in Escherichia coli), or SEQ ID NO: 81 or SEQ ID NO: 82 (in Pseudomonas putida) at a 1:100 dilution, and incubated at 30° C. for 20 hours (Pseudomonas putida) or 37° C. for 24 hours (in Escherichia coli) at 225 rpm. Supernatant is separated from the cell pellet by centrifugation and rhamnomonolipid production was analyzed by Test Method 1. Results are shown for quantification of Rha-C10 in Table 3, Rha-C12 in Table 4, and Rha-C14 in Table 5 for Escherichia coli; and in Table 6 of Rha-C10 in Pseudomonas putida. Activity Improvement is calculated by comparing the quantity of mono-rhamno-mono-lipid produced by the mutants, divided by the quantity of mono-rhamno-mono-lipid produced by wildtype (SEQ ID NO: 1). Rhamnomonolipid production was significantly improved for the mutant enzymes (SEQ ID NOs: 63-70) compared to the wildtype SEQ ID NO: 1. When the Pseudomonas putida host is used in particular, the Activity Improvement is surprisingly increased by greater than 80-fold (Table 6).

TABLE 3
Quantification of Rha-C10 produced from one step transformation
by mutant rhamnosyltransferases and Activity Improvement compared
to control wildtype, in Escherichia coli.

| Rhamnosyltransferase | | Activity |
|---|---|---|
| ID | SEQ ID NO | Improvement |
| P. mediterranea - | 1 | 1.0 |
| wild type control | | |
| mutant 1 | 63 | 1.2 |
| mutant 2 | 64 | 4.46 |
| mutant 3 | 65 | 3.00 |
| mutant 5 | 67 | 1.1 |
| mutant 6 | 68 | 2.45 |
| mutant 7 | 69 | 1.1 |
| mutant 8 | 70 | 1.4 |

TABLE 4
Quantification of Rha-C12 produced from one step transformation
by mutant rhamnosyltransferases and Activity Improvement compared
to control wildtype, in Escherichia coli.

| Rhamnosyltransferase | | |
|---|---|---|
| ID | SEQ ID NO | Activity Improvement |
| P. mediterranea - | 1 | 1.0 |
| wild type control | | |
| mutant 1 | 63 | 1.5 |
| mutant 2 | 64 | 1.6 |
| mutant 3 | 65 | 1.1 |
| mutant 4 | 66 | 1.2 |
| mutant 6 | 68 | 1.1 |

TABLE 5
Quantification of Rha-C14 produced from one step transformation
by mutant rhamnosyltransferases and Activity Improvement compared
to control wildtype, in Escherichia coli.

| Rhamnosyltransferase | | Activity |
|---|---|---|
| ID | SEQ ID NO | Improvement |
| P. mediterranea - | 1 | 1.0 |
| wild type control | | |
| mutant 1 | 63 | 1.4 |
| mutant 2 | 64 | 2.32 |
| mutant 3 | 65 | 2.25 |
| mutant 6 | 68 | 1.9 |

TABLE 6
Quantification of Rha-C10 produced from one step transformation
by mutant rhamnosyltransferases and Activity Improvement compared
to control wildtype, in Pseudomonas putida.

| Rhamnosyltransferase | | Activity |
|---|---|---|
| ID | SEQ ID NO | Improvement |
| P. mediterranea - | 1 | 1.0 |
| wild type control | | |
| Empty Vector Control | — | — |
| Medium Control | — | — |
| Mutant 1 | 63 | 133.51 |
| Mutant 2 | 64 | 80 |

### Example F

### Preparation of Rhamnomonolipids by Rhamnosyltranferase in a Cell Free System

A purified rhamnosyltransferase (SEQ ID NO: 1 or SEQ ID NO: 4) is mixed with 0.1 M Sodium phosphate buffer at pH 7.4 (Sigma Aldrich); 0.5 mg/mL TDP-Rhamnose (BOC Sciences) and 1.0 mg/mL of 3-hydroxy decanoic acid (Sigma Aldrich) dissolved in DMSO (Thermo Fischer) to give a final concentration of 5% to a reaction volume of 500 μL. The mixture was incubated at 25° C. temperature under 300 rpm agitation for 48 hours reaction. Resulting Rha-C10 was analyzed using Test Method 1. Table 7 shows a summary of the results of SEQ ID NO: 1 and SEQ ID NO: 4 of quantified Rha-C10 formed.

TABLE 7
Quantification of Rha-C10 produced from one step transformation
by rhamnosyltransferases in a cell free system

| | | Total | Ratio of |
|---|---|---|---|
| | | Quantified | Rhamnolipid |
| | | Rhamnolipid | formed in |
| Rhamnomonolipid | | (Area Under | enzyme vs. |
| formed | Rhamnosyltransferase | Curve) | no enzyme |
| Rha-C10 | SEQ ID NO: 1 | 11152 | 1.2 |
| | SEQ ID NO: 4 | 17056 | 1.9 |
| | No Enzyme Control | 9184 | 1 |

### Example G

### Purification of Glycosidases

SEQ ID NO: 30 and SEQ ID NO: 80 were expressed according to Test Method 4. All purification steps were carried out on ice or at 4° C., unless otherwise specified using a cobalt affinity resin that recognizes 6×histidine tag. HisPur™ Cobalt Resin (Thermo Fisher) (5 mL) was transferred to a 50 mL falcon tube and centrifuged for 2 minutes at 800×g at 4° C., the supernatant was discarded. Following equilibration with 10 mL of wash buffer (50 mM sodium phosphate, 300 mM NaCl and 10 mM imidazole pH 7.4), 10 mL of the cell free extracts mixed with an equal volume of wash buffer were added to the cobalt resin and incubated at 150 rpm for 60 min at 16° C. The supernatant was discarded by centrifugation for 2 minutes at 800×g. The resin was washed five times with 10 mL of wash buffer and the adsorbed protein was eluted using 5 mL of elution buffer (50 mM sodium phosphate, 300 mM NaCl and 100 mM imidazole pH 7.4). The elution step was repeated two times, and the fractions were pooled together, concentrated to ˜1 mL and diluted with 10 mL in 0.1 M Phosphate buffer, pH 6 two times with Amicon ultra-0.5 centrifugal filters with a 30 kDa Molecular weight cut off membrane (Millipore) to reduce imidazole to a minimum. The purified enzymes were concentrated to 1 mL in a final step and stored at 4° C. SDS-PAGE analysis was employed to determine the purity and concentration of the purified enzymes.

### Example H

### Production of Glycomonolipids by Glycosidases in a Cell Free System

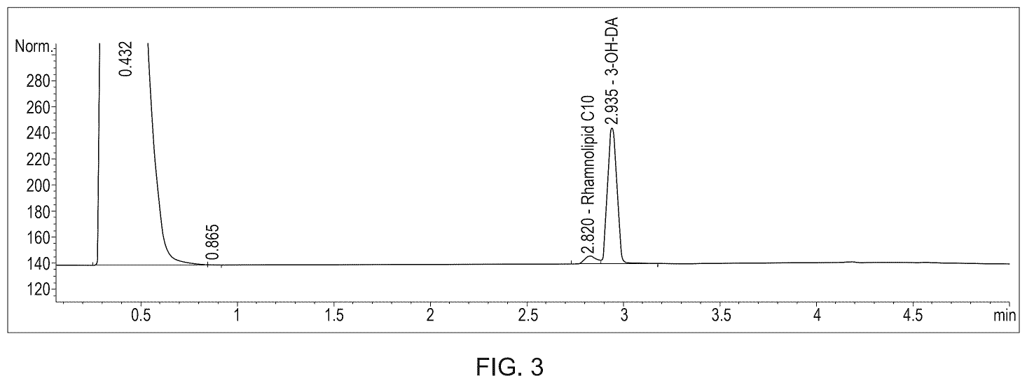

A purified glycosidase (SEQ ID NO: 30 or SEQ ID NO: 80) is mixed with 0.1 M Sodium phosphate buffer at pH 7 or 75% Glycerol; 1.5 mg/mL sugar and 3 mg/mL of 3-hydroxy decanoic acid (Sigma Aldrich, St. Louis, MO, USA) to a final volume of 300 μL. The mixture was incubated at 15° C. temperature under 1300 rpm agitation for 72-96 hours reaction. Reaction product was then analyzed via achiral HPLC with ELSD according to Test Method 4 to determine quantitative glycomonolipid formed. FIG. 3 is a chromatogram of formation of Rha-C10 by SEQ ID NO: 30. After 72 hours of reaction of rhamnose and 3-OH-decanoic acid in 75% glycerol at 15° C. Rha-C10 formed was traced at 2.820 minutes retention time. Table 8 is a summary of the results of the glycosidases ability to glycosylate 3-hydroxydecanoic acid to forma Rha-C10 in phosphate buffer at pH 7.0 or in 75% glycerol.

TABLE 8
Quantification of Rha-C10 produced from one step transformation
by glycosidases in a cell free system

| Solvent Used | Glycosidase | % Rha-C10 Formed |
|---|---|---|
| Phosphate Buffer pH 7 | SEQ ID NO: 30 | 0.3% |
| Phosphate Buffer pH 7 | SEQ ID NO: 80 | 0.3% |
| 75% glycerol | SEQ ID NO: 30 | 11.4% |
| 75% glycerol | SEQ ID NO: 80 | 0 |

### Example I

### Production of Mono-Rhamno-Mono-lLpid by an Immobilized Glycosidase

Recombinant glycosidase (SEQ ID NO: 30) in 100 mM phosphate buffer pH 7.4 is applied to affinity resin (e.g., Nickel affinity resin; Cytiva HisTrap HP). The mixture is allowed to incubate for 30 minutes at 4° C. while shaking to immobilize the enzyme. The un-bound enzyme is eluted followed by treatment of immobilized enzyme with 3-hydroxy decanoic acid (final concentration of 4 mg/mL) and alpha-rhamnose (final concentration of 2.2 mg/mL) in 5 mL 75% glycerol/25% water. The mixture is allowed to react for 3 days at 25° C. with shaking at 225 rpm. The soluble mixture is collected and rhamno-mono-lipid formation is analyzed by Test Method 1. Table 9 shows the quantified Rha-C10 results.

TABLE 9
Quantification of Rha-C10 produced from one step
transformation by immobilized glycosidase

| | | Total Quantified |
|---|---|---|
| Rhamnomonolipid | | Rhamnolipid (Area |
| formed | Glycosidase | Under Curve) |
| Rha-C10 | SEQ ID NO: 30 | 2624 |
| | No Enzyme Control | 0 |

### TEST METHODS

### 1. Analysis by LC-MS/MS

In Liquid Chromatography-Mass Spectrometry/Mass Spectrometry (LC-MS/MS), the “area under the curve” refers to the total area beneath a chromatographic peak on a mass spectrum, which directly corresponds to the quantity of a specific analyte present in a sample, used for quantitative analysis by calculating the area under the peak to determine its concentration.

Chromatography consisted of an ACQUITY BEH C18 Vanguard precolumn (130 Å, 1.7 μm, 2.1 mm×5 mm, Waters, Milford, MA, USA) followed by an ACQUITY UPLC BEH C18 Column (130Å, 1.7 μm, 2.1 mm'100 mm, Waters, Milford, MA, USA). The LC gradient was carried out on ExionLCTM AD UPLC system (AB Sciex, Framingham, MA, USA) from 5 to 95% B (Acetonitrile) over 10.5 min, for a total run time of 15 min. Mobile Phase A was 4 mM Ammonium Acetate in Water, and the flow rate was set to 0.4 mL/min. Samples were injected onto the instrument using PAL HTS-xt autosampler (CTC Analytics, Zwingen, Switzerland).

Eluted Rhamnolipids from the UPLC column were analyzed by tandem mass spectrometry on a Triple Quad 6500+ system (AB Sciex, Framingham, MA, USA). The instrument was operated in multiple reaction monitoring (MRM) and negative electrospray ionization (ESI) mode.

All data processing was performed using the Skyline Software, Version 23.1.0.268 (MacCoss Lab, University of Washington, Seattle, WA, USA). The transition results were exported to CSV file for further analysis. For relative quantitation purpose, carbon-13 stable isotope labeled Rhamnolipids was prepared by fermentation using D-glucose (U-13C6) as the sole carbon source. The list of Rhamnolipids and corresponding transitions is included.

### 2. Fermentation of Microbial Cultures

Suitable conditions consisting of a fermentation broth having a concentration of rhamnolipids were comprised of: a bacterial seed culture, preferably a strain of Pseudomonas putida bacteria; preparing a fermentation medium in a suitable fermentation vessel equipped to agitate and aerate a fermentation broth, the fermentation medium comprising a carbon source selected from the group selected from fatty acid distillate, used soybean oil, soybean oil soapstock, orange peels, distillery waste, wheat straw, sweet water, or sugarcane begasse oil, soybean oil, rapeseed oil, cocoa butter, olive oil, rice bran oil, palm oil, animal fat, glycerol, fatty acids, used cooking oil, waste oil, waste grease, glucose, fructose, sucrose, lactose, maltose, corn syrup, corn molasses, soy molasses, carbohydrates, materials containing carbohydrates, glycerides, fatty acids, glycerol, and combinations thereof, a nitrogen source and a non-nitrogen source and combinations therein. Aion and aeration of the fermentation broth was performed at a temperature of from 15° C. to 40° C.; more preferably 30° C.

### 3. Preparation of Pseudomonas putida Competent Cells

A 5 mL Pseudomonas putida (ATCC 47054) culture was grown overnight in Luria Broth (Thermo Fisher Scientific, Waltham, MA, USA). The culture was washed in sucrose three times by centrifuging, decanting, and resuspending in 1 mL of 300 mM sucrose. After the final wash, the cell pellet was resuspended in 50 μL of 300 mM sucrose and transferred to a BioRad Gene Pulser 0.1 cm electroporation cuvette (Bio-Rad Laboratories, Hercules, CA, USA), electroporated at 2.5 kV, 200 ohm resistance, 25 uF capacitance, and incubated in super optimal broth with catabolite repression (SOC) medium (Thermo Fisher Scientific, Waltham, MA, USA) at 30° C., 225 rpm for 1 hour, then plated on LB kanamycin-50 plates (Teknova, Hollister, CA, USA) overnight.

### 4. Preparation of Glycosidase Lysates

Glycosidase enzymes were expressed according to procedures common in the art. Briefly, a single colony of transformed competent Escherichia coli cells is inoculated into Luria Broth (LB) or Terrific Broth (TB; Research Products International, Mt. Prospect, IL, USA) medium containing appropriate antibiotic. LB or TB medium is inoculated with culture grown from the LB or TB at a 1:100 dilution and incubated at 25° C. for until appropriate optical density at 600 nm, followed by induction with 1 mM IPTG. Cells are harvested by centrifugation and the pellets are lysed with procedures common in the art. The supernatant is collected, utilized for enzymatic assays without further purification unless stated otherwise.

### 5. Achiral HPLC Method for Analysis of Glycosidase Reaction

Method was developed with parameters as shown in Table 8. An ELSD (Evaporative Light for Scattering Detector) was used to monitor glycosidase activity. The evaporator and nebuliser temperature was 40° C.

TABLE 7
Parameters for HPLC method

| Column | Kinetex 2.6 μm EVO C18, 50 × 2.1 mm | |
|---|---|---|
| | (Phenomenex, Torrence, CA, USA) | |
| Mobile | A: 95% water + 5% Acetonitrile + 0.1% Trifluoroacetic | |
| phases | acid | |
| | B: 95% Acetonitrile + 5% water + 0.1% Trifluoroacetic | |
| | acid | |
| Gradient | Time [min] | B [%] |
| | 0 | 0 |
| | 3 | 60 |
| | 3.1 | 95 |
| | 4 | 95 |
| | 4.1 | 0 |
| | 5 | 0 |

| | |
|---|---|
| Temperature | 30° C. |
| Detection | ELSD (parameters: Evaporator 40° C./Nebuliser 40° C.) |
| Flowrate | 0.5 ml/min |
| Injection | 1 μL |

### EXAMPLES/COMBINATIONS

A. A method of making glycomonolipids, comprising:
(a) making glycomonolipids via a single catalytic step by contacting a hydroxy fatty acid with a glycosyl donor in the presence of an enzyme selected from
   i. a glycosyltransferase; and
   ii. glycosidases;
(b) optionally isolating the made glycomonolipids;
wherein the glycomonolipids have the following Formula 1:

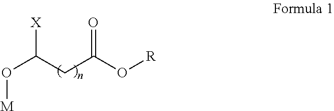

wherein n is from 0 to 15;
wherein X is selected from an alkyl, aryl, heteroalkyl, heteroaryl, unsaturated alkenyl, and unsaturated heteroalkenyl; and wherein X has a carbon chain length from 4 to 22;
wherein M is selected from one or two of glucose, sulfoquinovose, fructose, galactose, ribose, maltose, xylose, rhamnose, sophorose, mannose, arabinose, fucose, and combinations and stereoisomers thereof;
and wherein R is selected from —H, an alkyl selected from a straight-chained, branched or cyclic alkyl; a heteroalkyl; aryl; heteroaryl; hetero arylalkyl; arylalkyl; tauryl; and all possible stereoisomers thereof.
B. The method according to paragraph B, wherein the hydroxy fatty acid is a linear chain hydroxy fatty acid;
and wherein X is selected from an alkyl and unsaturated alkenyl and has a carbon chain length from 4 to 22.
C. The method according to paragraph A or B, wherein R is selected from —H and an alkyl and has a straight-chain carbon length from 1 to 4.
D. The method of any one of paragraphs A-C, wherein the glycosyl donor comprises at least one rhamnose.
E. The method of any one of paragraphs A-D, wherein the rhamnose is a nucleoside monophosphate (NMP) nucleoside diphosphate (NDP) or a nucleoside triphosphate (NTP).
F. The method of any one of paragraphs A-E, wherein the glycomonolipids are rhamnomonolipids.
G. The method of any one of paragraphs A-F, wherein the rhamnomonolipids are selected from Rha-C8, Rha-C10, Rha-C12, Rha-C14, RhaRha-C8, RhaRha-C10, RhaRha-C12and RhaRha-C14.
H. The method of any one of paragraphs A-G, wherein the enzyme is a glycosyltransferase that comprises a polypeptide sequence having at least 60% identity to SEQ ID NO: 1 through SEQ ID NO:12 and fragments thereof.
I. The method of any one of paragraphs A-H, wherein the glycosyltransferase comprises a polypeptide sequence having at least 75% identity to SEQ ID NO: 1 through SEQ ID NO:12 and fragments thereof.
J. The method of any one of paragraphs A-I, wherein the glycosyltransferase comprises a polypeptide sequence having at least 90% identity to SEQ ID NO: 1 through SEQ ID NO:12 and fragments thereof.
K. The method of any one of paragraphs A-J, wherein the enzyme is a glycosyltransferase that comprises at least one amino acid mutation selected from M1A, H2R, V3I, I4L, I4V, I5L, I5V, T6I, A7P, V8I, F17L, V18L, V18L, V18I, I20L, T23A, T23S, T23S, T23A, T23E, V25A, A26S, V31I, T32S, F33V, F33M, C34A, A35T, A35V, S36Y, S36N, S36F, A37P, A37EW, A37EF, A37PA, A37E, A37G, A37P, A38V, A38G, A38Y, A38R, F39L, F39A, A40K, A40R, A40P, A40E, A40S, P41E, P41S, P41A, L42S, L42A, I43A, I43V, I43L, E44D, E44G, R45L, R45G, R45E, C46S, C46A, C46P, C46R, G47A, F48M, F48L, N49S, N49A, N49E, N49T, N49G, N49G, N49K, N49E, N49R, N49S, F50V, F50L, L51I, L51V, L51V, L51V, P52A, L53V, L53R, L53I, G54T, T55E, T55S, T55P, R56A, R56A, R56A, R56P, E57N, E57D, E58G, E58Q, Y59F, Y59I, D60E, D60N, D60H, D60A, D60Q, D60Q, E61A, E61A, V62L, V62I, V62A, V62P, M63I, M63V, M63A, M63L, M63S, M63P, A64T, A64V, A64S, A64Y, D65S, D65N, A67R, H70N, H70D, P71S, R72K, R72L, R72E, T73K, T73A, T73G, T73D, S74A, L75F, P76R, P76G, T77V, L78M, K80R, K80R, T81A, T81W, T81V, I82V, I82V, A83G, S84G, S84P, S84T, S84G, T85S, T85A, L86I, E88D, E88P, E88A, Q89L, Q89H, Y90F, R91D, R91E, L92V, L92M, E94Q, E94A, Q95A, A96L, A96L, A96V, A96E, A96T, A96H, C97A, C97V, C97T, D98N, D99R, D99E, D99G, D99E, E100D, E100Q, V102I, M103L, L104I, L104V, L104I, L104V, L104V, G105A, S106H, S106N, S106R, L107S, L107Q, L107V, S111A, L115M, L115V, K118R, K118R, H119R, H119Y, I121A, I121V, V124A, V124I, T125S, A126G, A126V, A126G, Q127E, Q127H, V128A, V128L, V128I, V128A, S129T, S129Q, S129A, F131W, F131Q, F131H, G132N, G132Q, G132D, F133L, F133V, F133I, F133I, F133I, F133L, W134L, A136P, A136S, A137Q, A137K, A137F, A137L, A137R, A137H, A137M, A138S, A138D, S140A, S140P, S140P, S140P, T141V, T141I, T141V, H142L, H142S, P143-, P144A, P144K, P144K, P144L, G145P, G145R, A146T, A146F, A146T, A146L, A146S, A146L, N147T, N147P, N147S, L148I, L148V, A150E, A150T, A150G, F151S, F151E, F151D, F151T, F151W, F151W, V152L, V152L, P153T, P153S, Y154R, Y154D, Y154R, Y154V, Y154R, Y154L, P155E, V156I, V156P, V156D, V156Q, V156G, V156L, V156I, V156A, V156E, V156L, K157R, K157E, K157R, R158A, R158S, R158N, R158K, R158E, R158M, R158Q, L159E, L159D, L159G, L159R, L159A, L159K, L159T, L160F, L160I, L160A, L160V, L160Y, L161M, L161F, L161M, L161W, L161M, G162L, G162D, G162R, G162R, G162W, M163E, M163I, M163G, M163L, M163L, M163D, M163F, M163V, M163I, M163F, M163V, M163K, I164T, I164E, I164G, I164C, I164A, I164V, E165V, E165Y, E165D, E165A, E165I, R166K, R166L, R166E, R166V, R166Q, R166H, R166D, R166S, A167G, A167T, A167P, A167L, A167T, A167G, A167S, A167M, A167N, A167S, A167D, A167E, F168I, F168S, F168V, F168C, F168A, F168E, F168V, L169V, R171D, R171E, R171N, R171K, V172T, V172I, V172I, V172A, V172A, V172L, M173C, M173C, A174T, A174K, A174K, A174G, E176D, E176A, S179R, S179T, S179G, S179R, F180L, F180V, F180L, Q182S, Q182A, Q182R, Q182R, Q182G, Q182A, L186M, A187P, A187P, T189A, T189V, T189V, T189A, K190S, K190R, K190R, K190T, K190S, I192F, I193M, I193L, I193F, S194G, S194G, Q195E, Q195R, I197L, I197V, S198H, S198A, S198H, D201Q, R202G, R202K, R202G, E209D, E209D, V214P, V214T, V214A, H216T, H216R, H216P, H216Q, H216S, H216S, A220S, A220S, Q221H, Q221N, Q221R, T222A, T222I, V223S, V223A, F230Y, S233A, S233A, S233G, S233T, G234S, G234D, L235D, L235E, L235G, L235F, Q236H, Q236E, Q236L, Q236H, Q236R, Q236E, Q236L, P237T, P237T, P237Q, P237E, P237A, P237Q, P237Q, L238V, L238P, L238T, L238M, L238I, L238R, L238V, A240T, A240P, A240D, E243D, D244A, D244E, D244E, N247A, N247A, N247S, N247S, N247Q, N247R, E248G, E248A, E248A, A249G, A249E, A249T, A249G, A249S, S250P, S250P, S250P, V253I, T259A, T259S, M260N, V261R, V261I, V261L, V261I, N262D, N262D, N262H, N262D, N262D, N262D, G263A, G263A, G263S, G263P, G263S, G263E, G263E, E264G, E264P, E264Q, E264T, E264D, E264A, Q265A, Q265T, Q265R, Q265R, Q265S, Y266F, T268R, T268A, T268S, A269T, V271A, V271I, V271L, V271A, V271A, Q272E, Q272E, L274C, K275R, K275R, L276S, L276R, L276K, L276A, L276R, I277T, I277L, I277A, I277L, I277M, N278G, N278G, V2821, F283L, T285A, S286G, S286R, S286K, S286R, S286R, Q287N, Q287D, Q287E, Q287G, S288P, S288T, S288A, S288E, S288G, V289D, V289A, V289T, V289P, D290E, D290L, D290V, P291D, P291A, P291H, P291Q, S292V, S292P, S292T, S292A, S292E, V293P, V293L, V293L, V293D, V293P, V293A, S294P, S294A, S294L, S295R, S295T, S295A, S295L, E296S, E296A, E296S, E296D, E296G, E296P, G297S, G297S, G297D, G297S, G297P, G297D, G297P, R298Q, R298Q, R298N, R298D, R298E, I299V, V301L, R303S, R303P, V305A, S308T, R309K, R309L, R309A, R309S, I310L, H313Q, H313R, S314T, S314A, S314A, S314A, S314C, S314C, A315S, A315T, L317I, V323I, V323I, T326A, T326V, L328Q, M330I, M330L, M330I, M330L, M330F, M330L, V334I, I337L, I337V, I337L, A338V, A338V, F341L, F346L, M352L, M352V, M352I, E353Q, C357V, C357V, L359I, L359V, V361L, V361I, V361L, V361I, V361L, F362S, F362D, F362L, F362R, F362R, F362L, F362K, F362L, F362S, F362P, F362D, P363G, P363A, P363R, P363S, P364S, P364R, P364K, A365V, S366T, S366Q, A367G, A367G, A367T, E368R, E368Q, S369R, S369P, S369A, L370I, A371S, A371T, A371T, T372A, T372R, D375Q, D375Q, D375R, D375E, K376R, K376T, K376R, K376R, I377L, I377V, N379D, N379K, N379K, N379G, N379D, S380D, S380D, S380D, S380E, Q381P, E382A, E382Q, E382A, V383I, V383I, R384S, R384A, R384T, A385E, A385E, S386N, S386A, S386N, S386K, S386R, S386A, E388T, E388A, E388A, E388D, E388Q, E388R, R389Q, R389K, R389E, Y390M, Y390T, Y390M, Y390L, Y390I, R391A, T392Q, T392A, T392K, T392K, T392P, T392S, T392K, T392K, L393R, L393R, L393Q, I394V, I394M, I394L, I394F, I394M, P395A, S396T, G397S, G397A, D398P, D398E, D398E, D398E, S399A, S399T, S399R, A400G, S401L, S401C, S401C, S401A, E402Q, A403S, A403R, A403T, A403K, L406S, L406E, L406D, L406N, L406R, Q407F, Q407A, Q407F, I408V, A410T, A410S, L411V, L411M, L411F, L411I, A412V, A412S, R413S, R413P, R413T, R413A, T414R, T414P, T414L, T414S, T414E, A415P, A415K, A415R, A415S, R416A, R416I, R416T, R416K, R416G, S417R, S417Y, S417A, S417K, R418Q, R418T, A419T, A419T, A419V and A420R in the glycosyltransferase amino acid sequence of SEQ ID NO: 1.
L. The method of any one of paragraphs A-K, wherein the enzyme is a glycosyltransferase that comprises a deletion in at least one amino acid selected from E44, R45, C46, G47, F39, N49, F48, N49, F50, L51, P52, L53, G54, T55, R56, E57, E58, Y59, D60, E61, V62, P143, I164, D290, A415, R416, S417, R418, A419 and A420 in the glycosyltransferase amino acid sequence of SEQ ID NO: 1.
M. The method of any one of paragraphs A-L, wherein the enzyme is a glycosyltransferase that comprises an insertion to at least one amino acid selected from T392AV, I408VLS, S401CPA, E248AG, N247AG, R418APAG, E248GG, A420RVAA, R418VAAA, A37PT, P71GGTF, P71CGPGEGWW, P71GGRF, in the glycosyltransferase amino acid sequence of SEQ ID NO: 1.
N. The method of any one of paragraphs A-M, wherein the enzyme is a glycosyltransferase that is produced in a recombinant host cell.
O. The method of any one of paragraphs A-N, wherein the host cell is at least one of a eukaryotic or a prokaryotic organism.
P. The method of any one of paragraphs A-O, wherein the host cell is at least one of Escherichia coli, Bacillus subtilis, Streptomyces coelicolor, Pseudomonas aeruginosa, Pichia pastoris, Saccharomyces cerevisiae, Kluyveromyces lactis, Aspergillus niger, Trichoderma reesei, Corynebacterium spp., Pseudomonas putida, Burkholderia thailandensis, Parabulkholderia spp., Acinetobacter spp., Alcanivorax spp., Antarctobacter spp., Bacillus spp., Burkholderia spp., Candida apicola, Cellulomonas cellulans, Cupriavidus necator, Enterobacter spp., Halomonas spp., Lactobacillus spp., Marinobacter spp., Myxococcus spp., Nocardioides spp., Pseudoalteromonas spp., Pseudomonas spp., Pseudoxanthomonas spp., Renibacterium salmoninarum, Rhodoccus spp., Rhodotorula bogoriensis, Tetragenococcus koreensis, Methylobacterium spp., and Methylorubrum spp.
Q. The method of any one of paragraphs A-P, wherein the glycomonolipids produced are rhamnomonolipids.
R. The method of any one of paragraphs A-Q, wherein the rhamnomonolipids are produced in a cell-free system at a pH from 6 to 10; at a temperature from 25° C. to 42° C.; and optionally under agitation in a buffer solution.
S. The method of any one of paragraphs A-R, wherein the enzyme is a gylcosyltransferase that is encapsulated or immobilized.
T. The method of any one of paragraphs A-S, wherein the enzyme is a glycosidase that comprises a polypeptide sequence having at least 60% identity to SEQ ID NO: 30 through SEQ ID NO: 62 and 80 and fragments thereof.
U. The method of any one of paragraphs A-T, wherein the glycosyltransferase comprises a polypeptide sequence having at least 75% identity to SEQ ID NO: 30 through SEQ ID NO:62 and 80 and fragments thereof.
V. The method of any one of paragraphs A-U, wherein the glycosyltransferase comprises a polypeptide sequence having at least 90% identity to SEQ ID NO: 30 through SEQ ID NO: 62 and 80 and fragments thereof.
W. The method of any one of paragraphs A-V, wherein the enzyme is a glycosidase that is produced in a recombinant host cell.
X. The method of any one of paragraphs A-W, wherein the host cell is at least one of a eukaryotic or a prokaryotic organism.
Y. The method of any one of paragraphs A-X, wherein the host cell is at least one of Escherichia coli, Bacillus subtilis, Streptomyces coelicolor, Pseudomonas aeruginosa, Pichia pastoris, Saccharomyces cerevisiae, Kluyveromyces lactis, Aspergillus niger, Trichoderma reesei, Corynebacterium spp., Pseudomonas putida, Burkholderia thailandensis, Parabulkholderia spp., Acinetobacter spp., Alcanivorax spp., Antarctobacter spp., Bacillus spp., Burkholderia spp., Candida apicola, Cellulomonas cellulans, Cupriavidus necator, Enterobacter spp., Halomonas spp., Lactobacillus spp., Marinobacter spp., Myxococcus spp., Nocardioides spp., Pseudoalteromonas spp., Pseudomonas spp., Pseudoxanthomonas spp., Renibacterium salmoninarum, Rhodoccus spp., Rhodotorula bogoriensis, Tetragenococcus koreensis, Methylobacterium spp., and Methylorubrum spp.
Z. The method of any one of paragraphs A-Y, wherein the glycosidase comprises amino acids selected from E206, E387, H150, N205, R79, Q18, E432, W425, Y321, Y322, F222, W361, F441, V209, F359, Not D or E in 341, wherein said positions are numbered with reference to SEQ ID NO: 83.
AA. The method of any one of paragraphs A-Z, wherein the glycosidase comprises amino acids selected from E572, E841, D567, H862, N849, R858, D579, W864, W576, W679, W684, wherein said positions are numbered with reference to SEQ ID NO: 84.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

Every document cited herein, including any cross referenced or related patent or application, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests, or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A method of making glycomonolipids, comprising:
(a) making glycomonolipids via a single catalytic step by contacting a hydroxy fatty acid with a glycosyl donor in the presence of an enzyme selected from
i) a glycosyltransferase; and
ii) glycosidases;
(b) optionally isolating the made glycomonolipids;
wherein the glycomonolipids have the following Formula 1: 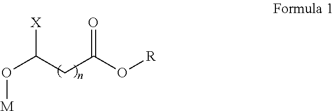
wherein n is from 0 to 15;
wherein X is selected from an alkyl, aryl, heteroalkyl, heteroaryl, unsaturated alkenyl, and unsaturated heteroalkenyl, and combinations and stereoisomers thereof; and wherein X has a carbon chain length from 4 to 22;
wherein M is selected from one or two of glucose, sulfoquinovose, fructose, galactose, ribose, maltose, xylose, rhamnose, sophorose, mannose, arabinose, fucose, and combinations and stereoisomers thereof;
and wherein R is selected from —H, an alkyl selected from a straight-chained, branched or cyclic alkyl; a heteroalkyl; aryl; heteroaryl; hetero arylalkyl; arylalkyl; tauryl; and all possible stereoisomers and combinations thereof.

2. The method of claim 1, wherein the hydroxy fatty acid is a linear chain hydroxy fatty acid;
and wherein X is selected from an alkyl and unsaturated alkenyl and has a carbon chain length from 4 to 22.

3. The method of claim 2, wherein R is selected from —H and an alkyl and has a straight-chain carbon length from 1 to 4.

4. The method of claim 1, wherein the glycosyl donor comprises at least one rhamnose.

5. The method of claim 4, wherein the rhamnose is a nucleoside monophosphate (NMP) nucleoside diphosphate (NDP) or a nucleoside triphosphate (NTP).

6. The method of claim 1, wherein the glycomonolipids are rhamnomonolipids.

7. The method of claim 6, wherein the rhamnomonolipids are selected from Rha-C8, Rha-C10, Rha-C12, Rha-C14, RhaRha-C8, RhaRha-C10, RhaRha-C12 and RhaRha-C14.

8. The method of claim 1, wherein the enzyme is a glycosyltransferase that comprises a polypeptide sequence having at least 60% identity to any one of SEQ ID NO: 1 through SEQ ID NO: 12 and fragments thereof.

9. The method of claim 1, wherein the glycosyltransferase comprises a polypeptide sequence having at least 75% identity to any one of SEQ ID NO: 1 through SEQ ID NO: 12 and fragments thereof.

10. The method of claim 1, wherein the glycosyltransferase comprises a polypeptide sequence having at least 90% identity to any one of SEQ ID NO: 1 through SEQ ID NO: 12 and fragments thereof.

11. The method of claim 1, wherein the enzyme is a glycosyltransferase that comprises at least one amino acid mutation selected from M1A, H2R, V3I, I4L, I4V, I5L, 15V, T6I, A7P, V8I, F17L, V18L, V18L, V18I, I20L, T23A, T23S, T23S, T23A, T23E, V25A, A26S, V31I, T32S, F33V, F33M, C34A, A35T, A35V, S36Y, S36N, S36F, A37P, A37EW, A37EF, A37PA, A37E, A37G, A37P, A38V, A38G, A38Y, A38R, F39L, F39A, A40K, A40R, A40P, A40E, A40S, P41E, P41S, P41A, L42S, L42A, I43A, I43V, I43L, E44D, E44G, R45L, R45G, R45E, C46S, C46A, C46P, C46R, G47A, F48M, F48L, N49S, N49A, N49E, N49T, N49G, N49G, N49K, N49E, N49R, N49S, F50V, F50L, L51I, L51V, L51V, L51V, P52A, L53V, L53R, L53I, G54T, T55E, T55S, T55P, R56A, R56A, R56A, R56P, E57N, E57D, E58G, E58Q, Y59F, Y59I, D60E, D60N, D60H, D60A, D60Q, D60Q, E61A, E61A, V62L, V62I, V62A, V62P, M63I, M63V, M63A, M63L, M63S, M63P, A64T, A64V, A64S, A64Y, D65S, D65N, A67R, H70N, H70D, P71S, R72K, R72L, R72E, T73K, T73A, T73G, T73D, S74A, L75F, P76R, P76G, T77V, L78M, K80R, K80R, T81A, T81W, T81V, I82V, I82V, A83G, S84G, S84P, S84T, S84G, T85S, T85A, L86I, E88D, E88P, E88A, Q89L, Q89H, Y90F, R91D, R91E, L92V, L92M, E94Q, E94A, Q95A, A96L, A96L, A96V, A96E, A96T, A96H, C97A, C97V, C97T, D98N, D99R, D99E, D99G, D99E, E100D, E100Q, V102I, M103L, L104I, L104V, L104I, L104V, L104V, G105A, S106H, S106N, S106R, L107S, L107Q, L107V, S111A, L115M, L115V, K118R, K118R, H119R, H119Y, I121A, I121V, V124A, V124I, T125S, A126G, A126V, A126G, Q127E, Q127H, V128A, V128L, V128I, V128A, S129T, S129Q, S129A, F131W, F131Q, F131H, G132N, G132Q, G132D, F133L, F133V, F133I, F133I, F133I, F133L, W134L, A136P, A136S, A137Q, A137K, A137F, A137L, A137R, A137H, A137M, A138S, A138D, S140A, S140P, S140P, S140P, T141V, T141I, T141V, H142L, H142S, P143-, P144A, P144K, P144K, P144L, G145P, G145R, A146T, A146F, A146T, A146L, A146S, A146L, N147T, N147P, N147S, L148I, L148V, A150E, A150T, A150G, F151S, F151E, F151D, F151T, F151W, F151W, V152L, V152L, P153T, P153S, Y154R, Y154D, Y154R, Y154V, Y154R, Y154L, P155E, V156I, V156P, V156D, V156Q, V156G, V156L, V156I, V156A, V156E, V156L, K157R, K157E, K157R, R158A, R158S, R158N, R158K, R158E, R158M, R158Q, L159E, L159D, L159G, L159R, L159A, L159K, L159T, L160F, L160I, L160A, L160V, L160Y, L161M, L161F, L161M, L161W, L161M, G162L, G162D, G162R, G162R, G162W, M163E, M163I, M163G, M163L, M163L, M163D, M163F, M163V, M163I, M163F, M163V, M163K, I164T, I164E, I164G, I164C, I164A, I164V, E165V, E165Y, E165D, E165A, E165I, R166K, R166L, R166E, R166V, R166Q, R166H, R166D, R166S, A167G, A167T, A167P, A167L, A167T, A167G, A167S, A167M, A167N, A167S, A167D, A167E, F168I, F168S, F168V, F168C, F168A, F168E, F168V, L169V, R171D, R171E, R171N, R171K, V172T, V172I, V172I, V172A, V172A, V172L, M173C, M173C, A174T, A174K, A174K, A174G, E176D, E176A, S179R, S179T, S179G, S179R, F180L, F180V, F180L, Q182S, Q182A, Q182R, Q182R, Q182G, Q182A, L186M, A187P, A187P, T189A, T189V, T189V, T189A, K190S, K190R, K190R, K190T, K190S, I192F, I193M, I193L, I193F, S194G, S194G, Q195E, Q195R, I197L, I197V, S198H, S198A, S198H, D201Q, R202G, R202K, R202G, E209D, E209D, V214P, V214T, V214A, H216T, H216R, H216P, H216Q, H216S, H216S, A220S, A220S, Q221H, Q221N, Q221R, T222A, T222I, V223S, V223A, F230Y, S233A, S233A, S233G, S233T, G234S, G234D, L235D, L235E, L235G, L235F, Q236H, Q236E, Q236L, Q236H, Q236R, Q236E, Q236L, P237T, P237T, P237Q, P237E, P237A, P237Q, P237Q, L238V, L238P, L238T, L238M, L238I, L238R, L238V, A240T, A240P, A240D, E243D, D244A, D244E, D244E, N247A, N247A, N247S, N247S, N247Q, N247R, E248G, E248A, E248A, A249G, A249E, A249T, A249G, A249S, S250P, S250P, S250P, V253I, T259A, T259S, M260N, V261R, V261I, V261L, V261I, N262D, N262D, N262H, N262D, N262D, N262D, G263A, G263A, G263S, G263P, G263S, G263E, G263E, E264G, E264P, E264Q, E264T, E264D, E264A, Q265A, Q265T, Q265R, Q265R, Q265S, Y266F, T268R, T268A, T268S, A269T, V271A, V271I, V271L, V271A, V271A, Q272E, Q272E, L274C, K275R, K275R, L276S, L276R, L276K, L276A, L276R, I277T, I277L, I277A, I277L, I277M, N278G, N278G, V282I, F283L, T285A, S286G, S286R, S286K, S286R, S286R, Q287N, Q287D, Q287E, Q287G, S288P, S288T, S288A, S288E, S288G, V289D, V289A, V289T, V289P, D290E, D290L, D290V, P291D, P291A, P291H, P291Q, S292V, S292P, S292T, S292A, S292E, V293P, V293L, V293L, V293D, V293P, V293A, S294P, S294A, S294L, S295R, S295T, S295A, S295L, E296S, E296A, E296S, E296D, E296G, E296P, G297S, G297S, G297D, G297S, G297P, G297D, G297P, R298Q, R298Q, R298N, R298D, R298E, I299V, V301L, R303S, R303P, V305A, S308T, R309K, R309L, R309A, R309S, I310L, H313Q, H313R, S314T, S314A, S314A, S314A, S314C, S314C, A315S, A315T, L317I, V323I, V323I, T326A, T326V, L328Q, M330I, M330L, M330I, M330L, M330F, M330L, V334I, I337L, I337V, I337L, A338V, A338V, F341L, F346L, M352L, M352V, M352I, E353Q, C357V, C357V, L359I, L359V, V361L, V361I, V361L, V361I, V361L, F362S, F362D, F362L, F362R, F362R, F362L, F362K, F362L, F362S, F362P, F362D, P363G, P363A, P363R, P363S, P364S, P364R, P364K, A365V, S366T, S366Q, A367G, A367G, A367T, E368R, E368Q, S369R, S369P, S369A, L370I, A371S, A371T, A371T, T372A, T372R, D375Q, D375Q, D375R, D375E, K376R, K376T, K376R, K376R, I377L, I377V, N379D, N379K, N379K, N379G, N379D, S380D, S380D, S380D, S380E, Q381P, E382A, E382Q, E382A, V383I, V383I, R384S, R384A, R384T, A385E, A385E, S386N, S386A, S386N, S386K, S386R, S386A, E388T, E388A, E388A, E388D, E388Q, E388R, R389Q, R389K, R389E, Y390M, Y390T, Y390M, Y390L, Y390I, R391A, T392Q, T392A, T392K, T392K, T392P, T392S, T392K, T392K, L393R, L393R, L393Q, I394V, I394M, I394L, I394F, I394M, P395A, S396T, G397S, G397A, D398P, D398E, D398E, D398E, S399A, S399T, S399R, A400G, S401L, S401C, S401C, S401A, E402Q, A403S, A403R, A403T, A403K, L406S, L406E, L406D, L406N, L406R, Q407F, Q407A, Q407F, I408V, A410T, A410S, L411V, L411M, L411F, L411I, A412V, A412S, R413S, R413P, R413T, R413A, T414R, T414P, T414L, T414S, T414E, A415P, A415K, A415R, A415S, R416A, R416I, R416T, R416K, R416G, S417R, S417Y, S417A, S417K, R418Q, R418T, A419T, A419T, A419V and A420R in the glycosyltransferase amino acid sequence of SEQ ID NO: 1.

12. The method of claim 1, wherein the enzyme is a glycosyltransferase that comprises a deletion in at least one amino acid selected from E44, R45, C46, G47, F39, N49, F48, N49, F50, L51, P52, L53, G54, T55, R56, E57, E58, Y59, D60, E61, V62, P143, I164, D290, A415, R416, S417, R418, A419 and A420 in the glycosyltransferase amino acid sequence of SEQ ID NO: 1.

13. The method of claim 1, wherein the enzyme is a glycosyltransferase that comprises an insertion to at least one amino acid selected from T392AV, I408VLS, S401CPA, E248AG, N247AG, R418APAG, E248GG, A420RVAA, R418VAAA, A37PT, P71GGTF, P71CGPGEGWW, P71GGRF, in the glycosyltransferase amino acid sequence of SEQ ID NO: 1.

14. The method of claim 1, wherein the enzyme is a glycosyltransferase that is produced in a recombinant host cell.

15. The method of claim 14, wherein the host cell is at least one of a eukaryotic or a prokaryotic organism.

16. The method of claim 14, wherein the host cell is at least one of Escherichia coli, Bacillus subtilis, Streptomyces coelicolor, Pseudomonas aeruginosa, Pichia pastoris, Saccharomyces cerevisiae, Kluyveromyces lactis, Aspergillus niger, Trichoderma reesei, Corynebacterium spp., Pseudomonas putida, Burkholderia thailandensis, Parabulkholderia spp., Acinetobacter spp., Alcanivorax spp., Antarctobacter spp., Bacillus spp., Burkholderia spp., Candida apicola, Cellulomonas cellulans, Cupriavidus necator, Enterobacter spp., Halomonas spp., Lactobacillus spp., Marinobacter spp., Myxococcus spp., Nocardioides spp., Pseudoalteromonas spp., Pseudomonas spp., Pseudoxanthomonas spp., Renibacterium salmoninarum, Rhodoccus spp., Rhodotorula bogoriensis, Tetragenococcus koreensis, Methylobacterium spp., and Methylorubrum spp.

17. The method of claim 14, wherein the glycomonolipids produced are rhamnomonolipids.

18. The method of claim 1, wherein the rhamnomonolipids are produced in a cell-free system at a pH from 6 to 10; at a temperature from 25° C. to 42° C.; and optionally under agitation in a buffer solution.

19. The method of claim 1, wherein the enzyme is encapsulated or immobilized.

20. The method of claim 1, wherein the enzyme is a glycosidase that comprises a polypeptide sequence having at least 60% identity to SEQ ID NO: 30 through SEQ ID NO: 62 and 80 and fragments thereof.

21. The method of claim 1, wherein the glycosidase comprises a polypeptide sequence having at least 75% identity to SEQ ID NO: 30 through SEQ ID NO: 62 and 80 and fragments thereof.

22. The method of claim 1, wherein the glycosidase comprises a polypeptide sequence having at least 90% identity to SEQ ID NO: 30 through SEQ ID NO: 62 and 80 and fragments thereof.

23. The method of claim 1, wherein the enzyme is a glycosidase that is produced in a recombinant host cell.

24. The method of claim 23, wherein the host cell is at least one of a eukaryotic or a prokaryotic organism.

25. The method of claim 24, wherein the host cell is at least one of Escherichia coli, Bacillus subtilis, Streptomyces coelicolor, Pseudomonas aeruginosa, Pichia pastoris, Saccharomyces cerevisiae, Kluyveromyces lactis, Aspergillus niger, Trichoderma reesei, Corynebacterium spp., Pseudomonas putida, Burkholderia thailandensis, Parabulkholderia spp., Acinetobacter spp., Alcanivorax spp., Antarctobacter spp., Bacillus spp., Burkholderia spp., Candida apicola, Cellulomonas cellulans, Cupriavidus necator, Enterobacter spp., Halomonas spp., Lactobacillus spp., Marinobacter spp., Myxococcus spp., Nocardioides spp., Pseudoalteromonas spp., Pseudomonas spp., Pseudoxanthomonas spp., Renibacterium salmoninarum, Rhodoccus spp., Rhodotorula bogoriensis, Tetragenococcus koreensis, Methylobacterium spp., and Methylorubrum spp.

26. The method of claim 23 wherein the glycosidase comprises amino acids selected from E206, E387, H150, N205, R79, Q18, E432, W425, Y321, Y322, F222, W361, F441, V209, F359, Not D or E in 341, wherein said positions are numbered with reference to SEQ ID NO: 83.

27. The method of claim 23 wherein the glycosidase comprises amino acids selected from E572, E841, D567, H862, N849, R858, D579, W864, W576, W679, W684, wherein said positions are numbered with reference to SEQ ID NO: 84.

28. A glycosyltransferase variant that comprises a mutation in at least one amino acid selected from M1A, H2R, V3I, I4L, I4V, I5L, I5V, T6I, A7P, V8I, F17L, V18L, V18L, V18I, I20L, T23A, T23S, T23S, T23A, T23E, V25A, A26S, V31I, T32S, F33V, F33M, C34A, A35T, A35V, S36Y, S36N, S36F, A37P, A37EW, A37EF, A37PA, A37E, A37G, A37P, A38V, A38G, A38Y, A38R, F39L, F39A, A40K, A40R, A40P, A40E, A40S, P41E, P41S, P41A, L42S, L42A, I43A, I43V, I43L, E44D, E44G, R45L, R45G, R45E, C46S, C46A, C46P, C46R, G47A, F48M, F48L, N49S, N49A, N49E, N49T, N49G, N49G, N49K, N49E, N49R, N49S, F50V, F50L, L51I, L51V, L51V, L51V, P52A, L53V, L53R, L53I, G54T, T55E, T55S, T55P, R56A, R56A, R56A, R56P, E57N, E57D, E58G, E58Q, Y59F, Y59I, D60E, D60N, D60H, D60A, D60Q, D60Q, E61A, E61A, V62L, V62I, V62A, V62P, M63I, M63V, M63A, M63L, M63S, M63P, A64T, A64V, A64S, A64Y, D65S, D65N, A67R, H70N, H70D, P71S, R72K, R72L, R72E, T73K, T73A, T73G, T73D, S74A, L75F, P76R, P76G, T77V, L78M, K80R, K80R, T81A, T81W, T81V, I82V, I82V, A83G, S84G, S84P, S84T, S84G, T85S, T85A, L86I, E88D, E88P, E88A, Q89L, Q89H, Y90F, R91D, R91E, L92V, L92M, E94Q, E94A, Q95A, A96L, A96L, A96V, A96E, A96T, A96H, C97A, C97V, C97T, D98N, D99R, D99E, D99G, D99E, E100D, E100Q, V102I, M103L, L104I, L104V, L104I, L104V, L104V, G105A, S106H, S106N, S106R, L107S, L107Q, L107V, S111A, L115M, L115V, K118R, K118R, H119R, H119Y, I121A, I121V, V124A, V124I, T125S, A126G, A126V, A126G, Q127E, Q127H, V128A, V128L, V128I, V128A, S129T, S129Q, S129A, F131W, F131Q, F131H, G132N, G132Q, G132D, F133L, F133V, F133I, F133I, F133I, F133L, W134L, A136P, A136S, A137Q, A137K, A137F, A137L, A137R, A137H, A137M, A138S, A138D, S140A, S140P, S140P, S140P, T141V, T141I, T141V, H142L, H142S, P143-, P144A, P144K, P144K, P144L, G145P, G145R, A146T, A146F, A146T, A146L, A146S, A146L, N147T, N147P, N147S, L148I, L148V, A150E, A150T, A150G, F151S, F151E, F151D, F151T, F151W, F151W, V152L, V152L, P153T, P153S, Y154R, Y154D, Y154R, Y154V, Y154R, Y154L, P155E, V156I, V156P, V156D, V156Q, V156G, V156L, V156I, V156A, V156E, V156L, K157R, K157E, K157R, R158A, R158S, R158N, R158K, R158E, R158M, R158Q, L159E, L159D, L159G, L159R, L159A, L159K, L159T, L160F, L160I, L160A, L160V, L160Y, L161M, L161F, L161M, L161W, L161M, G162L, G162D, G162R, G162R, G162W, M163E, M163I, M163G, M163L, M163L, M163D, M163F, M163V, M163I, M163F, M163V, M163K, I164T, I164E, I164G, I164C, I164A, I164V, E165V, E165Y, E165D, E165A, E165I, R166K, R166L, R166E, R166V, R166Q, R166H, R166D, R166S, A167G, A167T, A167P, A167L, A167T, A167G, A167S, A167M, A167N, A167S, A167D, A167E, F168I, F168S, F168V, F168C, F168A, F168E, F168V, L169V, R171D, R171E, R171N, R171K, V172T, V172I, V172I, V172A, V172A, V172L, M173C, M173C, A174T, A174K, A174K, A174G, E176D, E176A, S179R, S179T, S179G, S179R, F180L, F180V, F180L, Q182S, Q182A, Q182R, Q182R, Q182G, Q182A, L186M, A187P, A187P, T189A, T189V, T189V, T189A, K190S, K190R, K190R, K190T, K190S, I192F, I193M, I193L, 1193F, S194G, S194G, Q195E, Q195R, I197L, I197V, S198H, S198A, S198H, D201Q, R202G, R202K, R202G, E209D, E209D, V214P, V214T, V214A, H216T, H216R, H216P, H216Q, H216S, H216S, A220S, A220S, Q221H, Q221N, Q221R, T222A, T222I, V223S, V223A, F230Y, S233A, S233A, S233G, S233T, G234S, G234D, L235D, L235E, L235G, L235F, Q236H, Q236E, Q236L, Q236H, Q236R, Q236E, Q236L, P237T, P237T, P237Q, P237E, P237A, P237Q, P237Q, L238V, L238P, L238T, L238M, L2381, L238R, L238V, A240T, A240P, A240D, E243D, D244A, D244E, D244E, N247A, N247A, N247S, N247S, N247Q, N247R, E248G, E248A, E248A, A249G, A249E, A249T, A249G, A249S, S250P, S250P, S250P, V253I, T259A, T259S, M260N, V261R, V261I, V261L, V261I, N262D, N262D, N262H, N262D, N262D, N262D, G263A, G263A, G263S, G263P, G263S, G263E, G263E, E264G, E264P, E264Q, E264T, E264D, E264A, Q265A, Q265T, Q265R, Q265R, Q265S, Y266F, T268R, T268A, T268S, A269T, V271A, V271I, V271L, V271A, V271A, Q272E, Q272E, L274C, K275R, K275R, L276S, L276R, L276K, L276A, L276R, I277T, I277L, I277A, I277L, I277M, N278G, N278G, V2821, F283L, T285A, S286G, S286R, S286K, S286R, S286R, Q287N, Q287D, Q287E, Q287G, S288P, S288T, S288A, S288E, S288G, V289D, V289A, V289T, V289P, D290E, D290L, D290V, P291D, P291A, P291H, P291Q, S292V, S292P, S292T, S292A, S292E, V293P, V293L, V293L, V293D, V293P, V293A, S294P, S294A, S294L, S295R, S295T, S295A, S295L, E296S, E296A, E296S, E296D, E296G, E296P, G297S, G297S, G297D, G297S, G297P, G297D, G297P, R298Q, R298Q, R298N, R298D, R298E, I299V, V301L, R303S, R303P, V305A, S308T, R309K, R309L, R309A, R309S, I310L, H313Q, H313R, S314T, S314A, S314A, S314A, S314C, S314C, A315S, A315T, L317I, V323I, V323I, T326A, T326V, L328Q, M330I, M330L, M330I, M330L, M330F, M330L, V334I, I337L, I337V, I337L, A338V, A338V, F341L, F346L, M352L, M352V, M352I, E353Q, C357V, C357V, L359I, L359V, V361L, V361I, V361L, V361I, V361L, F362S, F362D, F362L, F362R, F362R, F362L, F362K, F362L, F362S, F362P, F362D, P363G, P363A, P363R, P363S, P364S, P364R, P364K, A365V, S366T, S366Q, A367G, A367G, A367T, E368R, E368Q, S369R, S369P, S369A, L370I, A371S, A371T, A371T, T372A, T372R, D375Q, D375Q, D375R, D375E, K376R, K376T, K376R, K376R, I377L, I377V, N379D, N379K, N379K, N379G, N379D, S380D, S380D, S380D, S380E, Q381P, E382A, E382Q, E382A, V383I, V383I, R384S, R384A, R384T, A385E, A385E, S386N, S386A, S386N, S386K, S386R, S386A, E388T, E388A, E388A, E388D, E388Q, E388R, R389Q, R389K, R389E, Y390M, Y390T, Y390M, Y390L, Y390I, R391A, T392Q, T392A, T392K, T392K, T392P, T392S, T392K, T392K, L393R, L393R, L393Q, I394V, I394M, I394L, I394F, I394M, P395A, S396T, G397S, G397A, D398P, D398E, D398E, D398E, S399A, S399T, S399R, A400G, S401L, S401C, S401C, S401A, E402Q, A403S, A403R, A403T, A403K, L406S, L406E, L406D, L406N, L406R, Q407F, Q407A, Q407F, I408V, A410T, A410S, L411V, L411M, L411F, L411I, A412V, A412S, R413S, R413P, R413T, R413A, T414R, T414P, T414L, T414S, T414E, A415P, A415K, A415R, A415S, R416A, R416I, R416T, R416K, R416G, S417R, S417Y, S417A, S417K, R418Q, R418T, A419T, A419T, A419V and A420R in the glycosyltransferase amino acid sequence of SEQ ID NO: 1.

29. A glycosyltransferase variant that comprises a deletion in at least one amino acid selected from E44, R45, C46, G47, F39, N49, F48, N49, F50, L51, P52, L53, G54, T55, R56,E57, E58, Y59, D60, E61, V62, P143, 1164, D290, A415, R416, S417, R418, A419 and A420 in the glycosyltransferase amino acid sequence of SEQ ID NO: 1.

30. A glycosyltransferase variant that comprises an insertion to at least one amino acid selected from T392AV, I408VLS, S401CPA, E248AG, N247AG, R418APAG, E248GG, A420RVAA, R418VAAA, A37PT, P71GGTF, P71CGPGEGWW, and P71GGRF in the glycosyltransferase amino acid sequence of SEQ ID NO: 1.


SEQUENCE LISTING (84 sequences)

SEQ ID NO: 1 - protein, 420 aa, synthetic construct
MHVIITAVGSAGDVHPFVGIGRTLVARGHRVTFCASAAFAPLIERCGFNFLPLGTREEYDEVMADPALWHPRTSLPTLWKTIASTLREQYRLLEQACDDETVMLGSLWAFSARLLQEKHGIPLVTAQVSPFGFWSAAAPSTHPPGANLPAFVPYPVKRLLLGMIERAFLDRVMAPELNSFRQELGLAPTKRIISQWISSPDRVLGLFPEWFAPVQHDWPAQTVLTGFPLFDESGLQPLDAELEDFLNEASPVVFTPGSTMVNGEQYFTAAVQALKLINRRGVFLTSQSVDPSVSSEGRILVRRYVPMSRILPHSAALVHHGGVGTTALAMAAGVPQIATPFAHDQFDNAARMERLGCGLRVFPPASAESLASALDKILNSQEVRASCERYRTLIPSGDSASEAAALQIEALARTARSRAA

SEQ ID NO: 2 - protein, 421 aa, synthetic construct
MNVFLATLGSSGDVFPFILIGEHMAKRGHKVFLCTNPYFKGLAESRGLNFISVGSTQEYIAGVHSRALWDKKTALDTLCRYMLNQQEEMYQQLERVIDSSSVLLTSLWSFSAKALGEKKGCCTLPVRVGPSTFLSCYNLPQHGKLALANKLPMPLRKGMVSLFEKKVLDKRLCAEVNAQRSRVGLPPIKRILSQWVHSSADGLICLFPEWFVHPQPDWPRNANLVGLPLFEAQDAPADEELARFLESETVMFSPGWSLQFDEYFFNAVIAEVTRQRRQCLVVGVNPDDVPVRPGVLVRKEVDLESCLRHCVAAVHHGGIGTISHCFYQSVPQLIFPSAFDQFDNAYRVEQLGCGIQAKSRLPEKFSDDLTRVMTDENMRKQCRRIQEKYGEKNHCLENVRHALEQAHQRHAPTPQCRLQMA

SEQ ID NO: 3 - protein, 412 aa, synthetic construct
MNLFLATLGSSGDVFPFILIGEHMARRGHKVFLCTNPYFKDLAESRGLNFISVGSTQEYISGVHSRALWDKKTALDTLCRYMLKQQDEMYQQLARVVDSSSVLLSSLWSFSAKILGEQKGCCTLPVRVGPSTFLSCYSLPQHGKLALANKLPMTLRKAVVSLFETKVLDKRFCAEVNALRSRVGLAPVKRILSQWVHTSPDGLICLFPEWFAHPQPDWPRNANLVGLPLFEAENAPVDEELGRFLERETVLFSPGWSLQFDEYFINAVIAEVTRQGRQCLVVGVNPEDVPVRPGVLVKKEVDLTACLRHCVAAVHHGGIGTLSHCFYQSIPQLIFPSAFDQFDNAHRVEQLGCGIQAKSRLPETFSDDLARVMTDENMRERCRRIQEKYGDKNHCLDNVRHALEQAHQRHAS

SEQ ID NO: 4 - protein, 420 aa, synthetic construct
MHVIITAVGSAGDVHPFVGIGRTLVARGHRVTFCASAAFAPLIERCGFNFLPLGTREEYDEVMADPALWHPRTSLPTLWKTIASTLREQYRLLEQACDDETVMLGSLWAFSARLLQEKHGVPLITAQVSPFGFWSAAAPSTHPPGANLPAFVPYPVKRLLLGMIERAFLDRVMAPELNSFRRELGLAPTKRIISQWISSPDRVLGLFPEWFAPVQHDWPAQTVLTGFPLFDESGLQPLDAELEDFLNEASPVVFTPGSTMVNGEQYFTAAVQALKLVNRRGVFLTSQSVGPSVSGEGRILVRRYVPMSRILPHSAALVHHGGVGTTALAMAAGVPQIATPFAHDQFDNAARMERLGCGMRVFPPASAESLAAALDKVLNSQEVRASCDRYRTLIPSGDSASEAAALQIEALARSARSRAA

SEQ ID NO: 5 - protein, 420 aa, synthetic construct
MNLFLATLGSSGDVFPFIIIGEHMAKRGHKVFLCTNPYFKCLAESRGLEFISVGSAQEYIAGVHSRALWDSETALDTLCRYMLKQQDEMYQQLERVVDGSSVLLTSLWSFSAKTLGERKGCCTLPVRVGPSTFLSCYNLPQHGKLALANTLPMTLRKGIVSLFEKQVLDKRFCAEVNTLRSRVGLAPVKRILSQWVHTSADGLICLFPEWFAHPQPDWPRNANLVGLPLFEAENAPVDEELGRFLERETVLFSPGWSLQFDEYFFNAIVAEVTRQRRQCLVVGVNPEDVPVRPGVLVKKEVDLAACLRHCVAAVHHGGIGTISHCFYQSVPQLIFPSAFDQFDNAYRVEQLGCGIQAKSRLPEKFSDALDRVMTDENMRERCRRIQEKYGDKNHCLENVRHALEQAHQRHASCPGSVAYR

SEQ ID NO: 6 - protein, 451 aa, synthetic construct
MNILLNPAGSSGDVLPFIRLGMALKARGHDVTVITMGRYESLVRHANLAFVEWISPEAMQAIEDNPDLWHPTRGLLLAIRTLEPWYAQLHEHIQARYVPGQTVLVAGSLNLAARLAHDRLGVPLATLLLQPAYLRSVHRSPVLPGVPTPDWAPRIWKRLVYFLSDALVLDWVAAPAVNALRAELGLPPARGVLGSWWLSPQRVIGLFPDWFGEPQPDWPAQVRLTDFPLYDGLGAEPVPAGLDEFLENGEPPVVFTPGTMMRHPRRFFAEAVDACGRLGRRGLLLTRYRDQLPARLPSGVCHFDYVPLSHVLPRAAALVSHGGIGTVSQALVAGTPQLIMPLGFDQFDNAARLEHMGVATTLAPRAFRGRAVAQRVARLLDSATVARACKAAAVRLHKDRWVGPACQAVEELAGEDGAAQLPGSSGCFPARQASHPDPKAVEDYLIACALP

SEQ ID NO: 7 - protein, 427 aa, synthetic construct
MQILMIAIGSAGDVHPLLAVGRELANRGNRVTVFTNPAFQPLVERCGLGFYALGTAQEYHRAIETPALWNPSTSFATLWRIVSPVLRPMVEGLLATIDDETVMVGSLWAFGARIVNELYDVPLVTAQVSASTFLSAQLPPVHKRVRIPAGLPYGVKSGLLWLIERAALDRVCAPALNELRGELGLAPVSRVLGRWMHSPHCTLGLFPDWFAPPQHDWPLHVTLTGFPLFDEPERSVADPELGEFLEAGSPPVVFTQGSTSVSGPLYVETALKTLKHLGLRGLFLGGDAGQLPQGDGILHRRFAPLASVLPKSAAMVHHGGIGTMSFAFAAGIPQLATPFAHDQFDNAARLARLGCGVRRDWPVDSSSMASTLQALLESPEVRDACRTVRRRMKPAHVARADAAGMIERCGLAARPRVLAVSQAEHNV

SEQ ID NO: 8 - protein, 426 aa, synthetic construct
MHAILIAIGSAGDVFPFIGLARTLKLRGHRVSLCSIPVFRAAVEQHGIEFVPLSDELTYRRTMGDPRLWDPKTSFGVLWQAIAGMIEPVYEYVCAQRHDDIVVVGSLWALGARIAQEKYGIPYLSVQVSPSTLLSAHLPPVHPRFNVPEQVPLAMRKLLWRCIERFKLDRTCAPEINAVRRKAGLVGPAKRIFTQWMHSPQGVLCLFPAWFAPPQQDWPQPLHMTGFPLFDGSVPGTRLDDELQRFLEQGSRPLVFTQGSTEHLQGDFYAMALRALERLGARGIFLTGAGQEPLRGLPSHVLQRSYVPLGALLPACAGLVHPAGIGAMSLALAAGVPQVLLPCAHDQFDNAERLVRLGCGIRLGLPLREQALRESLWRLLEDPALAAACRRFMELSQPHSIACGKAAQVVERCHREGDVRWLKAAS

SEQ ID NO: 9 - protein, 430 aa, synthetic construct
MHILIIAIGSAGDVHPFLGLARTFVKQGHRVSFCTSPAFAPVVERCGLRMLPLGTAAEYQAAMNDPALWDPRTSFKVLWKIMAGYIRPLFDILEAEIDNETVLVGSLWAFGARLMQEKYGVPYVSVQVSPSTFLSAKLPPVHKRFTIPSFLPSSLRAALLHTIERHVIDPVCAPDLNRVRDNLGLPPVQSILGQWVHSPQGVLGLFPAWFAPPQTDWPRRVALTGFPLFDEGDFRDVDEELEEFLAGHKAPVVFTPGSTMVDGMSFFKAATDALRSLGQRGIFLAKQGEQMPPLPPSILVRSYVPLSKLLPRAKALVHHGGIGTVSQALAAGIPQLAVPFAHDQFDNAERMKRLGCGLRLDAPVTGPSLLKSLRKLLEEESFRESCAETQLLVDPGEISCLKALSIVEAVGATTIRPARSKLGPQLVRAS

SEQ ID NO: 10 - protein, 441 aa, synthetic construct
MAKVIITAIGSAGDVHPLLGIGAALAARGHEIVFCTHPPFAEAASRQGFAFVPIGTAAEYEAAMANPALWHPRTSFRTLWAVIAPTLRPHFDTLAALTDSDTVLVGTLWAFSARLMQELYRVPFVSVQVSPSTLLSAHAPPTHPRLTIPRGLPLALKTALLRLIERHVLDKICGPALNALRVELGLAPARRIFGEWLHSTDGVLCLFPDWFAQAQPDWPSPRCLSGFPLFNDTGVQAPDPQLEAFLAAGERPVVFTAGSTLIDQARYTDAVSAALAESGLRGILLTPDAPAAQTDAQTDAQGMLLKRRYVPLQKLLPRCRALVHHGGIGTAALAYAAGIPQVVTPFAHDQFDNAQRVVVSGCGVRLDKPLEPQALARALKHVLGSPSIATQCGRVQDRLASSLDGCEAAARYIEGFVPAGVRAQTVGRASIPALADSGHSP

SEQ ID NO: 11 - protein, 423 aa, synthetic construct
MSQIVITAIGSAGDVHPLLGIGRALARRGHRVVFCSHPPFEELALKEGFRFVPIGTRAEYESAINNPALWHPRTSFRTLWNVIAPSIRPHYDALCTLVDDDTVLVGTLWAFAARLMQELHQVPYISVQISPSTLLSAIAPPTHKRMNLPTWLPLPVKKQCLAFLEKKMLDKTCGPALNQVRASLGLPPATRILGRWMHSTDSVLCLFPAWFATPQADWPTNHWMSGFPLFDDVAAQRLDGEVTGFLATGNAPVVFTPGSTGIDAHTYFGAVDKAIRNTGMRAIVLARNTGDRWRDRPDVLVREFVPMSALLPRCAGIVHHGGIGTAALAYAAGVPQIVTPFAHDQFDNAQRIVKSGCGVRLDSPIDGAVLTEALKSVIRNPEMASRCAEAAQRVLQAGDCCDAAAQHIEGFASRAARVEKKAA

SEQ ID NO: 12 - protein, 430 aa, synthetic construct
MHILIIAIGSAGDVHPFLGLGRTFARQGHRVSFCTSPAFAPIVERSGLRLLPLGTAEEYQAAMNDPALWNPKTSFKVLWKIMAGFIRPLFDILETEVDNETIMVGSLWAFGARLMQEKYGVPYVSVQVSPSTFISARLPPVHKRFSVPASWPYSVRAALFWAIERGILDRVCGPDLNRERATLGLPPVKHILGQWLHSPQGVLGLFPRWFAPPQIDWPRQVSLTGFPLFDESEFHEMDAELEDFLAGGPAPVVFTPGSTMVDSMTFFKTAADTLNGLGKRGIFLAKQGEPMPPLPSSIVVRSYVPLSKLLPRAKILVHHGGIGTVSQAFAAGIPQLAVPFAHDQFDNAARMERLGCGLRLDAPISGPSLRTALLRLLEEESFQKNSTATRLRVDSGEVSCLKALSVIEAVGVSHLHVAGPSSGPQLVHAG

SEQ ID NO: 13 - protein, 435 aa, synthetic construct
MHVIITAVGSAGDVHPFVGIGRTLVARGHRVTFCASAAFAPLIERCGFNFLPLGTREEYDEVMADPALWHPRTSLPTLWKTIASTLREQYRLLEQACDDETVMLGSLWAFSARLLQEKHGIPLVTAQVSPFGFWSAAAPSTHPPGANLPAFVPYPVKRLLLGMIERAFLDRVMAPELNSFRQELGLAPTKRIISQWISSPDRVLGLFPEWFAPVQHDWPAQTVLTGFPLFDESGLQPLDAELEDFLNEASPVVFTPGSTMVNGEQYFTAAVQALKLINRRGVFLTSQSVDPSVSSEGRILVRRYVPMSRILPHSAALVHHGGVGTTALAMAAGVPQIATPFAHDQFDNAARMERLGCGLRVFPPASAESLASALDKILNSQEVRASCERYRTLIPSGDSASEAAALQIEALARTARSRAAENLYFQGDYKDDDDK

SEQ ID NO: 14 - protein, 436 aa, synthetic construct
MNVFLATLGSSGDVFPFILIGEHMAKRGHKVFLCTNPYFKGLAESRGLNFISVGSTQEYIAGVHSRALWDKKTALDTLCRYMLNQQEEMYQQLERVIDSSSVLLTSLWSFSAKALGEKKGCCTLPVRVGPSTFLSCYNLPQHGKLALANKLPMPLRKGMVSLFEKKVLDKRLCAEVNAQRSRVGLPPIKRILSQWVHSSADGLICLFPEWFVHPQPDWPRNANLVGLPLFEAQDAPADEELARFLESETVMFSPGWSLQFDEYFFNAVIAEVTRQRRQCLVVGVNPDDVPVRPGVLVRKEVDLESCLRHCVAAVHHGGIGTISHCFYQSVPQLIFPSAFDQFDNAYRVEQLGCGIQAKSRLPEKFSDDLTRVMTDENMRKQCRRIQEKYGEKNHCLENVRHALEQAHQRHAPTPQCRLQMAENLYFQGDYKDDDDK

SEQ ID NO: 15 - protein, 427 aa, synthetic construct
MNLFLATLGSSGDVFPFILIGEHMARRGHKVFLCTNPYFKDLAESRGLNFISVGSTQEYISGVHSRALWDKKTALDTLCRYMLKQQDEMYQQLARVVDSSSVLLSSLWSFSAKILGEQKGCCTLPVRVGPSTFLSCYSLPQHGKLALANKLPMTLRKAVVSLFETKVLDKRFCAEVNALRSRVGLAPVKRILSQWVHTSPDGLICLFPEWFAHPQPDWPRNANLVGLPLFEAENAPVDEELGRFLERETVLFSPGWSLQFDEYFINAVIAEVTRQGRQCLVVGVNPEDVPVRPGVLVKKEVDLTACLRHCVAAVHHGGIGTLSHCFYQSIPQLIFPSAFDQFDNAHRVEQLGCGIQAKSRLPETFSDDLARVMTDENMRERCRRIQEKYGDKNHCLDNVRHALEQAHQRHASENLYFQGDYKDDDDK

SEQ ID NO: 16 - protein, 435 aa, synthetic construct
MHVIITAVGSAGDVHPFVGIGRTLVARGHRVTFCASAAFAPLIERCGFNFLPLGTREEYDEVMADPALWHPRTSLPTLWKTIASTLREQYRLLEQACDDETVMLGSLWAFSARLLQEKHGVPLITAQVSPFGFWSAAAPSTHPPGANLPAFVPYPVKRLLLGMIERAFLDRVMAPELNSFRRELGLAPTKRIISQWISSPDRVLGLFPEWFAPVQHDWPAQTVLTGFPLFDESGLQPLDAELEDFLNEASPVVFTPGSTMVNGEQYFTAAVQALKLVNRRGVFLTSQSVGPSVSGEGRILVRRYVPMSRILPHSAALVHHGGVGTTALAMAAGVPQIATPFAHDQFDNAARMERLGCGMRVFPPASAESLAAALDKVLNSQEVRASCDRYRTLIPSGDSASEAAALQIEALARSARSRAAENLYFQGDYKDDDDK

SEQ ID NO: 17 - protein, 421 aa, synthetic construct
MHVIITAIGSAGDVHPFIGIGRTLAARGHRITFCASAAFAPVIERCGFKFLPLGTREEYQAVMADPALWHPRTSLPTLWKTVGGTLHEQYRLLEQACDDDTVLLGSLWAFSARLLQERHGVPLVTGQVTPFAIWSPLAQPVQPPGTNLPAFVPYPLRRLLLGVIEHTFLDRLMKPELNGFRRELGLPPVKRIISQWISSPDRVLGLFADWFAPIQQDWPAQTVLTGFPLFDESGFEAPDTELEDFLSVAPPVVFTPGSTTVNAEQYFAAAAKALKLIDRRGVFLTRQPVDPSLSSDGRILVRPYVPMSRILPHTSTLVHHGGVGTTALAIAAGVPQITTPFAHDHFDNAARMQRLGCGLQVSTPASAESLATALDTVLNSPDVRASCDRYSKLMASGDSAREAAALQVEAIARRAAIYHVA

SEQ ID NO: 18 - DNA, 1263 bp, synthetic construct
  source 1..1263: mol_type = other DNA
atgcatgtgatcattacggccgtcggttcggccggtgacgttcatccatttgtcggcatcggccgtacgctcgtggcacgcgggcatcgggtcacgttttgcgccagtgcggcgttcgcgccgttgatcgagcgctgtggtttcaatttcttgccattgggtacccgtgaggaatatgacgaggtcatggccgatccggcgctgtggcatccgcgcacgtcgctgccgaccctttggaaaaccatcgccagcaccctgcgcgaacagtaccggctgctggagcaggcctgtgacgacgaaacggtcatgctcgggtccttgtgggcattctccgcgcgtctgttgcaggagaaacacggtatcccgctggtcaccgctcaggtgtcgccgttcggtttctggtcggccgccgcgccctcgacccacccaccaggagccaatctgccggccttcgtaccctatccggtgaagcggctgttgctggggatgatcgaacgggcatttctcgaccgggtcatggcaccggagctcaacagtttccgccaggagctgggtctcgcgccgaccaagcgcatcatcagccaatggatttcctcccctgaccgggtgctggggttgtttccggagtggttcgcccccgtgcaacacgactggcctgcccagaccgtgctgaccggcttcccattgttcgatgagtccgggctgcaaccgctcgacgccgaactggaagacttcctgaacgaggcatcgccggtggtgttcaccccaggctcgaccatggtcaatggcgagcaatatttcactgcggcggtgcaagcactgaaattgatcaaccgccgcggcgtgttcctgaccagccagtcggtagacccttcagtgtccagcgaaggcaggatcctggtacggcgctacgtgccgatgagtcgcatcctgccccacagcgcggcactggtgcatcacggcggcgtcgggaccacggccctggccatggccgccggcgtgccgcagatcgccacgccgttcgctcacgaccagttcgacaacgccgcgcgaatggagcgtctgggctgtggcttgcgggtcttcccgcccgcctctgccgagtcgctggccagcgcgctggacaagatcttgaacagccaggaagtacgcgccagttgcgaacgctatcgcacgttgattccgagcggtgacagcgccagtgaagcggcggccctgcagatcgaggcgctcgcccggactgccaggtcacgcgctgcctaa

SEQ ID NO: 19 - DNA, 1266 bp, synthetic construct
  source 1..1266: mol_type = other DNA
atgaatgtgtttttggcgacccttggatcgtcgggtgatgtatttccttttattttgattggcgagcacatggccaaacgtggccataaggtatttctgtgcaccaacccttatttcaaaggtttggccgagtctcgcgggttgaattttattagcgtaggctcaacccaggaatatatcgcaggggtgcattcaagagcgctgtgggacaaaaaaacggccttagataccctgtgccgctacatgctgaaccagcaggaggagatgtatcagcagttggagagggtcatcgatagctcttcggtgctgctcacgtcgctctggtctttttccgccaaagcgttaggggagaagaaaggctgctgtacgctcccggtacgcgtggggccttcgacgtttttgtcgtgctacaacctgccgcagcatggaaaactggcgctggcgaacaagctcccgatgccgctgcgtaaaggcatggtctccctgttcgaaaaaaaggtgttggataaacggctctgcgcagaggtcaacgcgcagagatcgcgcgtgggcctgccaccgatcaaacggattctgtcgcagtgggttcactcgtccgcggacgggctcatttgtctgtttcccgagtggttcgtccatccgcagccagactggccgcgcaatgccaacctcgtgggactgccgttgtttgaagcgcaagacgcgcccgccgacgaagagctggcccgttttctggaaagtgaaacggtgatgttctctccgggctggtcgctgcagttcgacgaatacttcttcaatgccgttatcgccgaagtgacgcggcagcggcggcaatgtctggtcgtgggggtgaatccggacgatgtccctgtgaggccgggggttttagtcagaaaagaggtcgatctggagtcctgccttcgtcactgcgtggccgcggtacaccacggcggtattggcaccatctcgcactgcttctatcagagcgttccgcagctgattttccccagcgcgttcgatcagtttgataatgcctaccgggtcgaacaactgggctgcgggattcaggccaaaagccgtctgccggagaaattttctgacgatttaacccgcgtgatgacagacgaaaacatgcgtaagcaatgccgccgtattcaggagaaatacggcgaaaagaatcactgtctggagaatgtcaggcatgcgctggagcaggcgcatcaacgccatgctccaacgccgcagtgtcgcttacagatggcctga

SEQ ID NO: 20 - DNA, 1239 bp, synthetic construct
  source 1..1239: mol_type = other DNA
atgaatttgtttttggcgacccttggatcgtcgggcgatgtatttccctttatcttgattggcgagcacatggccagacgtggtcataaggtattcctgtgtaccaacccttattttaaagatttggctgagtctcgcgggttaaattttattagcgtaggttcaactcaggaatatatctctggggtgcattcaagggcgctgtgggacaaaaaaacggccttggacaccctgtgccgctacatgctgaaacagcaggatgagatgtatcagcagttggcgcgcgttgtcgacagttcctccgtactactctcgtcgctctggtctttttccgccaaaatactgggagagcagaaaggatgctgcactcttccggtacgcgtggggccatcgacgtttttgtcgtgctacagcctgccgcagcacggaaaactggcgctggcgaataagcttccgatgacgctgcgcaaagcggtggtctcactatttgaaacaaaggtgctcgataaacggttctgcgccgaggtcaacgcgctcagatcccgagtgggcctggcccccgtcaaacggatcctgtcgcagtgggttcatacgtctcccgacggtctgatctgtctttttcccgagtggttcgcccatccacagccagattggccgcgcaatgccaaccttgtggggctgcccttgtttgaagcggagaatgcccccgtcgacgaagagctaggccgctttcttgaacgtgaaacggtgttgttttctcccggctggtcgctacagtttgatgaatatttcattaatgccgttatcgccgaagtcacacgccaggggcggcagtgtctggtcgtaggggtgaatccggaggatgtccccgtgcggccgggcgttttagtcaaaaaggaggtcgatctgacggcctgcctgcgtcactgcgtcgcggcggtacatcatggcggcatcggcaccctctcgcactgtttttaccaaagcattccgcagctgattttccccagcgcgttcgatcagtttgacaacgcccaccgggtggaacagttaggttgtggcattcaggccaaaagccgcctaccggagacgttttctgacgatctcgctcgggtgatgaccgatgaaaacatgcgtgagcgatgccgccgtattcaggagaaatacggcgacaaaaatcactgtctggataatgtcaggcatgcgctggagcaggcgcaccagcgccatgcctcttga

SEQ ID NO: 21 - DNA, 1263 bp, synthetic construct
  source 1..1263: mol_type = other DNA
atgcatgtgatcattacggccgtcggctcagccggtgacgttcatccatttgtcggcatcggccgcacgctggtggcacgcgggcaccgggtcacgttttgcgccagcgcggcgttcgcgccgttgatcgagcgctgtggtttcaatttcttgccattgggtacccgtgaggaatatgacgaggtcatggctgacccggcgctgtggcatccgcgcacgtcactgccgaccctctggaaaaccatcgccagcaccctgcgtgaacagtaccggctgctggaacaggcctgtgacgacgagacagtcatgctgggttccctgtgggcattctcggcgcgtctgctgcaggaaaagcacggtgtcccgctgatcaccgcacaggtgtcgccgttcggtttctggtcggccgcagcgccctcgacccatccaccgggcgccaacctgccggccttcgtgccttatcccgtgaagcggctgctgctgggaatgatcgaacgggcatttctcgaccgagtcatggcgcctgagctcaacagcttccgccgcgaactggggctcgcgccgaccaaacgcatcatcagccaatggatttcctcgcctgaccgggtgctggggttgtttccggagtggtttgccccggtgcaacatgactggcctgcccagaccgtcctgaccggcttcccattgttcgacgagtcgggtctgcaaccgctcgacgccgaactggaagacttcctgaacgaggcatctccagtggtgttcaccccgggctcgaccatggtcaacggcgagcaatatttcaccgcggcggttcaggcgctgaagctggtcaaccgccgcggcgtgttcctgaccagccagtcggtgggtccttcagtatccggtgaaggcagaatcctggtacggcgctacgtgccgatgagtcgcatcctgccccacagcgcggcgctggtgcatcacggcggtgtcgggaccacggccctggccatggccgccggcgtgccgcagatcgccacgccattcgctcacgaccagttcgacaatgctgcacgaatggaacgtctgggctgtggcatgcgggtcttcccgcccgcctccgccgagtcgctggccgcagcgctggacaaggtcttgaacagccaggaagtacgcgccagttgcgaccgctaccgcacgttgattccgagcggtgacagcgccagtgaagcggcagccctgcagatcgaggcgctcgcccggtctgccaggtcacgcgctgcctga

SEQ ID NO: 22 - DNA, 1263 bp, synthetic construct
  source 1..1263: mol_type = other DNA
atgaatttgtttttggcgacccttggatcgtccggcgatgtatttccttttattataattggtgagcacatggccaaacgtggccataaggtctttttgtgtactaacccttattttaaatgtttggccgagtctcgcgggctggaatttatcagcgtgggttcagcgcaggaatatattgctggggtacattcaagagcgctgtgggacagtgaaacggcattggatactctgtgccgctatatgttgaaacagcaggatgagatgtaccagcagttggagcgcgttgtagacggctcctcggtgctgctgacgtcgctctggtctttttccgccaaaacgttgggagagcggaaaggctgctgcactcttccggtacgtgtggggccatcgacgtttttgtcgtgctacaacctgcctcagcatggaaaactggcgctggcgaacacgcttccgatgacgctgcgcaaaggaatagtgtcactgtttgaaaaacaggtgctcgataaacggttctgcgccgaggtcaacacgctgagatcgcgcgtgggtctggcgcccgtcaaacggatcctgtcgcagtgggtgcatacgtctgccgatggtttgatctgcctgtttcccgagtggttcgcccatccgcagccagactggccgcgcaatgccaaccttgtggggctgcccttgtttgaagctgagaatgcgcccgtcgacgaagaactgggccgcttccttgaacgtgaaacggtgttgttctctcccggctggtcgctgcagttcgatgaatatttctttaatgccattgtcgccgaagtcacacgtcagcggcggcagtgtctggtcgtcggggtgaatccggaggatgtccccgtacggccgggcgttttagtcaaaaaggaggtcgatctggcggcctgcctgcgtcactgcgtggctgcggtacaccacggcggcatcggcactatctcgcactgcttctaccaaagtgtgccacagttgattttccctagcgcgttcgatcaatttgataatgcttaccgggttgaacagctaggttgcggcattcaggccaagagccgcctgccggagaaattttcagacgccctcgatcgggtgatgaccgatgaaaacatgcgtgagcgatgccgccgtattcaggagaaatacggcgacaaaaatcactgtttggagaatgtcaggcacgcgctggagcaggcgcaccagcgccatgcctcctgccccggcagcgtcgcttacagatga

SEQ ID NO: 23 - DNA, 1353 bp, synthetic construct
  source 1..1353: mol_type = other DNA
atgaatattctgctcaacccagcggggtcatcgggtgatgtcctgccttttattcgtttaggaatggcactgaaagcacggggccatgacgtcactgtaattacgatgggccgctatgaaagcctcgtccgccatgccaatcttgccttcgtggaatggatctcgccggaagccatgcaggcaatcgaagacaacccggatctttggcacccaacacgtggattacttttggccattcggacactggaaccttggtacgcccagttgcatgagcatatacaagcgcgatatgttccgggtcagacagtactggtcgcaggaagcctgaacttagctgctcgacttgctcacgatcgtttaggcgttcctctggcaaccttgctgttgcagccggcttatcttcggagtgtgcatagatctccggttttacctggtgttcctactccggattgggcgccacgaatatggaagcgtctggtgtattttctgagcgatgcattagtacttgactgggtggcggctcccgctgtcaatgcgctgcgtgctgaacttggattaccgcccgcgcgcggggtcctgggttcctggtggctgtcacctcaacgggtgatcggcctcttccccgattggtttggggagccccagcctgattggccagcgcaggtacgcttaactgatttccctctgtacgacggtttaggagcagaaccagttccggcgggcttagacgagtttctggaaaatggcgaaccgccagtggtgtttaccccgggcaccatgatgcgtcatccccgtcgtttttttgccgaggccgtagacgcgtgcggccgccttggtcggcgcggtctgctcttgacgcgctatcgagaccagttaccagctcgcttgccttccggggtttgtcatttcgattacgtgccattaagtcatgttctgccacgcgccgcagcgttagtgagtcacgggggtatcggtaccgtatcccaggcactggtggcggggacgccgcagctcattatgccgttgggttttgaccagttcgataatgccgcaagattagaacacatgggcgttgcaactaccctggcgccccgtgcatttcgtgggagagcggttgcccaacgggttgcacgtctgctggattcagccacagttgcccgtgcatgtaaagcagccgcagtgcgcctgcataaagatagatgggtcggacccgcttgccaagctgtagaggaactcgcgggtgaggatggagccgcgcaattgccgggctcttctggttgctttccggctagacaagctagccaccctgatccgaaagctgttgaagattatcttattgcctgtgccctgcca

SEQ ID NO: 24 - DNA, 1281 bp, synthetic construct
  source 1..1281: mol_type = other DNA
atgcaaatccttatgatagctattggaagcgctggagatgttcaccccctgctcgccgtgggccgtgaattagctaatcgtggcaatcgtgtgactgtgtttacgaaccctgcttttcagccattggttgaacgatgcgggctgggcttttatgcactgggcactgcgcaggagtaccaccgggcaatcgagaccccagcattatggaatcctagcacctcattcgctacattatggcggattgtcagcccagtgctgagacctatggttgagggtttactggcgactattgacgatgagacggtcatggttggtagcctgtgggccttcggagcacgtatagttaacgaactctacgacgtccctcttgttaccgcccaggtttcagccagtacatttctctccgctcagcttccgcctgttcataaacgggtaagaataccggcaggtctgccctatggggtgaaaagtggtctgctgtggcttattgaacgcgcggcgctggaccgcgtatgtgcgccggcgttaaacgagctgcgtggggaactgggcttagcccctgtatcacgagttcttggtcgctggatgcatagtccacattgtactctggggttgtttccggattggttcgcaccgccgcagcacgactggcccctgcatgtcacattgacgggtttcccgctgtttgacgaaccggaacgttcagtcgcggatcccgaattgggtgaatttttggaagccggttccccgccggtggtgttcacgcaaggttcgacctctgtttctggaccactctatgtggagacggcgttgaaaactttaaaacaccttggccttcgtggtctttttttgggcggagatgcaggccaattaccacagggcgatggcatcctgcaccgtagatttgcaccgctcgcctcggtattaccaaagagtgcagctatggtccatcatggtggcattggaacaatgagctttgcttttgcggctgggattccgcaactggccacccctttcgcacatgatcaatttgacaatgcagcacgcctcgcacgcctgggttgcggggtccgccgtgattggccggtggattcctcttctatggcttcgaccctgcaggctctgctggaatctcctgaggttagagatgcgtgccggacagtacgtcgcagaatgaagccagcccatgtagcccgggcggatgccgcggggatgatcgaacgatgtggattagcggcccgcccccgcgtattagcagtgtcccaggccgaacataatgtt

SEQ ID NO: 25 - DNA, 1281 bp, synthetic construct
  source 1..1281: mol_type = other DNA
atgcacgccattctcatcgccatcggttcggccggcgacgtgttccccttcatcggcctggcccgcaccctgaagttgcgcggccaccgcgtcagtctgtgcagcattccggtgtttcgcgccgcggtggagcagcacggcatcgagttcgtcccgctcagcgacgaactgacctaccgccggaccatgggcgacccgcgcctgtgggatccgaagacctcgttcggagtgctctggcaggccatcgccgggatgatcgagccggtctacgagtacgtctgcgcacagcgccacgacgacatcgtggtggtcggttcgctgtgggccctgggcgcgcggatcgcccaagagaaatacgggattccctacctgtcggtgcaggtctcgccgtcgaccctgctgtcggcgcacctgccgccggtccaccccaggttcaacgtgcccgagcaggtcccgctggcgatgcgcaagttgctctggcgctgcatcgaacgcttcaagctggaccgcacctgcgccccggagatcaacgcggtgcgccgcaaggccggcctggtcggcccggcgaagcgcatcttcacccagtggatgcattcgccacagggagtgctctgcctgttcccggcctggttcgcaccgccccagcaggactggccgcaaccgctgcacatgaccggcttcccgctgttcgacggcagcgtcccggggacccgcctcgacgacgagttgcagcgcttcctcgagcagggcagtcggccgctggtgttcacccaggggtcgaccgagcacctgcagggagacttctatgccatggccttgcgcgcgctggagcgtctcggcgcccgcggcatcttcctcaccggcgccggccaggagccgctgcgtggcttgccgagccacgtgctgcaacgctcgtacgtgccgttgggggccttgctgccggcgtgcgccgggctggtccacccggccggcatcggcgccatgagcctggcgctggcggcgggggtgccgcaggtgctgctgccttgcgcccacgaccagttcgacaatgccgaacgcctggtccgcctcggctgtggtatccgcctgggcctgccgctacgcgagcaggcgctgcgcgagtcgctctggcggctgctcgaggacccggcgctggcggcggcctgtcggcgtttcatggaattgtcacaaccgcacagtatcgcttgcggtaaagcggcccaggtggtcgaacgttgtcatagggagggggatgtgcgatggctgaaagccgcgtcctga

SEQ ID NO: 26 - DNA, 1290 bp, synthetic construct
  source 1..1290: mol_type = other DNA
atgcatatcctgataatagctattggctcagctggcgacgtacatccatttctgggattagcacggacctttgttaagcagggtcatcgagtctctttttgcacgagccccgccttcgctccagttgtggaacgctgtgggctgagaatgctgccgctgggtactgcggccgagtatcaggcagccatgaatgatcctgcgctctgggaccctcggacaagttttaaggtcttgtggaaaattatggccggatacatccgcccgctttttgacatcctcgaagctgaaattgataacgaaacggtgctggtcggctccttgtgggcgtttggggcaagactgatgcaggagaaatacggcgtgccctatgttagcgtgcaggtcagccccagcaccttcctgtccgctaagcttccgcctgtgcataaacgtttcacaattccttcatttttaccatcttctttgagagcagctttattgcacacaatcgaacggcacgtcattgaccctgtgtgcgcgccagatcttaatcgtgtgcgcgataatcttggactcccgccggtacaatctatactgggacaatgggttcatagtccgcagggggtcttaggtttatttccagcatggttcgcgccgccacagacggattggcctcgtcgtgtagccctcactggcttcccactctttgatgagggcgattttcgcgacgtagacgaagaattggaagagtttttggcaggccacaaggccccggttgttttcacccctggtagtactatggtagatggcatgtcgttctttaaagccgcgaccgatgcgttacgtagtctgggtcagcgtggcatttttctcgccaaacaaggtgagcagatgccgccactgccgcccagtattctggttcgttcctatgtcccgctgtctaaacttctgccgcgggcgaaagcactggtacatcacgggggtatagggactgtgtcacaagcactggccgctggaatcccgcagttagcagtgccctttgcgcatgaccagttcgataacgccgaacgcatgaaacgacttggttgtggtttacgcttggatgcgccggttacgggaccttccttactgaagtcgcttcgcaaacttctggaagaggagtcttttcgtgaatcgtgtgctgaaacccaattactggttgatccaggtgagattagctgcctgaaagcgttgtcaatcgttgaagctgtgggggcaacaaccattcgtcctgccagatcaaaacttggtccccaactggttcgagcaagc

SEQ ID NO: 27 - DNA, 1326 bp, synthetic construct
  source 1..1326: mol_type = other DNA
atggcgaaagtcatcatcaccgcgatcggctcggcgggcgacgtgcatcctttgctcggcatcggcgcggcattggccgcgcgcggtcacgagatcgtcttctgcacgcatccgccgttcgcggaggccgcctcgcgccagggtttcgcattcgtaccgatcggcaccgccgccgaatatgaagcggcgatggctaacccggcgctatggcatccgcgtacgtcattccgcacgctatgggcggtgatagcgcctacgctgcgtccgcatttcgatacgctggctgcgttgaccgatagcgacaccgtgctggtcggcacgttgtgggctttttccgcgcggttgatgcaggagctgtatcgggtgccgttcgtatcggtgcaggtctcgccgtcgacgctgctctccgcgcatgcgccgcctacccatccacggttgaccattccgcgcggcttgccattggctctaaaaacggcgctgctgcgactgatcgagcgccacgtactcgacaagatctgcggccccgcattgaacgcgttgcgcgtggaactggggctcgcaccggcgcgccgcattttcggcgagtggctgcattcgaccgatggtgtgctgtgtctctttcctgactggttcgcgcaagctcagccggattggccgtcgccgcgttgcctcagtggttttccgctgttcaacgacacgggcgtgcaggcgcccgatccccaactcgaagcctttctcgccgcgggcgagcggccggtggttttcaccgcgggttcgaccttgatcgatcaggcgcgctacacggatgcagtgagcgcggcgctggcggaaagcgggctgcgcgggattcttttgacgccggatgcgcccgccgcccaaacagatgcgcagacagacgcacagggcatgctgctcaagcggcgttacgtaccgctgcaaaagctgttgccgcgctgccgggcgctggtgcatcacggcggcatcggcaccgcggcgctcgcgtatgctgccggcattccgcaagtggtgacgccgttcgcgcacgatcagttcgataacgcgcagcgtgtcgtcgtgagcggttgcggcgttcggctcgataagccactggagccgcaggcgcttgcgcgtgcgttgaagcatgtgctcggcagcccgtcgatcgcgacgcagtgcggacgcgtgcaggaccggcttgcgtcgtcgctggatggctgcgaggcagccgcacggtatatcgaaggtttcgtgcccgccggcgtgcgcgcgcaaactgtcggccgcgcgtctatcccggcactcgcagacagcgggcatagcccatga

SEQ ID NO: 28 - DNA, 1272 bp, synthetic construct
  source 1..1272: mol_type = other DNA
atgtcccagatcgtcattacggcaatcggttcggcaggcgacgtccatccgctgcttggcatcggccgtgcattggcacgccgcggccaccgcgtggtcttttgttcgcatccgccgttcgaggaattggcattaaaagaagggttccgttttgtcccgatcggcacgcgcgccgagtacgaaagcgcgatcaacaatcctgcgttgtggcatccgcgcacctcgtttcgtacgttatggaatgtgatcgcaccatccattcgtccacattacgatgcattgtgcacactcgtcgatgacgacaccgtattggtgggaacgttgtgggcattcgccgcccgcttgatgcaagagctccatcaggtaccgtacatctcggtacagatctcgccatcgacgttgttgtcggcgatcgcaccaccaacgcacaagcggatgaacctgcctacgtggttgcctttgcctgtgaagaaacaatgcttggctttcctcgaaaaaaagatgctcgacaagacatgcggacctgcattgaatcaggttcgcgcgtctcttggcttaccccctgcgacgcgcattctggggcggtggatgcattcgaccgacagcgttctctgcctgtttcccgcttggtttgcgacacctcaggcggattggccaacgaatcactggatgtctggttttccgctatttgacgacgttgcggcgcagcggctcgacggcgaggtgacgggatttcttgccaccggcaatgcgcctgttgtatttactccgggatcaaccggtatcgacgcgcatacgtatttcggtgctgttgataaagcgatccgaaacaccggtatgcgcgcgatcgtactcgctcgtaacacgggcgatcggtggcgtgatcgtccggacgttctggtgcgggagttcgtgcccatgagcgcgttgttgccacgctgtgccgggattgttcaccacggcggcatcggaaccgcagcattggcgtatgcggcgggcgttccacaaatcgtgacaccgttcgcccatgaccaattcgacaatgcgcaacgtatcgtaaaaagtggctgcggcgtgcgtcttgacagccctatcgatggcgctgtattgacggaggcgctgaagtcggtgatacgcaatcccgagatggcatcccgttgcgcggaagccgcgcaacgtgtgctgcaagcgggcgactgctgcgatgccgccgcccaacatatcgaagggtttgcaagtcgcgcggccagggtggagaaaaaagcggcctga

SEQ ID NO: 29 - DNA, 1293 bp, synthetic construct
  source 1..1293: mol_type = other DNA
ttgcatattctcatcattgccatcggctcagcgggtgacgtccatccctttctgggattgggccgtacctttgcgaggcaggggcatcgcgtctcgttctgcaccagtccagcctttgcaccgatcgttgagcgctccggcctgaggcttctacctctcggcaccgctgaagagtaccaagccgccatgaacgaccccgcgctgtggaatccaaagacctccttcaaggtgctctggaagatcatggctgggttcatccgccctctcttcgacatcctcgagactgaggttgacaacgaaacaatcatggtcggttcgttgtgggcgttcggtgcccggttgatgcaggaaaagtatggcgttccctatgtctctgtccaggtctcgccctcaacattcatctctgccaggctgccaccggtccataaacgcttctcggtcccggcttcgtggccgtactcggtacgtgccgctctgttctgggcgatcgagcgcggcatcctcgaccgcgtctgcggaccagacttgaaccgtgaacgtgccactctcggactcccgcccgtaaaacacatcctcggccagtggttgcactcccctcagggagtacttgggctcttcccgcgctggttcgctccaccccagatcgattggccccgacaagtctcgttgacaggatttcctctcttcgacgaatccgagtttcatgagatggatgctgagctggaagattttctagcaggcgggccagcccctgtcgtcttcacgccggggtccaccatggtggatagtatgaccttcttcaagacagcggcggacacgctgaacgggctcgggaagcggggaatctttctcgccaagcaaggcgaaccgatgccacctcttccctccagtattgtcgtccgctcctatgttcctctcagcaaacttctgccccgtgccaaaattctggtgcaccatggcggcattgggacagtatcccaggcgtttgcggcaggtatcccccagctcgccgtaccgttcgcccacgatcagttcgacaacgctgcccggatggagcggttgggctgcgggctgcggctcgacgctcccatcagtgggccttccctgcgaaccgcgctcctgcggctgctcgaagaggagagctttcagaagaacagcacagcgactcggctgcgcgtcgactcaggtgaggtgtcctgtctgaaggctttgtcggttattgaagcggtcggggtctcgcacctacacgttgccgggcccagctcaggaccgcagctcgtccatgcaggatga

SEQ ID NO: 30 - protein, 481 aa, synthetic construct
MISFPKDFRFGWSQAGFQSEMGSGDSDPNSDWFKWVHDQENIAAGLVSGDFPEDGPAYWVNYRTFHDNAERMGLTMARIGVEWSRLFPNPPPRVKFTERDGKVLEVEVGDLKTLDQYVNKTALEHYREMFKDIKSRGIHFVLNFYHWPLPLWVHDPVRVRKGDLTGPTGWLSTTTVAEFARFAAYVAWKFEDIADEYSTMNEPNVVWGLGYTSVKSGFPPGHLSFELTERAFRNIVQAHCRAYDAAKSVTRKPVGIIYANSSFEPLTEKDKEAAEQAEHDNRWKFFDAVIRGKLGEEVRDDLKGRLDWIGVNYYTRTVVKAVEKGYRGVGGYGHGCERNSLSSAGRPTSDFGWEFYPEGLHDVLVKYWRRYGLKMYVTENGIADEADYQRPYYLVTHLSSVRRAIDDGADVRGYLHWSLADNYEWASGFTPRFGLIQVDYSTKRQYWKPSALVYREVAKSSGVPDDLEHLNRVPPVKPLRH

SEQ ID NO: 31 - protein, 485 aa, synthetic construct
MSTFKPGFKFGWSQAGFQSEMGLPGGEDPNSDWYVWVHDRENIIAGMVSGDLPENGPAYWHNYKKFHDIAEDMGLKIARIGVEWSRIFPNPTFDLHVDAEVKEGKVTYVDVKQSDLEKMESIANKEAVEHYRKMFSDLKERGIEFILNLYHWPLPLWIHDPIAVRRGEKTERTGWLSTKTVLEFSKFSSYISWKLDDLVDAYSTMNEPNVVWGAGYSGVKSGFPPGYLNLSYMGVAMFNIIQAHARAYDLLKVHKKPVGIIYANSDFQGLTQEDEEAASRAEYDNRWAFFDAIINGKYKDEVREDLKGKLDWIGVNYYTRSVVRKSGDGYTGVPGYGHGCERNSLSAGGRPTSDFGWEFYPEGLRNVIVKYWKRYGLPIYVTENGIADEGDYQRPYYLVSHIYQVYKAIEEGADVRGYLHWSLADNYEWASGFSQKFGLLRVDYSTKRLYWNPSALVYREIAKKGGIPESVEHLNRIPPIKGLRR

SEQ ID NO: 32 - protein, 483 aa, synthetic construct
MISLPGIRFGWSQAGFQSEMGLPGSEDPNSDWFAWVHDKENIAAGVVSGDLPEYGPAYWHRFREFHDAAERMELKIARIGVEWSRVFPKPTLDVQVDIEQRGDMVTHVDVSQSQLEKMDAIASKDAVEHYRTIFSDLKRRGIEFVLNLYHWPLPLWIHDPVAVRRGEKTERTGWLSTRTVVEFAKFAAYISWKLDDLVDAYSTMNEPNVVWGAGYTSVKSGFPPGYLSFAHSSRAMYNMVQAHARAFDVLKTHKKPVGIIYANSDFQGLTAGDADVASKAEFDNRWRFFEAIVNGELEGYRDDLKGRLEWIGVNYYTRSVVRKAGEGYVVVRGYGHACERNSLSADGRPTSDFGWEFYPEGLGNVLVKYWEKYGLPLYVTENGIADEADYQRPYYLVSHIYQVYQALRRGADVKGYLHWSLADNYEWASGFTPRFGLLRVDYTNKSLFWNPSAFVYKEIAGSNGIPDQLEHLNRVPPTRGLRR

SEQ ID NO: 33 - protein, 484 aa, synthetic construct
MGTLFPKDFKFGWSQAGFQSEMGLPGDEDPNSDWFVWVHDRDNITAGLVSGDLPENGPAYWSLYRAFHDDAVKMGLNAARINIEWSRIFPKGMPRPVESNVNMERDEVIHVDIDGHDLEELDGAVNKAAVDRYRNILEDLKSRGIFIVLNMYHWSLPLWIHDPIRVRNGDLRGPSGWIDARTVVDFARFAGYVSWKFDDLVDMYSTMNEPNVVAWSGYIGIKSGFPPGRLDSSLAKKATSNLIRAHARAYDAIKLMTKKPVGIIYANNSYTPLTEKDAAAVELAERDSRWSFLDAVARDGDRDDLRNRLDWIGVNYYSRLMIRSTGNDSYAVVPGYGHVCNGSISRDGRPCSDFGWEIYPEGIYDVLIKYWSRYGIPIYVTENGIADSLDFWRPHYLVSHIHQVHKAMSEGVDVRGYMHWSLADNYEWASGFKMRFGLLQVDYASKRRLWRPSALIYREIAISKAIPDEFEHLTHVPLGKSFNS

SEQ ID NO: 34 - protein, 484 aa, synthetic construct
MSLKFPKDFGFGFSTAGFQHEMGLPGSEYESDWWVWVHDPENIAAGIVSGDLPENGPGYWHLYKSDHDIAFSLGMDTLRLGIEWARVFPKPTFEVNVNADIRDGSVVSVDVSEEALRRLDGLANRDAVQHYIEIIKDWKDRGGKLIVNLYHWPLPLWVHDPLVVRRSGPNNAPTGWLDPRTVVEFAKYAAYLAWRLGEFVDMWSTMNEPNVVFSNGYLYVKSGFPPGYLGIELMLRARGNLMTAHARAYDALREFSKAPIGIIYAISDVQPLTKDDEEAAKAYEEAGQVSFLDAITKGSGREDLRGRLDWLGINYYSRTVVTTAKSQSSILPPARVVPGYGFACGPNAVSRDGRPCSDFGWELYPEGLYNVLTRYWGRYGLPIIVTENGIADARDQWRSWFIVSHLYQLHRALGQGVDVRGYLHWNLIDNYEWASGFRMKFGLVQVDYNTKKRYLRPSALVFREIARNKEIPEYLTHMIQSPTI

SEQ ID NO: 35 - protein, 487 aa, synthetic construct
MRAFPKTFLFGYSWSGFQFEMGLEGSEVPNSDWWVWVHDRENIAAGLVSGDLPENGPAYWHLYKQDHDMAEKFGMNAIRGGIEWARLFPEPTFDVKVEIERDEDGNIVWVDITEKTVEELEKKADMGALEHYRKIYSDWKERGKTFILNLYHWPLPLWLHDPIKVRRFGPDRAPSGWLDDRSVVEFAKFSAFVAYHLDDLVDMWSTMNEPNVVYENGYLRPNSGFPPGYLSFEASKKAKWNMIQAHARAYDAIKTFSEKPVGIIYAYTWLEPLSEDIEEDVRRIRENELYSFVDTVHFGGVNSEERREELKGRVDWLGVNYYSRFAFDKVGGSIVPLPGYGFAGTREGYAKSGRPCSDFGWEIYPEGLEKLLRELSERYRLPMIITENGIADRADKYRSYYLISHLAAVHNAMKAGADVRGYLHWSLTDNYEWAQGFRMRFGLLGVDFSTKRRYVRPSALVFREIATQKAIPDELEHLSDLSVLTKR

SEQ ID NO: 36 - protein, 488 aa, synthetic construct
MSYKFPKDFLFGYSWSGFQFEMGLKGSEVPYSDWWVWVHDEENIVSGLVSGDLPENGPAYWHLYKQDHDIAESLGMNTIRGCIEWARIFPKPTFDVGADVERDEDGNIIHVDVTESAIEKLEKIADMEAVKHYREIFADWKERGKAFILNLYHWPLPLWIHDPIKVRKYGPDRAPSGWLDDRTVIEFAKFAAFISYHFDDLVDMWSTMNEPNVVYQQGYINVKSGFPPGYLSFEGSQKVAKNLIQAHARAYDAIKAYSKKPVGLIYAFAWIEALDEKLEEQVNNIKNQVLYAFVDSVYTGRSPGTEERKDLKGRLDWIGINYYSRHVFGEKDGRLTILDGYGFNCQPGGYCRSGRPASDFGWEIYPEGLGKLIREIASRYNVPLIITENGIADEIDRYRSYYLVSHLYEVYKAIREGLNIKGYLHWSLIDNYEWAQGFSKRFGLVYIDYNTKKRYLRPSAFIYKEIAKNKEIPEELTHLTNIDILVRK

SEQ ID NO: 37 - protein, 456 aa, synthetic construct
MSLKFPKDFKFGFSEAGFQFEMGLPGSENPHSDWWTWVHDQENITAGIVSGDLPENGPGYWHLYQKDHEIADSLGMDSARLGIEWSRLFPKPTFNIKADVEKDSAGNIISVEVGEKSLEELDKIANKEAVEHYRRIFEDWRKRGKLLIINLYHWPMPVWLHDPIKVRKLGPDRAPAGWVDERSVVEFTKFAAYVAWKLGDLPDMWSTMNEPNVVYTQGYVSIKSGFPPGYLSVEASLKAAKHLIEAHARAYDVLKKMTKKPVGIIYATAEIEPLTTEDKEIAEAAYAQHNFSFMDAIFTGTSQLVGGERKDLARHLDWIGINYYSRLVVTRAKTAAGWRVVEGYGFACQPRGISRAGRPCSDFGWEVYPEGLYSVVKRFWERYRLPMLITENGIADSVDALRPRYLVSHLAQVHKLVSEGVELKGYLHWALTDNYEWAQGFRMRFGLVYVDYETKK

SEQ ID NO: 38 - protein, 477 aa, synthetic construct
MNGFPKGFRFGWSQSAFQSEMGNRLNVDSNTDWYKWVHDSSNIVAGRVSGDFPEDGPGYLEHFKDFHREAAFLGLDTARLSIEWSRLFPTGPLPKPVCRTDGDDILSIEIGKEQLQKLDMFVDKNALRRYTEIFRDLRSHDISLILNLYHWSLPIWLHDPLKVRDIGPEATPSGWLSKDIAGQFALYAAYAASKFDDFVLSYSTMNEPNVLYPKFPPGYFGGDISRRVEANLIEAHARAYDAIKSVSEKPVGIISGAPSYVPEKPEDQTAAQQACEERRWPFFDAVKFGRKGSIFRSDLKGRLDWIGLNYYTRCVVHKTNTGFSESDGYGRRCTKNSKSKAGLPTSDFGWEIYPEGLSEVLKSFWNRYSLPLWVSENGVADEKDNLRSYFIVSHLAQVLKSLEEGVDIRGYLHWTLVDNYEWADGFTMKFGLSSFNSKTCQLQRRQSSLVLKDIALAKDIGGRVEGFLNVPEKLVDE

SEQ ID NO: 39 - protein, 485 aa, synthetic construct
MKIPKEFMLGASLSSFQFEGGFRGDEDPNNDWWIWVHDWENIIAGIVSGDFPENGPGYWRLFRQDHDLAEKLGMNTLRVGIEWSRIFPRPTFDVKVTVDKDEDGNILHVDIDEKALAKLDEIADQDAVKHYIEMYSDWKNRGKQLIINLYHWPLPLWIHDPIKVRKYGPDRAPSGWLDEKTIIEFVKYAAYVSWKLRDLADMWSTMNEPNVVYEQGYMFIKNGFPPGYLSFEAAEKAKKNLIYAHARAYEVVKKITGKPVGIIYALPYIESLNGEKETLEAIKSYRIYEFLDLIIKGKSVRNPILRKELASRADWLGVNYYSRIVFKFIHGKPIVLQGYGFFCSSSGVSKMGLPCSDFGWEIYPQGLYLLLKEIHTRYNGLPIIVTENGISDKADKLRPKYLVSHLYNTLKARNEGVPVKGYLHWSLIDNYEWAQGFRQRFGLVIVDFNTKKRYIRPSALVFREIALSQEIPEELMHLTHVEPLI

SEQ ID NO: 40 - protein, 489 aa, synthetic construct
MFPKDFLFGASMAGFQVEMGYAGEDTDPNTDWFVWVREPENLLTGAVSGHLPEYGVGYWNNYPVLHQLAVDFGMNALRTNIEWSRIFPNSTAEVQVIAETQDGITKVEVKKEHLEKLDKLANKKAVEHYRQIFKDMKNRGLKVIVNLSHFTLPLWIHDPISVHKGQQTEKTGWVNDQTVVEFAKFAAYIAWKFDDLVDMYSTMNEPNVVSQMGYIMSRFGFPPSYFNPEMYMKSLLNQAQAHARAYDCIKSFSNKPVGIIYATSVYESMNQDREIEENATYLMNYLFLDVITNGKIFFQERQDLKNKVDFIGVNYYTRTVIDRMKSVDFGLLSLDWTIVDGYGYNCPAGGMSRDSRPVSDFGWETYPEGLLKVLRSIYGRYKLPIIVSENGVADFRDWLRPYYLIGHLYATEKAIQEGVKVLGYLHWSIVDNYEWARGYHMRFGLAETDYETKQFAPRPSMFIFREVVRNGSCEKFYRYLKSPYEIWRI

SEQ ID NO: 41 - protein, 489 aa, synthetic construct
MFSKSFLFGFSLSGFQSEMGLSETDENSDWWKWVHDRNNVRTGIVSGDMPEEGVAYWDLYENYNRAAKNLGANAFRLGMEWTRLFPRATPEIKVDVKEENKDIKDIKVTDETLSSMDKVVNSKAVEHYRKIFKNVKDNNMSLIINVYHWPLPLELHDPILSRESGLTAEKNGWLNHKTVVEFTKYAAYVAWKFDDIADQFSIMNEPNVVFGNGYFNVKSGFPPAYPSPEAGNLSKKHIIEAIGRSYDEMKKFTKKPVGLIYANADFVPKSRDDEDAVELALFNERYSFFDSLRKGDMSWVNVVNDSPVFGEPSSEIRDDLKNKLDWIGVNYYTRNVVKKTETGYTVLKGYGHNAIAGMLSSDNREVSDFGWEVYPEGIYHVLKQYHERYHMPMIVTENGIADSYDKIRPRYLVSHLNEVERAANEGIDVRGYLHWSLLDNYEWSSGFGLKFGILGVNLKTKKIEMRPSALVFKRIAENNGVPEDLKWMA

SEQ ID NO: 42 - protein, 495 aa, synthetic construct
MTRFPKGFMWGISMAAFQYEMGHSKEAVDPNSDWYLWLHNEENKKIGIVSGDVPEMGPGYWDLYKVDHDWAEWLGLNAWRMNPEWSRIFPKSTEEVKVNVESKEGDIIDVEVTEKDLKKLDAMANQKAVKKYKEIFKDINERGIKLIINLYHWPLPIWIHDPIKVRESKLKDGPKGWLEERTVVEFAKFAAYIAWKYEDYAYMWSTMNEPNVTWSIGYLGGNFPPGVNSIEAATKAAYNITQAHARAYDQIKRVIGKKAKVGIIYATTPSEPLTNSEKDIEAAKISDEVSTKWFFNAIVNGVISKNFVGEKIKREDMKEKVDWIGVNYYSRSVVRSKQEPPGYESVAGYGFSCSNMERSNAGLPVSEFGWEIYPEGIRKALKLYKEYEKPLMITENGVADKKDRHRAWYIVSHLYQVSKAIEEDGLKVIGYLHWSLIDNLEWASGFSKRFGLIYVDMSTKKRVPRPSAYLYRDIVTSNEIPEYLLEYSKFPNAIT

SEQ ID NO: 43 - protein, 497 aa, synthetic construct
MIMFPRNFRLGFSLSGFQSEMGVSDVDDNSDWWKWVHDQENIRCGIVSGDLPENGVGHGDLYRNYHSIASYMGVNTARLGIEWTRLFPKASIDTHADTQMDGSDIVHVDVTENVLEKMDSIVNRKALDRYREIFGDLRERGISLIINAYHWPLPLVLHDPIESRSAGLANKRNGWINHTTVVEFVKYAAYVAWKFDDMADQFSIMNEPNVVYGNGLINVKSGFPPSFPSIQAASLSKKHLIEACARSYEEMKRYTKKPVGLIYANSDIQPFSKEDEAFAEQARYQDRYSFFDSLTRGDFSWWVQPNNGEKFSEFSEPHRKDMLHKLDWIGVNYYTRAVVKGNGTGYVTLKGYGHSATPSLPSRDGREVSDFGWEVYPSGLYNLLKQYGERYDLPMIVTENGIADRLDKIRPRYLVSHIHQVERAVHDGINVQGYLHWSLMDNYEWSSGFNMKFGLVGVDLKTKRPQIRPSGLIYKRICENNGVPDDMEWMVPDYPAV

SEQ ID NO: 44 - protein, 490 aa, synthetic construct
MFKDGFLLGFSLAGFQSEMGISDNDENSDWWKWVHDENNIKTGLVSGDQPETGVAYWDLYKTYNQMARSLGSNAFRLGVEWTRLFPKATPEIKVDVEQNNDDIISINVTEKILNDMDNVVNKKALQHYKEIFENVKENGMKLIINAYHWPLPLSLHDPIISRKNGLKNEQNGWLNHRTVIEFVKYSAYLAWKFDYIADQFSIMNEPNVVFGNGYFNVKTGFPPAYPSIEAGNLAKKHIIEAIGRAYDEMKKFTKQPIGLIYANADLVPEKENDKEATKMALFNERHSFFNSLIFGDMKWAKIINEAKEFKEKSDERREDLKGKLDWIGVNYYTRNVIASKESGYVTVKGYGHNAVAGMKSKAGREVSDFGWELYPEGLYHVLKDYHEHYGLPMIVTENGIADSTDSLRPKYLVCHLYEVEKAIKDGMDIRGYLHWSLLDNYEWSSGFGMKFGLLGVDQETKKIQMRPSALVFKYISEHRGIPEDLEWMVK

SEQ ID NO: 45 - protein, 485 aa, synthetic construct
MFPKNFFFGVSLSGFQFEMGGKDAVDDKSDWFLWVRDPVNISSGLVSGDLPEDGPDYWNRYKEDNEIIKDLNMNAVRIGIEWSRIFPTSTTDIDVDVKRNGDSITEVTIDKVALEKLRKRANKDAIKRYRQIMEDIKSKGLHLIVNLNHFTLPIWLHNPLEVRRYGVENSKKSGWFNSEAVVEFAKYAAVVAAEFGDLVDEWSTMNEPQVVSSLGYIQVKAGFPPAYPSFEAFVKTMVNQAQAHARAYDVIKSITSKPVGLIYSFSPVYPADSESAKAVEKAMYFQNYWFMDMITFGKIGKIFENSEEVREDMGRKLDFIGVNYYTRMVVKSVDSFPGWQVLGGYGYGCAPSSKSLDGYLTSEFGWEVYPRGLYDISKAIYDRYGLSMIVTENGIADSTDSIRSYFIVAHLKYLGKALKDGIDVKGYLHWSLIDNYEWAQGFSKKFGLVYVDLKTKKRCLRPSAIVFSKIIEERSVNKFESSVPY

SEQ ID NO: 46 - protein, 504 aa, synthetic construct
MISVHEFPRGFLWGISMAAFQYEMGVSKEAIDPNSDWYVWLHDKTNIEKGIVSGDVPENGPGYWDLFRKDHQLAQWLGLNAWRMNPEWSRIFPKSTKEVKVTVHKDDEGIKDIDISEDALRKLDELANKSAVEHYREVFMDIKGRGMKLIINLYHWPLPLWLHDPIRVRDTNLREGPRGWYDEDAIIEFAKFAAYIAWKFGDLADMWSTFNEPSVTWTLGYIGGNFPPGITNYEAIPRIMINIAQAHARAYDQIKRITGKDVKVGIIYATSPAEPVDEKEENVKAAERANYLANLWLFRAIVEGELDVTPYAIEPKVVKRDDLRDRVDWIGVNYYTRQVVQHQEAPPGFKVVENYGFACKPNSTSAAGRPTSDFGWELYPEGIVSALKLFAKYGRPMAITENGIADAVDRHRPWLLVATLRYLHKAVKEGLNVFGYFHWSLIDNLEWAAGFRMRFGLFYVNMRTKERVPRPSAFIYKHIIEENGIPEYLEEYARIPNFMLGKVE

SEQ ID NO: 47 - protein, 501 aa, synthetic construct
MLPSGFLWGASASGFQFEMGDPAGKYIDPNTDWYVWVHDVDNIAKGIVSGDLPERGIGYWEFYSEDHELAKELGFNTYRIGIEWSRIFPKSTTKIEVEVKRDSYGLIEDISIDESILSELDRIANKEAIEYYRAIIKDIKEKGMRVFVCLNHFTLPLWIHDPIRARRTKLKEGPRGWVDENTVVEFVKYVAYVAWKLGDLVDDWAIFNEPMVVAELGYTMPEAGFPPGVADYGAYLKTLRNLALAHARAYDAIKRWDRVRADPDSKASAYVGLIHNVIPVHPLFKERREDVQAAELLNYTHNKWIVDAIVKGGLDLNLDGKVEVTAPHMANKLDWIGINYYTRMVVRARAKAPFFELVEGLGFACAPHSLSIEGRPTSDFGWEIYPEGLIEAIELMAVYKLPMIITENGIADAEDKLRPGFIVAHIKKLEEAIERGYKIDGYLHWALTDNYEWAKGFSMKFGLVEVDLKTKKRIRRKSAFIYKEIIENNGITSRLEREYLR

SEQ ID NO: 48 - protein, 466 aa, synthetic construct
MFPKDFMFGASLSGFQFEMGNPNDPKEVDPNTDWFVWVREPENLVNGIVSGDLPEYGAGYWKNYEKVHQLAVDFGMDTLRIGIEWSRVFPTSTREVPTGDGMLEALDKIANKEAVEHYRKIMEDMKSKGLKVFVNLNHFTLPLWIHDPISVHKGIPTDKLGWVSDDTPIEFAKYAEYIAWKFSDIVDYWSSMNEPHVVAQLGYFQILAGFPPSYFRPEWYIKSLVNEAKAHNLAYDAIKKYTSRPVGIIYSFIWYDTVNPQDRDIFENAMWLTNWYYIDMVKDKADYIGINYYTRSLIDRLPASGMKFGDFELNWYPLRGYGYACPEGGMSLSGRPASEFGWEVYPEGLYNLIKAIYERYKKIIIVTENGIADEKDKYRSHYLISHLYAVEKAMNEGANVIGYLHWSIVDNYEWAKGYSKRFGLAYTDLEKKIYVPRPSMYIFREIAKTKSIEQFKDYDPYKLMKF

SEQ ID NO: 49 - protein, 466 aa, synthetic construct
MFPKDFMFGVSMSGFQFEMGWGDERDLDPNTDWFVWVREPGNLVNGVVSGDLPEFGAGYWLNYEKIHQLAVDFGMDTIRIGIEWSRIFPTSTESVDVRDPNFLDKLDELANKKAVEHYRKIMEDIKSKGLKLFVNLNHFTLPLWLHDPVAVHYGRPTDKLGWVSERTVHEFAKYVAYMAWKYGDIVDLWSTMNEPHVVSQLGYFSVSAGFPPAYFNPEWYILATKHLAMAHNLGYDMIKRFSDKPTGVIYSFTWYDTLNPNDREILEEAMYLTNWFFMDMVKEKLDYVGVNYYTRTVIDRVEQPLAMGNFNVRWRILKGYGYACDEGGVALSGRPASDFGWEMYPEGLYYVLKAVSERYSKPIIVTENGVADWNDRLRSTHLISHLYYVERALEDGIDVKGYLHWSIVDNYEWAKGYSKRFGLAWTNFQTKTYHPRPSMYIFRDIIRARTTKEFIGFDPYKVRTEL

SEQ ID NO: 50 - protein, 520 aa, synthetic construct
MFPKGFMWGVSLAGFQFEMGNPAGEALDPNTDWFAWVHDRSNIERGIVSGDLPENGIDYWHRFREDHDIAKSLAMNAYRLNVEWSRVFPKPTFSVEVGVTRDDRLIREVEIDEDDLARLDELANKSTVEHYREVVTDLREKGFYVIMNLVHFTLPLWIHDPLTARATKLKKGPTGYADPRFPVEFAKFAAYAAWKFGDLIDMWSTFNEPSVVAEAGFLFKNGKFPPGLFSLDGYKRALINIAQAHVAAYSLIRKFDRKRAYPSSPAEAFVGIIHNMIPFHPLRTNNRRDALAASFSDHLHNRWVLNAIIRGWVDSDIDMKQEPSEVFESYKNKADWIGVNYYTRQVVRGRLSPIGLLAGLPAYPEMVKGYGNECEPRSKSLAGRPTSDFGWEVYPEGLPEVLDIAFEYGRPVLITENGIADREDRLRPHYVALHLKVLEAYIEQRKPDLRGYLHWALTDNYEWADGFRMRFGLYAVDLETKRRLKRPSADVLATIIREGTVPDEYVRKALSAVEGVKVAL

SEQ ID NO: 51 - protein, 517 aa, synthetic construct
MFPKSFLWGVSLAGFQFEMGDPAGEALDPNTDWYVWVHDEYNIREGIVSGDLPEKGIDYWHLFREDHSLAKSLGLNAYRLNVEWSRVFPEPTFSVEVGVEEEDGVKTGIDIDDSDLEKLDSIANKKAVQHYREVVEDLREKGFYVILNLVHFTLPTWIHDPLTARATNAKKGPLGYADPRFPVEFAKFAAYVAASFGDLVDAWSTFNEPSVVTESGFLKRRGKFPPGIFNFDAYKRAMINIAQAHLLAYIAIKKFDRVKAYSDSAESASVGIIHNMIPFHPLDPSRKRDRDASMVTHHLHNSWIPNSLVNGWIDRDFDLKQEPSEVFEKYKSRLDWMGINYYSRSVVKGKVNLLRPVIPFPAFPVLVKGYGFECAPNSQSLAGRPTTDFGWEVYPEGIVEVVKMAMQYNVPLLVTENGVADARDELRPHFLALHLKLLEDALESREISLKGYLHWALTDNYEWADGFRMRFGLFEVDLSSKRRVKRPSADLFARIVSEGTVPDEAVRKAREKLSVNL

SEQ ID NO: 52 - protein, 456 aa, synthetic construct
MFGRDFVFGVSLSGFQFEMGRACPDSVDRGSDWYLWTHDKLNIENGIVSGDFPEDGPNYWNNYGEYHRLAAYSGMKMVRIGLEWSRIFPSPTFDITDVELEGICDRSTLEHYRKIIKDIKERGMKVVVNLNHFTLPLWLHEPVKVNRLSDFTAGGWVDPRSVEQFRKFAALCGRELGDLVDMWSTQNEPNVVAMLGYRNRASGFPPSIIRPELVEVARNNLIEAHLVAYDELKRTSDLPVGLIYAFSWIDGEESAVNLAMKKTQFEFVDRISNKLDFLGCNYYSRMVVESDPSSDNGYRTLRGYGQNCEPNSLSGYGRPTGDFGWEIYPEGLYNILKMICRRYDVPVYVMENGIADMADHYRPYYLISHLKALEKLLEEGYNIKGYLHWSLTDNYEWSSGLGKQFGLIGVDFSDGKLTPRPSFYLFKEIIGQGTVKNFEKMLATPYSIFDTDLLIE

SEQ ID NO: 53 - protein, 455 aa, synthetic construct
MFGEDFLFGVSLSGFQFEMGRNSPESIDPGSDWYVWAHDKLNIENKIVSGTLPESGPDYWTGFEKFHRLAAHSGMKIIRIGLEWSRILPKPTFKRRADEIDDICNAEAIEHYREMIQDIRNLGMKVMVNLNHFSLPIWLHEPIKVNRYSDFTAGGWVDPRTAEEFRKYACLCGRRLGDIVDMWSTENEPDVVAMLGYRNRSSGFPPSIVRPDLVEVARSNLINAHLNAYEELKSQSASPVGLIYAFSWIDGEKSAADSAMEAQLEFIDRIKERTDFLGMNYYSRMVVQPDKDSDRGYRVLPEFGQGCKPNSLSRSGRPTSDFGWEIYPEGLYNILKMTMRRYDIPVFITENGIADGADCLRPYYLISHLHAVERLIEEGFSVKGYLHWSLADNYEWASGFAMRFGLVSVDFEDGSLTSRPSYYLFKEIIEQGSVNSFKKMLKEPYDVFDESLLIE

SEQ ID NO: 54 - protein, 517 aa, synthetic construct
MFPKNFLWGVSLAGFQFEMGDPEGRAIDPNTDWFVWVHDESNIKRGIVSGDLPEKGIDYWHRFREDHDIAKSLGMNAYRLNLEWSRIFPRPTFNINIHVEKDESLLVNASLDEEDLAKLDELADKSAVQHYREVIMDLREKGFIVILNLVHFTLPIWLHDPIQARATNLKRGPLGYADPRFPIEFVKFAAYAAWRFGDLVDIWSTFNEPSVVAEAGFLFKNGKFPPGLFNFDGYRKTLVNIAQAHVLAYKIIKSIDKTKAYPETSSPAEVGIIHNMIPFHPLDPRRHLDVIAARFSDHLHNRWILNAVTRGWVDKDTNMKEEPSEMYAAYKDKVDWIGVNYYSRQVVHGKRNPLSFILGIPVFPEMVKGYGNECVPDSYSMAGRPTSNFGWEIYPEGLAEVLDIAAGYGKPIIVTENGIADREDRLRPHFIALHLKTLEEYIQEKNTVKGYLHWALVDNYEWADGFRMKFGLYAVDLESKKRIRRPSADVLAEIIREGYVPDKYVKEALKVIKPSRQ

SEQ ID NO: 55 - protein, 511 aa, synthetic construct
MKKRFLWGASESGFQFEMGNPEGKFIDSNSDWWAWVHDEENIKKKLVSGDLPEHGINYWELYKKDHELIKKLGMNAYRIGIEWSRILAMSTRDVEVYVKENEDTGLFDSIVVEEEDMKRLLTHINRSAVNHYREIIKDLREQGIKVIVCLNHFTLPIWIHDPIVARRTNLEEGPLGWVDPETVIEFIKYVSVVANLFGDLVDMWATLNEPFVVAEAGYLVPEAGFPPGVAAAEGYLAAIVNLTNAHVRAYDAIKKWDTIKADDDSDEAAFVGIIHNFTPVHPLKKDDERDIQAAKIVDYMRNRWLLNSIVKGEVDLDLNMEISDDEKLKSFRNKLDWIGVNYYVRFVVKHSEKPLVPGFELASFEFVAGYGGECKPRSKSLDNRPTTDFGWEVYPEGLRESLNIAAKYNKPIIVTENGLADAEDALRSWFIVQHIHQLELAIKEDKIDVNGYLHWALTDNYEWAHGFSKRFGLIEVDLKTKERKPRKSSEVFREIAENNGLTDDLTKKYLQ

SEQ ID NO: 56 - protein, 508 aa, synthetic construct
MFPEGFLWGVSESGFQFEMGDPEGKNIDPNTDWFKWVHDPKNIANRIVSGDLPENGIDYWDLYEKDHDLAKGMGMNAYRLGIEWSRIFPRSTREVEVGVEFASDGRIAKVELDDKSLEKLELLSDINALKHYEGIIEDLREKEFKVIVCLNHFTLPLWIHDPIAVRESNLKRGPRGWYDQDTVVEFVKYTAFIAWKLGDKVDMWATFNEPTVVAEMGYFMVESGFPPGVRQVTKVFKNYRRVLLNMINAHARAYEAIKKFDTKKADSTGTSSTVGIIQNIIPVHPYVSHKDSDIRASAFLAKIHNTLFLDAITSGWVDLNFNGVRDTDEVKSDLSDKLDWLGVNYYTRMVVAGGKSILAKLFTGIPVIPTVIKGYGFACEAGSLSKDNRPTSDFGWEMYPEGLEQALIEVSNYGKPIYITENGIADSKDILRGKYILDHLKVLENLIDEKRVDVKGYFHWALTDNYEWAKGFSMKFGLYSVDLESKKRLPRNSVRLLQKVIENGTVKI

SEQ ID NO: 57 - protein, 508 aa, synthetic construct
MGKKFLWGASESGFQFEMGDKEGKSVDSNTDWFVWVHDETNINKRIVSGDLPENGIDYWHLYKKDHELAKSIGMNAYRIGIEWSRILKDTTNIEVYTRENEDTELFDEIVVEEEDMEKLLKLVNNDAVSHYREIIKDLRDKEFKVIVCLNHFTLPLWIHDPISARDSNLEKGPLGWVDKKTVVEFVKYASVVGHLFGDLVDMWATLNEPFVVATIGYLMEKEGFPPGVSSPEGFFNVAYNITNAHVRAYDALKKWDTVKADDDSEAPAYVGLIHNMSPAHPLDPSNEDDVRAAEILDYILNRWIINATLKGEVDLDLDFEISEEEKLSSYRHRLDWFGLNYYTRNVVKKAENPLVPGLEIASFELVEGYGSFCKPNSKSRDNRATTDFGWEIYPEGLRELLNELSEYNIPIYVTENGIADATDRLRPKYLVEHINQLELAIKKDKVKVNGYFHWALTDNFEWAAGYKMRFGLIEVNLNTKERILRKSAQIFKEIAENNGLTEKLKQTI

SEQ ID NO: 58 - protein, 505 aa, synthetic construct
MFPEDFLWGVSISGFQFEMGNKERKYIDDKSDWFVWVHDQENIRTKLVSGDFPEDGINYWELYEKDHQLARNLGLNAFRLNIEWSRIFPEPTFKIDAGVEREDNGYISKIDIAQKDLEELENNANLEALKHYEQIIKDITDKEMKVLVCLNHFTLPLWIHDPIVARKTKIKKGALGWIDEKTVIEFCKYAAFIAWKLGNLVDFWITFNEPMVVVEMGYAQKASGFPPGVENLNAMNKAAINLAIAHARSYDLIKKFDTYKADNKSPSSATVGIVSNMIPFEPMNKKSKADVSAANFMDFLHNRFIIEAITSGQLIERLQERKNNKKERRHLANRLDWIGINYYSRAVVRGKFSLLAKSFIGIDYLPELVEGYGLYCKPNSLSIDKRETSDFGWEFYPEGLQEVIEKTSKFKLPVYITENGVADSEDKLRSKYIFEHLRIVEDILNTRKFNLKGYFHWSLIDNYEWAKGFSMKFGLFEVDLKTKERKMRKSAMTLRNIVEQKTTYF

SEQ ID NO: 59 - protein, 956 aa, synthetic construct
MADRAWTASWIWGGETESPRNEWRCFRRSFECPADFGGEARLHISADSRYVLYVNGERIGRGPVRSWPKEQFYDTYEVGSLLRAGAPNTVAVLALHFGVSNFYYLRGKGGLIAELEAGGRPLLATDASWRTRKLAGQASESPRMSCQQGFAEAIDAREADGDWTEPAYDDAAWAPARPVGPAGTAPWLRLVPRDIPFLTEEKLYPSRVLSVRRVKAPAYTAALDLRNQMVPESANHANPIGYCGYLATVLRMERSGRATIGFPGGAKPGDLYVDGRRIESWSGRVPERYYELNLEAGDHLVVYDVTSTDHGGSFHIGIDSEVPFQLTNPLGEKETSVPLATAGPFDHTEHIDYLPGREVRRDHPDYASLKHASTVQELLAYRSWLRPVEAGLYTEADAFGSSVWRTVAEKRAVPASLANAILPVPEPAVLPYFKDGDCELTIDFGKERSGFVGFEIEAPAGTVVDLYGVEYLRDDYIQHTYGLDNTVRYTCREGRQCYLSPVRRGFRYLIVTVRGARDPVKLHEIYINQSSYPVAENGDFRCSDSLLNEIWEISRHTTRLCMEDTFVDCPAYEQVFWVGDSRNEALVNYYVFGATDIVKRCLNLVPGSADQTPLYLDQVPSAWNSVIPNWTFFWAIACREFAAHTGDLTFARSIWPAVQRTLKHYLTHLNADGLLAIRGWNLLDWAPIDQPGDGIVTHQNLFLVKALRDAAQLGELAGAQEEAEAFRDSADKLLRAIQAHLWDEPRQAYLDCIHADGRRSEIFSMQTQVVAYLCGAAEGDRRETLERYLVSPPPEFVQIGSPFMSFFYYEALVKSGRYERMLDDIRKQYGSMLAHGATTCWEMYPNFAENRADPDMLTRSHCHAWSAAPGYFLGESILGVKRLDDGWQSVEIAPQPCGLTWARGSVPLPQGGQVAVSWRVDGGRMKLRAEAPEDVRLSVRWPEGMEGEAEQATYIR

SEQ ID NO: 60 - protein, 960 aa, synthetic construct
MTSRDWQASWIWGGAEDSPRNEWRCFRRAFDVPADVDRSARLHISADSRYVLYVNGTQVGRGPVRSWPKEQFYDTYEIGHLLRPGGANTIAALVLHFGVSNFYYIRGRGGLLAEAEANGRVLAATDGEWTTERLGGQQAGSPRMACQQGFAEVIDARAGDDRWIEPGFDDGHWAKARTIGAAGTAPWLTLVPRDIPFLTEEKFYPASVMSLHRVQAPAFVAALDLRNQMVPESANHANPIAYCGYLATELVLERDGDVTIGFPGGRMNRLLVDGVPYETWRGEQPERYYDLKLSAGGHLVLVDVTVGDHGGSFHIGVDGDVPFALRAPIGEAGETAAPFVTVGPFDHSEWIDHRPGHREMRKDHPDYESLRTCASSAELAAYRSWIRPVDPSMYTEDDVFGASVWRTVAERRSVPLSLQNAVLPVPEPAALPGFADGDVELVIDLGRERSGFVGFEIEAPAGTIVDGYGVEYVRDEYIQHTYGLDNTFRYVCRAGRQRYLSPVRRGFRYLILTIRGETERVKLHEVYLNQSTYPVAEIGRFRCSDAQLNEIWEMSKHTTRLCMEDTFVDCPAYEQVFWVGDSRNEALVNYYVFGATDIVKRCLNLVPGSADMTPLYVDQVPSAWSSVIPNWTFFWIIACEEYAAHTGDRAFARSIWPAVRHTLDHYLQHLDDSGLLFIRGWNLLDWAPIDQPGDGIVTHQNLFLVKALRQAARLAQAAGADAEAPRYEERADALARAINAKLWDEARSAYLDCIHADGRRSSVFSMQTQVVAYLCDVAEGERRSAMEDYLLSPPPAFVQIGSPFMSFFYYEALEKSGNHRHLLDDIRKHYGLMLDHGATTCWEMYPNFDENRANPNQLTRSHCHAWSAAPGYFLGASILGVKRSDAGWQSVEIAPQPSGLTWAEGAVPLPEGGHVAVRWRLEEGRLNLRVEAPSDVRVDVRLPEGMEGDVERVTFERLWD

SEQ ID NO: 61 - protein, 955 aa, synthetic construct
MKDRTWKAAWIWGGLEASPRNQWRCFRHTFHCPADYEGAAQLRISADSRYCLYVNGQLVGRGPNRSWPSEQSYDPHEVGHLLRPGKMNSIAVLVLHFGVANFYYIRGRGGLIAELLSGNQILAATDGQWKTATLAGGQPNSPRMCCQQGFAETIDARMQQGDWTSTTFDDAAWEFASPIAPSEAPWGSLIERDIPFLTEEKYYPSTILSLHRVRPATYTAALDLRNQMVPESANHANRIEYCGYLATKLKLTEKGTVTVGFPNGMRTDYLLVDGTLQEEWYGEQPERYYRLHLEAGEHLLLIDITCCDHGGSFHIAADGDIPFSFLSPQGDRTVPFITIGPFDQTERVDHQEDHLLRRDHPLYLSLKTAKTTEDLLQAEPWIRPMEEKLYTEEDVFGANVWRTIAERRALPLSMQNAILPVPEPAVLPLFEDGDCELVIDLGKERSGYLGFELEAPEGTIVDVYGVEYRHEHYTQHTYGLDNTFRYHCKAGRQHYLSPVRRGLRYLILTVRGNKTPVKLYEVFMNQSTYPVAETGQFTCSDPLLNQIWEISRHTTRLCMEDTFVDCPAYEQVFWVGDSRNEALVNYYVFGTTDIVKRCLSLVPKSADMSPLYVDQVPSAWSSVIPNWTFFWITACEEYIAHTLDIGFAKEIWPAVRFTLVHYIQHLDPSMLLNIKGWNLLDWAPIDQPNEGIVTHQNMFLVKALRTAIALAETAGLASEAADSGFAAIAGEMAAAINRQLWSEERKAYLDCIHTDGRASKIFSMQTQVVACLCGLVEGDRKSVIEGYLCSPPAEFVQIGSPFMSFFYYEALEKAGKYNLMLEDIRHHYGKMVEHDATTCWEMYPDFAENRANPKQLTRSHCHAWSASPGYFLGASILGIKRLDPGWSSVEIAPQPCDLQWAKGAVPLPQGGQIEVSWSMHKSVMTLRVEAPEDVDISIRWPEGIEGQLEQITYLR

SEQ ID NO: 62 - protein, 978 aa, synthetic construct
MTTHDWTASWIWGGGESSPRNEWRCFRKQFECPADFAGQAQVRLCADSRYVLYVNGMRVGRGPVRSWPHEQFYDTYEIGHLLRPGAVNTLAILVLHFGVANFYYIRGRGGLLAEVAAGDVALAATDGEWRTRPLAGFSRAAPRMSCQQGFSEIIDAREEDGDWSGEGYDDSAWERAVVIGPVGTAPWTKLVPRDIPYLTEEKLYPAAILEVNRVETPAFMAAIDMRNQFVPESADHANPIRYCGYLATVLRLERAGTVTVGLPTGLPSGSFYVGGVAVKEWRGEQPEQYAELALPAGDHLLLLDATASNHGGSFHFGLDADAPFSLRSPLGELEAGAGTPFATYGPFDHVEYIDHQGARELSCDHADYLALAAVSSAAELEAYREWARPLDPGMYTEADVFGLSVWRTRVERKPVAPELVRAVLPVPEPAALPRFDVGDCELVIDLGKEQSGFIGFEIEAPAGTILDLYGVEYRRDDYTQHTYGLDNTLRYVCREGRQRYLSPVRRGLRYLIATVRSAAGGDPSADVSNNVSGNASGKREPVKLHEIYIHQSNYPVANQGTFRCSDPLLNDIWEISRHTTRLCMEDTFVDCPAYEQVFWVGDARNEALVNYYVFGATDIVKRCLNLVPGSSEMTPLYVDQVPSAWSSVIPNWTFFWVIACEEFIGHTGDRAFAQRIWPAVRHTLVHYLTHLDKDGLLNMKGWNLLDWAPIDQPNDGIVTHQNLFLVKALRKAAAIAEAAGLAHEAEEAGFAASSDSLAEAINGRLWDDTRRAYLDCIHPDGRRSDIFSMQTQVVAYLCEVAEGERRATIDGYLAAPPAEFVQIGSPFMSFFYYEALVRSGRHAQILADIRRNYGAMIEHEATTCWEMYPNFAENRANPKQLTRSHCHAWSAAPGYFLGASALGVNRKDAGWQTVEIAPQPSDLAWASGSVPLPQGGHIGVRWTRSDARMKLRVEHPREVSVNVVWPEGLEGELEEVVY

SEQ ID NO: 63 - protein, 420 aa, synthetic construct
MHVIITAVGSAGDVHPFVGIGRTLAARGHRITFCASAAFAPLIERCGFKFLPLGTREEYDEVMADPALWHPRTSLPTLWKTVGSTLREQYRLLEQACDDETVMLGSLWAFSARLLQEKHGIPLVTAQVSPFGFWSAAAPSTHPPGANLPAFVPYPVKRLLLGLIERAFLDRVMAPELNSFRRELGLPPVKRIISQWISSPDRVLGLFPEWFAPVQHDWPAQTVLTGFPLFDESGFQPPDAELEDFLNEASPVVFTPGSTMVNSEQYFAAAVQALKLINRRGVFLTSQPVDPAVSSEGRILVRRYVPMSRILPHSAALVHHGGVGTTALAMAAGVPQIATPFAHDQFDNAARMERLGCGLRVFPPASAESLATALDKVLNSQEVRASCDRYRTLIPSGDSASEAAALQIEALARTARYRAA

SEQ ID NO: 64 - protein, 420 aa, synthetic construct
MHVIITAIGSAGDVHPFVGIGRTLAARGHRITFCASAAFAPLIERCGFKFLPLGTREEYDEVMADPALWHPRTSLPTLWKTVASTLREQYRLLEQACDDQTVMLGSLWAFSARLLQEKRGIPLITAQVSPFGFWSAAAPSTHPPGANLPAFVPYPVKRLLMGVIERTFLDRVMAPELNRFRRELGLPPAKRIISQWISSPDRVLGLFPEWFAPVQRDWPAQTVLTGFPLFDESSFQEPDAELDDFLNEASPVVFTPGSTMVNSEQYFAAAVQALKLINRRGVFLTSQPVDPTVATEGRILVRRYVPMSRILPQSAALVHHGGVGTTALAMAAGVPQVATPFAHDQFDNAARMERLGCGLRVFPPASAESLATALDKVLNSQEVRASCDRYRTLIPSGESACEAAARQIEAIVAEPGYRAR

SEQ ID NO: 65 - protein, 420 aa, synthetic construct
MHVIITAIGSAGDVHPFVGIGRTLAARGHRITFCASAAFAPLIERCGFNFLPLGTREEYDEVMADPALWHPRTSLPTLWKTIASTLREQYRLLEQACDDETVMLGSLWAFSARLLQEKHGIPLVTAQVSPFGFWSAAAPSTHPPGTNLPAFVPYPVKRLLLGVIERAFLDRVMAPELNSFRRELGLPPTKRIISQWISSPDRVLGLFPDWFAPVQHDWPAQTVLTGFPLFDESGFQPVDAELDDFLNEASPVVFTPGSTMVNSEQYFTAAVQALKLINRRGVFLTSQPVDPTVSSEGRILVRRYVPMSRILPHSSALVHHGGVGTTALAMAAGVPQIATPFAHDQFDNAARMERLGCGVRVFPPASAESLAAALDKVLNSQEVRASCDRYRTLIPSGESASEAAALQIEALARTARSRAA

SEQ ID NO: 66 - protein, 420 aa, synthetic construct
MHVIITAIGSAGDVHPFVGIGRTLAARGHRITFCASAAFAPLIERCGFNFLPLGTREEYDAVMADPALWHPRTSLPTLWKTIASTLREQYRLLEQACDDQTVMLGSLWAFSARLLQEKHGIPLITAQVSPFGFWSPAAPSTHPPGTNLPAFVPYPLKRLLLGMIERTFLDRVMAPELNSFRRELGLPPVKRIISQWISSPDRVLGLFPEWFAPVQHDWPAQTVLTGFPLFDESGFQQPDAELEDFLNEASPVVFTPGSTMVNSEQYFAAAVQALKLINRRGVFLTSQPVDPTVSSEGRILVRRYVPMSRILPHSSALVHHGGVGTTALAIAAGVPQIATPFAHDQFDNAARMERLGCGLRVFPPASAESLATALDKVLNSQEVRASCERYRTLIPSGESACEAAALQIEALARTAKSRAA

SEQ ID NO: 67 - protein, 420 aa, synthetic construct
MHVIITAIGSAGDVHPFVGIGRTLAARGHRITFCASAAFAPLIERCGFKFLPLGTREEYDAVMADPALWHPRTSLPTLWKTVASTLREQYRLLEQACDDETVMLGSLWAFSARLLQEKHGIPLVTAQVSPFGFWSAAAPSTHPPGTNLPAFVPYPVKRLLLGFIERAFLDRVMAPELNSFRRELGLPPTKRIISQWISSPDRVLGLFPEWFAPVQHDWPAQTVLTGFPLFDESGFQQTDAELDDFLNEASPVVFTPGSTMVNSEQYFAAAVQALKLINRRGVFLTSQPVDPTVSSEGRILVRRYVPMSRILPHSSALVHHGGVGTTALAMAAGVPQIATPFAHDQFDNAARMERLGCGVRVFPPASAESLAAALDKVLNSQEVRASCDRYRTLMPSGDSACEAAALQIEALARTATSRVA

SEQ ID NO: 68 - protein, 420 aa, synthetic construct
MHVIITAIGSAGDVHPFVGIGRTLAARGHRITFCASAAFAPLIERCGFKFLPLGTREEYQAVMADPALWHPRTSLPTLWKTIAGTLREQYRLLEQACDDDTVMLGSLWAFSARLLQEKHGIPLVTGQVSPFGIWSPAAPPIHPPGANLPAFVPYPLRRLLLGVIEHTVLDRLMKPELNSFRRELGLPPVKRIISQWISSPDRVLGLFPDWFAPVQQDWPAQTVLTGFPLFDESGFQEPDAELEDFLNEASPVVFTPGSTMVDAEQYFAAAAQALKLINRRGVFLTSQPVDPALSSDGRILVRRYVPMSRILPHCSALVHHGGVGTTALAFAAGVPQIATPFAHDQFDNAARMERLGCGLRVFPPASAESLATALDKVLNSQEVRASCERYRKLMPSGDSACEAAALQIEALARTARSRVA

SEQ ID NO: 69 - protein, 422 aa, synthetic construct
MHVIITAIGSAGDVHPFVGIGRTLAARGHRITFCASAAFAPLIERCGFKFLPLGTAEEYEAVMADPALWHPRTSLPTLWKTVASTLREQYRLLEAACDDETVMLGSLWAFSARLLQEKHGIPLITAQVSPFGFWSAASPSVHPPGTNLPAFVPYPVKRLLLGLIERAFLDRVMAPELNRFRRELGLPPTKRIISQWISSPDGVLGLFPEWFAPVQSDWPAQTVLTGFPLFDESGFQQTDAELDDFLSEASPVVFTPGSTMVNGEQYFAAAVQALKAINRRGVFLTRQPVDPTVAAAGQILVRRYVPMSRILPHSAALVHHGGVGTTALAMAAGVPQVATPFAHDQFDNAARMERLGCGVRLSPPASAESLAAALDKVLNSPEVRANCARYRALIPSGDSACEAAALQVEALALSRTKTSRVA

SEQ ID NO: 70 - protein, 420 aa, synthetic construct
MHVIITAIGSAGDVHPFVGIGRTLAARGHRVTFCASAAFAPLIERCGFNFLPLGTREEYDAVMADPALWHPRTSLPTLWKTIASTLREQYRLLEQACDDETVMLGSLWAFSARLLQEKHGIPLVTAQVSPFGFWSPAAPPTHPPGANLPAFVPYPLRRLLLGMIERAFLDRVMAPELNSFRRELGLAPVKRIISQWISSPDRVLGLFPDWFAPVQHDWPAQTVLTGFPLFDESGFQPPDAELEDFLNEASPVVFTPGSTMVNGEQYFAAAAQALKLINRRGVFLTSQPVDPALSSEGRILVRRYVPMSRILPHCAALVHHGGVGTTALAMAAGVPQIATPFAHDQFDNAARMERLGCGLRVFPPASAESLATALDKVLNSQEVRASCERYRKLIPSGDSASEAAALQIEALARTARSRAA

SEQ ID NO: 71 - DNA, 1259 bp, synthetic construct
  source 1..1259: mol_type = other DNA
atgcacgttattataacagctgtaggatcagctggcgacgtgcacccgtttgttgggattggccgtaccctggctgcgcgtggtcatcgtattaccttttgcgcgtctgcggcatttgcaccgctgatcgagcgttgtggtttcaaattcctgccgctgggcacccgtgaggaatatgatgaagttatggcggatccggccctgtggcacccgcgtaccagcctgccgaccttatggaaaaccgtcggtagcaccctgcgtgagcagtaccgcctgttggaacaggcgtgtgatgatgaaaccgtaatgttgggttccttgtgggcatttagcgcgagattgttgcaagagaagcacggcatcccgctggttaccgcgcaggttagcccgttcggtttttggagcgctgcggcgccgtctacgcatccgccgggcgcaaatctgccggccttcgtgccgtatccggtgaagcgcctgttgctcggtctgattgagagggcatttttggaccgtgtgatggcaccggaactgaacagctttcgtcgcgagttgggcctgccgccagtgaagcgtatcattagccaatggattagtagcccggaccgcgtcttgggcctattcccggagtggtttgcgccagttcagcacgactggccagcccagaccgtactgactggtttcccgctttttgacgagtccggcttccagccgcctgatgctgagctggaagattttctgaacgaggccagtccggtggtgttcaccccgggttcgacaatggttaattcggagcaatacttcgcggcggcggtccaggctctgaaactgattaaccgtcgtggcgtgttcctgacctcccaaccggtggacccggctgttagcagcgaaggtcgtatcctggttcgtcgatacgtgccgatgtctcgtatcctgcctcacagcgctgcgttggtccaccatggtggtgtgggcacgaccgcgctggccatggcggccggtgttccgcagatcgcaacccctttcgcgcatgaccagtttgacaacgcagcgcgtatggaacgcctggggtgcggtctgcgtgttttcccgccggcgtccgccgaaagcctggcaactgcgctggataaagttctgaactcgcaagaagtgcgtgcgagctgcgatcgttaccgcacgttgatcccgtccggcgacagcgcgtctgaagccgctgcccttcaaatcgaggccctcgctagaacggctcgctatcgcgcggc

SEQ ID NO: 72 - DNA, 1260 bp, synthetic construct
  source 1..1260: mol_type = other DNA
atgcacgtaataattacagctataggatcagcgggagatgtgcatccgttcgtgggcattggtcgtacgttggcggctcgcggccatcgtatcaccttctgcgcgtctgcggcgttcgccccgctgatcgagcgctgcggttttaaattcttgccactcggcacccgtgaggaatacgacgaggtgatggccgatccggcactgtggcacccacgtacctctctgcctaccctgtggaaaaccgtggcttcgaccttaagagagcaataccgcttgctggaacaagcctgcgacgatcaaacggtgatgctgggcagcctgtgggcattcagcgcgcgcttgctgcaggagaagcgcggcattccgcttatcaccgcgcaggtgagcccgttcggcttctggagcgcggcagcaccgtccacccacccgcctggcgcgaatctgccggcgtttgttccgtacccggttaaacgtctgctgatgggtgtgatcgagcgtacctttttggaccgcgtgatggctccggaactcaaccgctttcgtcgtgagctgggccttcctccggcgaaacgtattatcagccagtggattagcagcccggatcgcgtgctaggtttgtttccggaatggtttgccccggtccagcgcgattggccggctcaaaccgtactgacgggtttcccgctgttcgatgagtcctcttttcaagagccggacgccgaactggacgacttcctcaacgaagcctcgccggttgtttttaccccgggcagtacgatggttaatagcgaacagtattttgctgcagcagttcaggcgctgaagctgatcaaccgtcgtggtgttttcctgaccagccagccggttgatccgaccgtggcgaccgagggtcgtatcctggttcgtcgctatgttccgatgagccgtattctgccgcaatccgctgctctggtccaccacggcggtgtgggtacaaccgcgttagctatggcagcgggtgtcccgcaggttgcgactccgttcgcgcatgatcagtttgacaacgcagcccgtatggaacgtttgggctgcggtttgcgtgtattcccgccagcgtcggctgagagcctggcgactgcgttggacaaggtgctgaacagccaagaggttagggcgtcttgtgaccgttaccgtacgctgatcccgtccggtgaatccgcgtgtgaagcggcggcgcgtcagattgaagcaattgttgcggaaccgggttatagagcacgg

SEQ ID NO: 73 - DNA, 1260 bp, synthetic construct
  source 1..1260: mol_type = other DNA
atgcacgtaataattacagctataggatcagcaggcgatgttcacccgttcgtgggtattggtcgtacactggctgcgcgtggccaccgtatcaccttttgcgctagcgcggccttcgcgccgctgattgagcgttgtggtttcaactttctgccgctgggcacgcgtgaggaatatgatgaggtaatggctgatccggccctgtggcacccgcgtacgagcctgccgactctctggaagaccattgcatccaccctgcgcgagcagtaccgcctgctggaacaggcgtgcgacgacgaaaccgtcatgctgggtagcctgtgggcattcagcgcgcgtttactgcaagaaaaacacggcatcccgttggtcaccgcgcaggttagcccgtttggtttctggtcagccgccgcgccaagcacccatccgcctggtacgaacctgccagcgttcgttccttatccggtgaaacgcctgctgttgggtgttattgagcgcgcatttttagaccgtgtgatggcaccggaactgaacagctttcgtagagagctaggcctgccgccgacgaaacgtatcatcagccaatggatctcttccccggaccgcgttttgggcctgttcccggattggtttgctccggttcagcacgactggccggcgcagaccgtcttgaccggttttccattgttcgacgagagcggcttccagccggtcgatgcagagttggacgattttttgaatgaagctagtccggttgtttttaccccgggttccaccatggtgaatagcgaacaatatttcaccgcggctgtgcaagcgcttaagctgatcaaccgccgtggtgttttcctgacctcccagccggtggatccgaccgtgagctcggagggtcgtattctggttcgtcgctacgttccgatgagccgcatcctgccgcacagcagcgccctggtgcatcatggtggtgtaggcaccaccgcgcttgccatggcggcgggtgtgccgcagattgcaactccattcgcgcatgatcagtttgacaatgcagcacgtatggaacgtttgggctgcggcgtgcgtgttttcccgccagcgtctgcggaatcgctggcggcggctctggacaaggtgctgaactcgcaagaggtgcgtgcttcttgtgaccgataccgtaccctcatcccgtccggcgaaagcgcgtctgaggcggctgccttgcaaattgaggcgctggcgcgtaccgcccggagccgcgctgcg

SEQ ID NO: 74 - DNA, 1260 bp, synthetic construct
  source 1..1260: mol_type = other DNA
atgcacgtaataattacagctataggatcagctggtgatgttcacccgttcgtcggaatcggccgtacgctggcggctcgtggtcatcgtatcaccttctgcgcatctgcagcgtttgcgccgcttatcgagcgctgcggtttcaacttcctgccgctgggtacgcgtgaggaatatgatgcagtcatggcggatccggctctgtggcatccgcgtaccagcttaccgaccctgtggaaaaccattgcgagtaccttgagagagcaataccgtttgctcgagcaagcctgcgacgaccaaacggtgatgctgggctccctgtgggcattctccgcaagactgctccaagaaaagcacggcattccgctgattaccgcgcaggtgagcccgtttggtttctggagcccggcggctccgtcgacgcacccgccaggtaccaacctgccagccttcgtgccgtatccgctgaagcgtctgttgctgggcatgattgagcgcaccttcctggatcgtgtaatggcaccggagctgaactcttttcgtcgcgaactgggcctgccgccggtaaagcgcatcatcagccaatggatcagcagcccggaccgcgtccttggtctgttccccgagtggtttgcgccggtgcagcacgattggcctgcgcaaaccgtgttgaccggtttcccgttgtttgacgaaagcggcttccagcagccggacgcagagctggaagattttctgaatgaagctagcccagttgtttttaccccgggttccacgatggttaattcggaacagtacttcgcggcggcggttcaggcgttaaagctgatcaaccgtcgtggcgtgtttctgacttcacaaccggttgatccgaccgttagctccgaaggtcggattctcgtgcgccgctacgtcccgatgagccgtatcctgccacatagctctgcgctggtgcaccacggcggtgtgggcactacagccttggcgattgcggcaggtgttccgcagatcgcgaccccgtttgcgcatgaccagtttgacaatgcagcccgtatggaacgtttgggttgtggcctgcgtgtgttcccgccggcgtctgctgaatccctggccaccgctctggacaaagttttgaacagccaggaggtgcgtgcgagctgtgaacgctatcgcaccttgatcccgagcggtgagtccgcgtgcgaggctgcggctctgcagattgaggcgctagctcgcactgccaaaagccgtgcagcc

SEQ ID NO: 75 - DNA, 1260 bp, synthetic construct
  source 1..1260: mol_type = other DNA
atgcacgtaataattacagctataggatcagcgggtgacgtgcacccgtttgttggtattggtcgtacgttggcggcgagaggtcatcgtatcaccttttgtgcatccgccgcgtttgcgcctttgatcgagcgctgcggctttaaattccttccgttgggcacccgtgaggaatatgatgcggtgatggccgatccggcgctgtggcatccgcgtaccagcctgccgactctgtggaagaccgtggcgtctacgctgcgtgagcaatatcgtctcctggaacaggcctgcgatgacgagacagtcatgctgggcagcctgtgggcattcagcgctcgtcttttacaagaaaaacacggcatcccgctcgtcaccgcacaagtttccccgttcggcttctggtccgccgcggcgccaagcacccacccgcctggcaccaacctgccggcgttcgtgccgtatccggttaagcgcctgctgctgggtttcatcgagagagcattcttggaccgcgtgatggcgccggaattaaacagttttcgtcgcgagctgggcctgccgccaacgaaacgtattatcagccagtggattagcagcccggatcgtgtcctgggcctgtttccggagtggtttgctccggtccaacatgattggcctgcgcagaccgttctgaccggtttcccgctgttcgatgagtctggcttccagcagaccgatgcagaactggacgacttcttgaatgaggcgagcccggtggtgtttaccccgggttccaccatggttaattcggaacagtacttcgctgcggcggttcaagcactgaaactgattaaccgccgtggtgtgttcttgacctctcagccggttgatccgacggtttccagcgagggtcgtatcctggttcgtcgttacgtgccgatgagcagaatcttgccgcactcttcggctctagttcaccacggcggtgttggtacgaccgcgttggccatggcggcaggcgttccgcagattgctaccccgtttgcgcatgaccagtttgacaacgccgcacgcatggaacgcttggggtgcggtgtgcgtgttttcccaccagcgtcggcggaaagcctggcagctgcgctggacaaggtgctcaattcacaagaggtgcgcgcaagctgcgaccgctaccgcaccctgatgccgagcggtgacagcgcgtgtgaagcggctgccctgcaaattgaggctctggctcgtactgcgacctctcgtgtagca

SEQ ID NO: 76 - DNA, 1260 bp, synthetic construct
  source 1..1260: mol_type = other DNA
atgcacgtaataattacagctataggatcagctggcgatgttcacccgttcgttggtattggtcgtacgctggctgctcgcggtcatcgtattaccttttgtgcgtctgcggcatttgcgccactcatcgagcgctgcggcttcaaattcttaccgctgggcacgcgtgaggaatatcaggcagttatggcagatccggcgctgtggcatccgcgtacctccctcccgaccctgtggaagaccatcgcaggcaccctgcgtgaacaataccgcttgttagagcaagcctgcgatgacgacaccgtcatgctgggctctctatgggcattcagcgcgcgcttgctgcaagagaaacacggtatcccgttggtcaccggtcaggttagcccgtttggtatttggtcaccggcggctccgcctattcacccgccgggtgctaatctgccagcgtttgttccgtatccgctgcgtagattgttactgggcgtgattgagcacaccgtgcttgacagactgatgaaaccggaactgaactcctttcgtcgcgagttgggcttaccaccggttaaacgtatcatcagccagtggattagtagcccggaccgcgtgctgggcctgtttccggactggttcgctccggtgcagcaggattggcctgcccaaaccgtgttgaccggcttcccgctgttcgacgaaagcggtttccaggaacccgacgcggaactggaagatttcctgaacgaggcgagccccgtggttttcactccgggttccaccatggttgacgccgagcaatacttcgccgcggcggcgcaggcgttgaagctgatcaaccgtcgtggtgtctttctgacctcccaaccggttgacccggctctgagctctgatggccgtatcctggtacgtcgttatgttccgatgagccgtatcctgccgcattgtagcgcgctcgtgcaccacggcggtgtcggaactacggccttggcctttgcggcgggtgtgccgcagattgcgacaccgttcgcgcatgatcaatttgataacgccgcgcgcatggaacgtttgggctgcggtctgcgcgtgtttccgccagcttcggctgagagcctggcgaccgcactggataaggtgctcaatagccaagaagttcgtgcatcctgcgagcgctaccgtaagctgatgccgagcggtgacagcgcgtgcgaagcagcagcgctgcagatcgaggccctggcgcggacggctcgtagcagagtggca

SEQ ID NO: 77 - DNA, 1266 bp, synthetic construct
  source 1..1266: mol_type = other DNA
atgcacgtaataattacagctataggaagtgcgggcgatgttcacccgtttgttggcattggtcgtacgttggcggcgcgcggtcatcgtatcacgttttgcgcaagtgccgcgtttgctccgctgattgaacgttgtggcttcaagttcttgccgctgggcaccgcggaggaatatgaagcagttatggcagacccggcattgtggcatccgcgcaccagcctaccaacgctgtggaaaaccgtggcgtccaccttgagagagcagtatcgtttgctggaagcggcttgcgacgatgagactgttatgctgggcagcctttgggctttcagcgcgcgtctgctgcaagaaaaacacggcattccgctgatcaccgcgcaggtaagcccatttggtttctggtcagcagcttccccgagcgttcacccgccaggcacgaacctgcctgcgttcgtgccgtatccggtgaaacgtcttctgctgggtctcatcgagcgtgccttcttggatcgtgtgatggcacccgaactgaaccgttttagacgtgagctgggcctgccgccgaccaaacgtatcatctctcagtggatttcgtcccccgacggcgtcctcggcctgttcccggagtggtttgccccagtccaaagcgattggcctgcccaaactgttctgactggtttcccgttatttgacgagagcggtttccagcaaaccgatgccgagctggacgactttttgtctgaggcgagcccggttgtttttaccccgggtagcaccatggttaatggtgaacaatacttcgcagcggcggtgcaggcgctgaaggcaattaatcgacgtggcgtcttcttgacccgtcagccggtggatccgaccgtggctgctgcaggtcagattctggttcgccgctacgtcccgatgtcccgcatcctgccgcacagcgctgcgttggttcaccatggtggtgtgggtacaaccgctctggcaatggccgctggcgtgccgcaggtcgcaaccccgttcgcgcatgatcagtttgacaacgcggcgcgcatggaacgcctggggtgcggtgtgcggctgtcaccgccggcttccgcggagtcgctggcggcggcgttggacaaggtgttgaacagcccggaggttcgtgcgaattgtgcccgttaccgcgcgctaatcccgtctggtgacagcgcatgcgaagcagcggcgttacaagttgaagccctggctttgtctcgcaccaagaccagccgtgtggcg

SEQ ID NO: 78 - DNA, 1260 bp, synthetic construct
  source 1..1260: mol_type = other DNA
atgcacgtaataattacagctataggatcagctggcgacgttcacccgtttgttggtattggtcgtaccctggccgcgcgcggtcaccgcgtgactttctgcgcttccgcggccttcgccccactgatcgagcgctgcggtttcaactttttgccgttgggcaccgcggaggaatacgacgccgtgatggcggacccggcactgtggaatccgcgtacttcgctgccgacgctgtggaagaccattgcatccaccctgcgtccgcaatatcgtttgctagaacaagcgtgcgatgacgataccgtaatgctgggtagtctgtgggcattcagcgcgcggttgcttcaagaaaagcacggtatcccgctggttaccgctcaggttagcccgttcggctttttaagcgcagcagcgccaccgacgcatccgaaaggtgcaaatctgccagcgttcttgccgtatccggtgcgtcgcctgcttttgggcatgattgagcgtgcggttttagatcgtgtttgcggccctgagctgaactcttttcgtcgcgaactgggactagccccgaccaaacgtatcatcggccagtggattcatagcccggatggcgttctgggcctgttcccggactggtttgcaccgccgcagcacgactggccggcccaaaccgttctgaccggtttcccgctgtttgacgagtcgggtctgcaaccgcttgacgcagagctggaggatttcctgaacgagggcagcccggtcgtgttcacgccgggtagcaccatggttgatggtgaacagtatttcaccgcggctgcgcaggcgctgaagctgattggccgcagaggcgtgtttttgaccagccagagcgtcgacccgtccgtgagcagcgaaggtcgcattctggtgagacgctacgtgcccatgagccgtatcctgcctcattgtgctgcgctcgttcaccacggcggtgtgggcaccacggcgctggctatggcggcgggtgtcccgcaaatcgcgaccccgttcgcacatgatcagtttgataatgcagcgcgtatggaacgtttgggttgtggcctgcgtgtgtttccgcctgcgtcagcggaaagcctggctaccgcactggacaaaatcctcaactcgccggaagttcgtgcgtcttgtgagcgctaccgtaccttgatcccgagcggtgattccgcctgcgaagcggctgcgctgcagattgaggcactggcgcgcacggcgcgttctcgtgctgcc

SEQ ID NO: 79 - protein, 435 aa, synthetic construct
MNLFLATLGSSGDVFPFIIIGEHMAKRGHKVFLCTNPYFKCLAESRGLEFISVGSAQEYIAGVHSRALWDSETALDTLCRYMLKQQDEMYQQLERVVDGSSVLLTSLWSFSAKTLGERKGCCTLPVRVGPSTFLSCYNLPQHGKLALANTLPMTLRKGIVSLFEKQVLDKRFCAEVNTLRSRVGLAPVKRILSQWVHTSADGLICLFPEWFAHPQPDWPRNANLVGLPLFEAENAPVDEELGRFLERETVLFSPGWSLQFDEYFFNAIVAEVTRQRRQCLVVGVNPEDVPVRPGVLVKKEVDLAACLRHCVAAVHHGGIGTISHCFYQSVPQLIFPSAFDQFDNAYRVEQLGCGIQAKSRLPEKFSDALDRVMTDENMRERCRRIQEKYGDKNHCLENVRHALEQAHQRHASCPGSVAYRENLYFQGDYKDDDDK

SEQ ID NO: 80 - protein, 969 aa, synthetic construct
MAGRQWSAQWIWGGEEVSPRNEWRCFRKTFYVPEGGENGARLHISADSRYVLFVNGERAGRGPVRSWPAEQFYDTYDIGRLLKPGRTNAIAVLVLHFGVANFYYIRGRGGLLAELEGGGRTGTLAATDGSWRTSRLAAQDPRSPRMSCQQGFADIYDARKWDERWTEADYDDADWPLANIVGPVGSGPWKKLVPRDIPFLTEETVYPSRVASLQAVNPPRWAAALDVRNGMLPDSANHADRVKYAGFLATVLTMRESGKLTLGFPNGRVSSVWVDGRLCGGWYGDQPERYCDLELAAGDHFVLIDVSGGDHGGSFHIAADSGSEFVLRSPAAAAAGNDTPFAIVGPFHYTIDIDHQGVEPLDLEQPDYVRAKGIKHERDLESFREWVRPLPGGMYSEDDVFGSNVWRTAAVSYAVPPELDRCVQASPEPAVVPRFDGMDTELVIDFGKEWSGFIGFEAEAPEGTVIDLYGLEYMKDDYRQHTYGLDNTIRYICRGGRQQYISPVRRGFRYLVVTIRQASAPVRLHRVFVNQSAYPVAQNGYFRSSDALLNDIWEISRHTTLLCMEDTFVDCPSYEQVFWVGDSRNEALVNYYVFGALDIVKRCLNLVPGSGEMTPLYVDQVPSAWNSVIPNWTFFWVTACAEYAAHTGDRAFAARIWPAVRHTLTHYLRHVGERGLLDIKGWNLLDWAPIDQPNDGIVTHQNMFLAKALRDAAGLASAAGVPQEAAPLAERADALRKAIDGQLWDEERQAYIDCIHADGRRSATFSMQTQVVAYLCDIAEGKRKERLEGYLVAPPASFVQIGSPFMSFFYYEALVRSGQYDRMIADIRHSYGMMIENGATTCWEMYPNFSENRANPNQLTRSHCHAWSAAPGYFLGAVVLGIRRLDDGWKRAAVAPEPCGLQWARGAVPLPQGGRIEVSWRIDGGRMELHVSAPDDVELDIRIPDGLSGSVTRSATRPSPLAAAAGMTP

SEQ ID NO: 81 - DNA, 1263 bp, Synthetic construct
  source 1..1263: mol_type = other DNA
atgcacgtgatcatcaccgccgtgggcagcgccggcgacgtgcacccgttcgtgggcatcggccgcaccctggccgcccgcggccaccgcatcaccttctgcgccagcgccgccttcgccccgctgatcgagcgctgcggcttcaagttcctgccgctgggcacccgcgaggagtacgacgaggtgatggccgacccggccctgtggcacccgcgcaccagcctgccgaccctgtggaagaccgtgggcagcaccctgcgcgagcagtaccgcctgctggagcaggcctgcgacgacgagaccgtgatgctgggcagcctgtgggccttcagcgcccgcctgctgcaggagaagcacggcatcccgctggtgaccgcccaggtgagcccgttcggcttctggagcgccgccgccccgagcacccacccgccgggcgccaacctgccggccttcgtgccgtacccggtgaagcgcctgctgctgggcctgatcgagcgcgccttcctggaccgcgtgatggccccggagctgaacagcttccgccgcgagctgggcctgccgccggtgaagcgcatcatcagccagtggatcagcagcccggaccgcgtgctgggcctgttcccggagtggttcgccccggtgcagcacgactggccggcccagaccgtgctgaccggcttcccgctgttcgacgagagcggcttccagccgccggacgccgagctggaggacttcctgaacgaggccagcccggtggtgttcaccccgggcagcaccatggtgaacagcgagcagtacttcgccgccgccgtgcaggccctgaagctgatcaaccgccgcggcgtgttcctgaccagccagccggtggacccggccgtgagcagcgagggccgcatcctggtgcgccgctacgtgccgatgagccgcatcctgccgcacagcgccgccctggtgcaccacggcggcgtgggcaccaccgccctggccatggccgccggcgtgccgcagatcgccaccccgttcgcccacgaccagttcgacaacgccgcccgcatggagcgcctgggctgcggcctgcgcgtgttcccgccggccagcgccgagagcctggccaccgccctggacaaggtgctgaacagccaggaggtgcgcgccagctgcgaccgctaccgcaccctgatcccgagcggcgacagcgccagcgaggccgccgccctgcagatcgaggccctggcccgcaccgcccgctaccgcgccgcctga

SEQ ID NO: 82 - DNA, 1263 bp, Synthetic construct
  source 1..1263: mol_type = other DNA
atgcacgtgatcatcaccgccatcggcagcgccggcgacgtgcacccgttcgtgggcatcggccgcaccctggccgcccgcggccaccgcatcaccttctgcgccagcgccgccttcgccccgctgatcgagcgctgcggcttcaagttcctgccgctgggcacccgcgaggagtacgacgaggtgatggccgacccggccctgtggcacccgcgcaccagcctgccgaccctgtggaagaccgtggccagcaccctgcgcgagcagtaccgcctgctggagcaggcctgcgacgaccagaccgtgatgctgggcagcctgtgggccttcagcgcccgcctgctgcaggagaagcgcggcatcccgctgatcaccgcccaggtgagcccgttcggcttctggagcgccgccgccccgagcacccacccgccgggcgccaacctgccggccttcgtgccgtacccggtgaagcgcctgctgatgggcgtgatcgagcgcaccttcctggaccgcgtgatggccccggagctgaaccgcttccgccgcgagctgggcctgccgccggccaagcgcatcatcagccagtggatcagcagcccggaccgcgtgctgggcctgttcccggagtggttcgccccggtgcagcgcgactggccggcccagaccgtgctgaccggcttcccgctgttcgacgagagcagcttccaggagccggacgccgagctggacgacttcctgaacgaggccagcccggtggtgttcaccccgggcagcaccatggtgaacagcgagcagtacttcgccgccgccgtgcaggccctgaagctgatcaaccgccgcggcgtgttcctgaccagccagccggtggacccgaccgtggccaccgagggccgcatcctggtgcgccgctacgtgccgatgagccgcatcctgccgcagagcgccgccctggtgcaccacggcggcgtgggcaccaccgccctggccatggccgccggcgtgccgcaggtggccaccccgttcgcccacgaccagttcgacaacgccgcccgcatggagcgcctgggctgcggcctgcgcgtgttcccgccggccagcgccgagagcctggccaccgccctggacaaggtgctgaacagccaggaggtgcgcgccagctgcgaccgctaccgcaccctgatcccgagcggcgagagcgcctgcgaggccgccgcccgccagatcgaggccatcgtggccgagccgggctaccgcgcccgctga

SEQ ID NO: 83 - protein, 489 aa, Synthetic construct
MYSFPNSFRFGWSQAGFQSEMGTPGSEDPNTDWYKWVHDPENMAAGLVSGDLPENGPGYWGNYKTFHDNAQKMGLKIARLNVEWSRIFPNPLPRPQNFDESKQDVTEVEINENELKRLDEYANKDALNHYREIFKDLKSRGLYFILNMYHWPLPLWLHDPIRVRRGDFTGPSGWLSTRTVYEFARFSAYIAWKFDDLVDEYSTMNEPNVVGGLGYVGVKSGFPPGYLSFELSRRAMYNIIQAHARAYDGIKSVSKKPVGIIYANSSFQPLTDKDMEAVEMAENDNRWWFFDAIIRGEITRGNEKIVRDDLKGRLDWIGVNYYTRTVVKRTEKGYVSLGGYGHGCERNSVSLAGLPTSDFGWEFFPEGLYDVLTKYWNRYHLYMYVTENGIADDADYQRPYYLVSHVYQVHRAINSGADVRGYLHWSLADNYEWASGFSMRFGLLKVDYNTKRLYWRPSALVYREIATNGAITDEIEHLNSVPPVKPLRH

SEQ ID NO: 84 - protein, 956 aa, Synthetic construct
MAGRNWNASWIWGGQEESPRNEWRCFRGSFDAPASVEGPAMLHITADSRYVLFVNGEQVGRGPVRSWPKEQFYDSYDIGGQLRPGVRNTIAVLVLHFGVSNFYYLRGRGGLIAEIEADGRTLAATDAAWRTERLGGQRSNSPRMACQQGFGEVIDARELAEDWALPAFDDGGWAQARSIGPAGTAPWTSLVPRDIPFLTEEKLYPASIQSLSRVKAPKYAAALDLRNQMVPESVNHANPVSYCGYVATILTLETSGVVTLGFPTGVRGSGVWVDGVLQTEWTGVQPERYYSLNLAAGEHLVLVDITSSDHGGSSHFAIDSEAAFTLRSPAGDNGVPLATIGTFDQSEYIDHRPGRRMQTDHPDYRALPEAAPTAAALEAFASWVKPFEPSLYTEENVFGSNVWRTLAERRAVPRSVLNAILPVPEPGVLPVFEDGDCELVIDLGAERSGFIGFELEAPAGTIIDAYGVEYMREGYTQHTYGLDNTFRYICREGRQSYVSPVRRGFRYLFLTVRGNSAPVKLHEIYIRQSTYPVAEQGSFRCSDALLNATWEISRHTTRLCMEDTFVDCPSYEQVFWVGDSRNEALVNYYVFGETEIVERCLNLVPGSADETPLYLDQVPSAWSSVIPNWTFFWILACREYAAHTGNEAFAARIWPAVKHTLTHYLEHIDDSGLLNMAGWNLLDWAPIDQPNEGIVTHQNLFLVKALRDSRALAAAAGATEEADAFAARADLLAETINAVLWDEEKRAYIDCIHADGRRSDVYSMQTQVVAYLCGVAQGEREAVIEGYLSSPPPAFVQIGSPFMSFFYYEALEKAGRQTLMLDDIRRNYGQMLRYDATTCWEMYPNFAENRSNPDMLTRSHCHAWSAAPGYFLGSSILGVKRGADGWRTVDIAPQPCDLTWAEGVVPLPQGGHIAVSWEFVSAGKLKLRIEAPEDIEVNVTLPEGIEGEVTQVKYMS